SEQUENCE LISTING (370 sequences)

SEQ ID NO: 1 - protein, 26 aa, synthetic construct
KRRWKKNFIAVSAANRFKKISSSGAL

SEQ ID NO: 2 - protein, 25 aa, synthetic construct
KRRWKKNFIAVSAANRFKKISSSGA

SEQ ID NO: 3 - protein, 19 aa, synthetic construct
RWKKNFIAVSAANRFKKIS

SEQ ID NO: 4 - protein, 18 aa, synthetic construct
WKKNFIAVSAANRFKKIS

SEQ ID NO: 5 - protein, 18 aa, synthetic construct
RWKKNFIAVSAANRFKKI

SEQ ID NO: 6 - protein, 17 aa, synthetic construct
RWKKNFIAVSAANRFKK

SEQ ID NO: 7 - protein, 19 aa, synthetic construct
RWKKAFIAVSAANRFKKIS

SEQ ID NO: 8 - protein, 18 aa, synthetic construct
WKKAFIAVSAANRFKKIS

SEQ ID NO: 9 - protein, 18 aa, synthetic construct
RWKKAFIAVSAANRFKKI

SEQ ID NO: 10 - protein, 17 aa, synthetic construct
RWKKAFIAVSAANRFKK

SEQ ID NO: 11 - protein, 19 aa, synthetic construct
RWKKNIIAVSAANRFKKIS

SEQ ID NO: 12 - protein, 19 aa, synthetic construct
RWKKNFIKVSAANRFKKIS

SEQ ID NO: 13 - protein, 19 aa, synthetic construct
RWKKNFIAVLAANRFKKIS

SEQ ID NO: 14 - protein, 19 aa, synthetic construct
RWKKNFIAVSAINRFKKIS

SEQ ID NO: 15 - protein, 19 aa, synthetic construct
RWKKAIIKVLAINRFKKIS

SEQ ID NO: 16 - protein, 19 aa, synthetic construct
RWKKAIIKVLAANRFKKIS

SEQ ID NO: 17 - protein, 19 aa, synthetic construct
RWKKAIIKVSAANRFKKIS

SEQ ID NO: 18 - protein, 19 aa, synthetic construct
RWKKAIIAVSAANRFKKIS

SEQ ID NO: 19 - protein, 19 aa, synthetic construct
RWKKAFIKVLAINRFKKIS

SEQ ID NO: 20 - protein, 19 aa, synthetic construct
RWKKAFIAVLAINRFKKIS

SEQ ID NO: 21 - protein, 19 aa, synthetic construct
RWKKAFIAVSAINRFKKIS

SEQ ID NO: 22 - protein, 19 aa, synthetic construct
RWKKNIIKVLAINRFKKIS

SEQ ID NO: 23 - protein, 19 aa, synthetic construct
RWKKNFIKVLAINRFKKIS

SEQ ID NO: 24 - protein, 19 aa, synthetic construct
RWKKNFIAVLAINRFKKIS

SEQ ID NO: 25 - protein, 19 aa, synthetic construct
RWKKAIIKVSAINRFKKIS

SEQ ID NO: 26 - protein, 19 aa, synthetic construct
RWKKAIIAVSAINRFKKIS

SEQ ID NO: 27 - protein, 19 aa, synthetic construct
RWKKAIIAVLAINRFKKIS

SEQ ID NO: 28 - protein, 19 aa, synthetic construct
RWKKAFIKVSAANRFKKIS

SEQ ID NO: 29 - protein, 19 aa, synthetic construct
RWKKAFIAVLAANRFKKIS

SEQ ID NO: 30 - protein, 19 aa, synthetic construct
RWKKAIIAVLAANRFKKIS

SEQ ID NO: 31 - protein, 19 aa, synthetic construct
RWKKAFIKVLAANRFKKIS

SEQ ID NO: 32 - protein, 19 aa, synthetic construct
RWKKAFIKVSAINRFKKIS

SEQ ID NO: 33 - protein, 19 aa, synthetic construct
RWKKNIIKVLAANRFKKIS

SEQ ID NO: 34 - protein, 19 aa, synthetic construct
RWKKNIIKVSAANRFKKIS

SEQ ID NO: 35 - protein, 19 aa, synthetic construct
RWKKNIIAVLAINRFKKIS

SEQ ID NO: 36 - protein, 19 aa, synthetic construct
RWKKNIIAVLAANRFKKIS

SEQ ID NO: 37 - protein, 19 aa, synthetic construct
RWKKNIIAVSAINRFKKIS

SEQ ID NO: 38 - protein, 19 aa, synthetic construct
RWKKNDIAVSAANRFKKIS

SEQ ID NO: 39 - protein, 19 aa, synthetic construct
RWKKNFIDVSAANRFKKIS

SEQ ID NO: 40 - protein, 19 aa, synthetic construct
RWKKNFIAVHAANRFKKIS

SEQ ID NO: 41 - protein, 19 aa, synthetic construct
RWKKNFIAVSARNRFKKIS

SEQ ID NO: 42 - protein, 17 aa, synthetic construct
GVRNIKSMWEKGNVFSS

SEQ ID NO: 43 - protein, 20 aa, synthetic construct
ARRKLKAAVKAVVASSRLGS

SEQ ID NO: 44 - protein, 25 aa, synthetic construct
FMNNWEVYKLLAHIRPPAPKSGSYT

SEQ ID NO: 45 - protein, 24 aa, synthetic construct
ARKEVIRNKIRAIGKMARVFSVLR

SEQ ID NO: 46 - protein, 24 aa, synthetic construct
RGQILWFRGLNRIQTQIKVVNAFS

SEQ ID NO: 47 - protein, 24 aa, synthetic construct
RRKHLQRPIFRLRCLVKQLEKPDE

SEQ ID NO: 48 - protein, 24 aa, synthetic construct
KTASPWKSARLMVHTVATFNSIKE

SEQ ID NO: 49 - protein, 25 aa, synthetic construct
KKKKKRFSFKKSFKLSGFSFKKSKK

SEQ ID NO: 50 - protein, 24 aa, synthetic construct
KKKATFRAITSTLASSFKRRRSSK

SEQ ID NO: 51 - protein, 23 aa, synthetic construct
KRRAIGFKKLAEAVKFSAKLMGQ

SEQ ID NO: 52 - protein, 20 aa, synthetic construct
RKKTFKEVANAVKISASLMG

SEQ ID NO: 53 - protein, 25 aa, synthetic construct
AAARKEVIRNKIRAIGKMARVFSVL

SEQ ID NO: 54 - protein, 19 aa, synthetic construct
KIQASFRGHMARKKIKSGE

SEQ ID NO: 55 - protein, 25 aa, synthetic construct
KAATKIQASFRGHITRKKLKGEKKG

SEQ ID NO: 56 - protein, 20 aa, synthetic construct
LRRGQILWFRGLNRIQTQIK

SEQ ID NO: 57 - protein, 25 aa, synthetic construct
SKYITTIAGVMTLSQVKGFVRKNGV

SEQ ID NO: 58 - protein, 17 aa, synthetic construct
ATLDALLAALRRIQRAD

SEQ ID NO: 59 - protein, 19 aa, synthetic construct
RAANLWPSPLMIKRSKKNS

SEQ ID NO: 60 - protein, 21 aa, synthetic construct
KIYAAMMIMEYYRQSKAKKLQ

SEQ ID NO: 61 - protein, 21 aa, synthetic construct
KIYAAMMIMDYYKQSKVKKQR

SEQ ID NO: 62 - protein, 21 aa, synthetic construct
KFYATFLIQEHFRKFMKRQEE

SEQ ID NO: 63 - protein, 24 aa, synthetic construct
GTGAALSWQAAIDAARQAKLMGSA

SEQ ID NO: 64 - protein, 23 aa, synthetic construct
HMGKVYAALMIFDFYKQNKTSRD

SEQ ID NO: 65 - protein, 23 aa, synthetic construct
GHMGKIYAAMMIMDYYKQSKVKK

SEQ ID NO: 66 - protein, 22 aa, synthetic construct
HMGKIYAAMMIMEYYRQSKAKK

SEQ ID NO: 67 - protein, 25 aa, synthetic construct
IKKNFAKSKWKQAFNATAVVRHMRK

SEQ ID NO: 68 - protein, 25 aa, synthetic construct
LKKFNARRKLKGAILTTMLATRNFS

SEQ ID NO: 69 - protein, 20 aa, synthetic construct
HSMQALSWRKLYLSRAKLKA

SEQ ID NO: 70 - protein, 22 aa, synthetic construct
AKSKWKQAFNATAVVRHMRKLQ

SEQ ID NO: 71 - protein, 20 aa, synthetic construct
ARRKWQKTGHAVRAIGRLSS

SEQ ID NO: 72 - protein, 20 aa, synthetic construct
ARRKLKGAILTTMLATRNFS

SEQ ID NO: 73 - protein, 17 aa, synthetic construct
GVRNIKSMWEKGNVFSS

SEQ ID NO: 74 - protein, 20 aa, synthetic construct
ARRKLKAAVKAVVASSRLGS

SEQ ID NO: 75 - protein, 26 aa, synthetic construct
FMNNWEVYKLLAHIRPPAPKSGSYTV

SEQ ID NO: 76 - protein, 24 aa, synthetic construct
ARKEVIRNKIRAIGKMARVFSVLR

SEQ ID NO: 77 - protein, 24 aa, synthetic construct
RGQILWFRGLNRIQTQIKVVNAFS

SEQ ID NO: 78 - protein, 21 aa, synthetic construct
RRKHLQRPIFRLRCLVKQLEK

SEQ ID NO: 79 - protein, 23 aa, synthetic construct
KRRAIGFKKLAEAVKFSAKLMGQ

SEQ ID NO: 80 - protein, 27 aa, synthetic construct
KAHKAATKIQASFRGHITRKKLKGEKK

SEQ ID NO: 81 - protein, 24 aa, synthetic construct
KTASPWKSARLMVHTVATFNSIKE

SEQ ID NO: 82 - protein, 25 aa, synthetic construct
KKKKKRFSFKKSFKLSGFSFKKSKK

SEQ ID NO: 83 - protein, 14 aa, synthetic construct
INLKALAALAKKIL

SEQ ID NO: 84 - protein, 25 aa, synthetic construct
GIGGAVLKVLTTGLPALISWIKRKR

SEQ ID NO: 85 - protein, 14 aa, synthetic construct
VDWKKIGQHILSVL

SEQ ID NO: 86 - protein, 123 aa, Mus musculus
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVS

SEQ ID NO: 87 - protein, 124 aa, Mus musculus
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQL

SEQ ID NO: 88 - protein, 125 aa, Mus musculus
EVQLVESGGGLVQPGGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 89 - protein, 109 aa, Mus musculus
QAVVTQESALTTSPGETVTLTCRSSTGAVTTSNYANWVQEKPDHLFTGLIGGTNKRAPGVPARFSGSLIGGKAALTITGAQTEDEAIYFCALWYSNLWVFGGGTKLTVL

SEQ ID NO: 90 - protein, 122 aa, Mus musculus
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 91 - protein, 107 aa, Mus musculus
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 92 - protein, 148 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 93 - protein, 148 aa, Homo sapiens; identical to SEQ ID NO: 92

SEQ ID NO: 94 - protein, 147 aa, Homo sapiens
DQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 95 - protein, 152 aa, Homo sapiens
MKDTDSEEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARK

SEQ ID NO: 96 - protein, 152 aa, Homo sapiens
SEEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTD

SEQ ID NO: 97 - protein, 152 aa, Homo sapiens
KGGSGADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTA

SEQ ID NO: 98 - protein, 152 aa, Homo sapiens
AKGGSGADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMT

SEQ ID NO: 99 - protein, 145 aa, Homo sapiens
LTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 100 - protein, 142 aa, Homo sapiens
EQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 101 - protein, 139 aa, Homo sapiens
AEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 102 - protein, 134 aa, Homo sapiens
AFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 103 - protein, 129 aa, Homo sapiens
DKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 104 - protein, 124 aa, Homo sapiens
GTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 105 - protein, 119 aa, Homo sapiens
KELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 106 - protein, 109 aa, Homo sapiens
GQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 107 - protein, 143 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQ

SEQ ID NO: 108 - protein, 140 aa, Homo sapiens
LTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQ

SEQ ID NO: 109 - protein, 137 aa, Homo sapiens
EQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQ

SEQ ID NO: 110 - protein, 134 aa, Homo sapiens
AEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQ

SEQ ID NO: 111 - protein, 129 aa, Homo sapiens
AFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQ

SEQ ID NO: 112 - protein, 124 aa, Homo sapiens
DKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQ

SEQ ID NO: 113 - protein, 119 aa, Homo sapiens
GTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQ

SEQ ID NO: 114 - protein, 114 aa, Homo sapiens
KELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQ

SEQ ID NO: 115 - protein, 104 aa, Homo sapiens
GQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQ

SEQ ID NO: 116 - protein, 143 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVK

SEQ ID NO: 117 - protein, 140 aa, Homo sapiens
LTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVK

SEQ ID NO: 118 - protein, 137 aa, Homo sapiens
EQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVK

SEQ ID NO: 119 - protein, 134 aa, Homo sapiens
AEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVK

SEQ ID NO: 120 - protein, 129 aa, Homo sapiens
AFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVK

SEQ ID NO: 121 - protein, 124 aa, Homo sapiens
DKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVK

SEQ ID NO: 122 - protein, 119 aa, Homo sapiens
GTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVK

SEQ ID NO: 123 - protein, 114 aa, Homo sapiens
KELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVK

SEQ ID NO: 124 - protein, 104 aa, Homo sapiens
GQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVK

SEQ ID NO: 125 - protein, 138 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNY

SEQ ID NO: 126 - protein, 135 aa, Homo sapiens
LTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNY

SEQ ID NO: 127 - protein, 132 aa, Homo sapiens
EQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNY

SEQ ID NO: 128 - protein, 129 aa, Homo sapiens
AEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNY

SEQ ID NO: 129 - protein, 124 aa, Homo sapiens
AFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNY

SEQ ID NO: 130 - protein, 119 aa, Homo sapiens
DKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNY

SEQ ID NO: 131 - protein, 114 aa, Homo sapiens
GTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNY

SEQ ID NO: 132 - protein, 109 aa, Homo sapiens
KELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNY

SEQ ID NO: 133 - protein, 99 aa, Homo sapiens
GQNPTEAELQDMINEVDADGNGTIDFPEFLTMMARKMKDTDSEEEIREAFRVFDKDGNGYISAAELRHVMTNLGEKLTDEEVDEMIREADIDGDGQVNY

SEQ ID NO: 134 - protein, 152 aa, Homo sapiens; identical to SEQ ID NO: 96

SEQ ID NO: 135 - protein, 149 aa, Homo sapiens
EIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTD

SEQ ID NO: 136 - protein, 142 aa, Homo sapiens
VFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTD

SEQ ID NO: 137 - protein, 137 aa, Homo sapiens
GNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTD

SEQ ID NO: 138 - protein, 132 aa, Homo sapiens
SAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTD

SEQ ID NO: 139 - protein, 127 aa, Homo sapiens
RHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTD

SEQ ID NO: 140 - protein, 122 aa, Homo sapiens
NLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTD

SEQ ID NO: 141 - protein, 112 aa, Homo sapiens
VDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTD

SEQ ID NO: 142 - protein, 147 aa, Homo sapiens
SEEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARK

SEQ ID NO: 143 - protein, 144 aa, Homo sapiens
EIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARK

SEQ ID NO: 144 - protein, 137 aa, Homo sapiens
VFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARK

SEQ ID NO: 145 - protein, 132 aa, Homo sapiens
GNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARK

SEQ ID NO: 146 - protein, 127 aa, Homo sapiens
SAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARK

SEQ ID NO: 147 - protein, 122 aa, Homo sapiens
RHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARK

SEQ ID NO: 148 - protein, 117 aa, Homo sapiens
NLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARK

SEQ ID NO: 149 - protein, 107 aa, Homo sapiens
VDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARK

SEQ ID NO: 150 - protein, 147 aa, Homo sapiens
SEEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARD

SEQ ID NO: 151 - protein, 142 aa, Homo sapiens
SEEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLT

SEQ ID NO: 152 - protein, 139 aa, Homo sapiens
EIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLT

SEQ ID NO: 153 - protein, 132 aa, Homo sapiens
VFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLT

SEQ ID NO: 154 - protein, 127 aa, Homo sapiens
GNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLT

SEQ ID NO: 155 - protein, 122 aa, Homo sapiens
SAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLT

SEQ ID NO: 156 - protein, 117 aa, Homo sapiens
RHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLT

SEQ ID NO: 157 - protein, 112 aa, Homo sapiens
NLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLT

SEQ ID NO: 158 - protein, 102 aa, Homo sapiens
VDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLT

SEQ ID NO: 159 - protein, 152 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKGGSG

SEQ ID NO: 160 - protein, 149 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKG

SEQ ID NO: 161 - protein, 145 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMM

SEQ ID NO: 162 - protein, 147 aa, Homo sapiens
EEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKGGSG

SEQ ID NO: 163 - protein, 98 aa, Homo sapiens
DKDGDGTITTKELGTVMRSLGQNPTEAELQDMINEVDADGNGTIDFPEFLTMMARKMKDTDSEEEIREAFRVFDKDGNGYISAAELRHVMTNLGEKLT

SEQ ID NO: 164 - protein, 89 aa, Homo sapiens
GQNPTEAELQDMINEVDADGNGTIDFPEFLTMMARKMKDTDSEEEIREAFRVFDKDGNGYISAAELRHVMTNLGEKLTDEEVDEMIREA

SEQ ID NO: 165 - protein, 113 aa, Homo sapiens
GQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKGGSG

SEQ ID NO: 166 - protein, 69 aa, Homo sapiens
NGTIDFPEFLTMMARKMKDTDSEEEIREAFRVFDKDGNGYISAAELRHVMTNLGEKLTDEEVDEMIREA

SEQ ID NO: 167 - protein, 93 aa, Homo sapiens
NGTIDFPEFLTMMARKMKDTDSEEEIREAFRVFDKDGNGYISAAELRHVMTNLGEKLTDEEVDEMIREADIDGDGQVNYEEFVQMMTAKGGSG

SEQ ID NO: 168 - protein, 36 aa, Homo sapiens
DKDGDGYISAAELRHVMTNLGEKLTDEEVDEMIREA

SEQ ID NO: 169 - protein, 20 aa, Homo sapiens
MTNLGEKLTDEEVDEMIREA

SEQ ID NO: 170 - protein, 40 aa, Homo sapiens
MTNLGEKLTDEEVDEMIREADIDGDGQVNYEEFVQMMTAK

SEQ ID NO: 171 - protein, 107 aa, Homo sapiens
DGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREA

SEQ ID NO: 172 - protein, 58 aa, Homo sapiens
NGTIDFPEFLTMMARKMKDTDSEEEIREAFRVFDKDGNGYISAAELRHVMTNLGEKLT

SEQ ID NO: 173 - protein, 8 aa, Homo sapiens
DKDGEKLT

SEQ ID NO: 174 - protein, 20 aa, Homo sapiens
DKDGDGQVNYEEFVQMMTAK

SEQ ID NO: 175 - protein, 152 aa, Homo sapiens; identical to SEQ ID NO: 97

SEQ ID NO: 176 - protein, 152 aa, Homo sapiens; identical to SEQ ID NO: 98

SEQ ID NO: 177 - protein, 80 aa, Homo sapiens
ADQLTEEQIAEFKEAFSLFDKDGDGTITTKELGTVMRSLGQNPTEAELQDMINEVDADGNGTIDFPEFLTMMARKMKDTD

SEQ ID NO: 178 - protein, 70 aa, Homo sapiens
EFKEAFSLFDKDGDGTITTKELGTVMRSLGQNPTEAELQDMINEVDADGNGTIDFPEFLTMMARKMKDTD

SEQ ID NO: 179 - protein, 21 aa, Homo sapiens
NGTIDFPEFLTMMARKMKDTD

SEQ ID NO: 180 - protein, 124 aa, Homo sapiens
MTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTD

SEQ ID NO: 181 - protein, 149 aa, Homo sapiens
SEEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMK

SEQ ID NO: 182 - protein, 149 aa, Homo sapiens; identical to SEQ ID NO: 135

SEQ ID NO: 183 - protein, 150 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSG

SEQ ID NO: 184 - protein, 149 aa, Homo sapiens
DQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSG

SEQ ID NO: 185 - protein, 148 aa, Homo sapiens
QLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSG

SEQ ID NO: 186 - protein, 147 aa, Homo sapiens
LTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSG

SEQ ID NO: 187 - protein, 146 aa, Homo sapiens
TEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSG

SEQ ID NO: 188 - protein, 145 aa, Homo sapiens
EEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSG

SEQ ID NO: 189 - protein, 144 aa, Homo sapiens
EQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSG

SEQ ID NO: 190 - protein, 143 aa, Homo sapiens
QIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSG

SEQ ID NO: 191 - protein, 141 aa, Homo sapiens
AEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKSG

SEQ ID NO: 192 - protein, 149 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTASG

SEQ ID NO: 193 - protein, 148 aa, Homo sapiens
DQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTASG

SEQ ID NO: 194 - protein, 147 aa, Homo sapiens
QLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTASG

SEQ ID NO: 195 - protein, 146 aa, Homo sapiens
LTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTASG

SEQ ID NO: 196 - protein, 145 aa, Homo sapiens
TEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTASG

SEQ ID NO: 197 - protein, 144 aa, Homo sapiens
EEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTASG

SEQ ID NO: 198 - protein, 143 aa, Homo sapiens
EQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTASG

SEQ ID NO: 199 - protein, 142 aa, Homo sapiens
QIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTASG

SEQ ID NO: 200 - protein, 140 aa, Homo sapiens
AEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTASG

SEQ ID NO: 201 - protein, 148 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTSG

SEQ ID NO: 202 - protein, 147 aa, Homo sapiens
DQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTSG

SEQ ID NO: 203 - protein, 146 aa, Homo sapiens
QLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTSG

SEQ ID NO: 204 - protein, 145 aa, Homo sapiens
LTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTSG

SEQ ID NO: 205 - protein, 144 aa, Homo sapiens
TEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTSG

SEQ ID NO: 206 - protein, 143 aa, Homo sapiens
EEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTSG

SEQ ID NO: 207 - protein, 142 aa, Homo sapiens
EQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTSG

SEQ ID NO: 208 - protein, 141 aa, Homo sapiens
QIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTSG

SEQ ID NO: 209 - protein, 139 aa, Homo sapiens
AEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTSG

SEQ ID NO: 210 - protein, 147 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMSG

SEQ ID NO: 211 - protein, 146 aa, Homo sapiens
DQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMSG

SEQ ID NO: 212 - protein, 145 aa, Homo sapiens
QLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMSG

SEQ ID NO: 213 - protein, 144 aa, Homo sapiens
LTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMSG

SEQ ID NO: 214 - protein, 143 aa, Homo sapiens
TEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMSG

SEQ ID NO: 215 - protein, 142 aa, Homo sapiens
EEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMSG

SEQ ID NO: 216 - protein, 141 aa, Homo sapiens
EQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMSG

SEQ ID NO: 217 - protein, 140 aa, Homo sapiens
QIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMSG

SEQ ID NO: 218 - protein, 138 aa, Homo sapiens
AEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMSG

SEQ ID NO: 219 - protein, 146 aa, Homo sapiens
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMSG

SEQ ID NO: 220 - protein, 145 aa, Homo sapiens
DQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMSG

SEQ ID NO: 221 - protein, 144 aa, Homo sapiens
QLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMSG

SEQ ID NO: 222 - protein, 143 aa, Homo sapiens
LTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMSG

SEQ ID NO: 223 - protein, 142 aa, Homo sapiens
TEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMSG

SEQ ID NO: 224 - protein, 141 aa, Homo sapiens
EEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMSG

SEQ ID NO: 225 - protein, 140 aa, Homo sapiens
EQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMSG

SEQ ID NO: 226 - protein, 139 aa, Homo sapiens
QIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMSG

SEQ ID NO: 227 - protein, 137 aa, Homo sapiens
AEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMSG

SEQ ID NO: 228 - protein, 154 aa, Homo sapiens
SEEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTDSG

SEQ ID NO: 229 - protein, 153 aa, Homo sapiens
EEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTDSG

SEQ ID NO: 230 - protein, 152 aa, Homo sapiens
EEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTDSG

SEQ ID NO: 231 - protein, 151 aa, Homo sapiens
EIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTDSG

SEQ ID NO: 232 - protein, 150 aa, Homo sapiens
IREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTDSG

SEQ ID NO: 233 - protein, 149 aa, Homo sapiens
REAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTDSG

SEQ ID NO: 234 - protein, 148 aa, Homo sapiens
EAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTDSG

SEQ ID NO: 235 - protein, 147 aa, Homo sapiens
AFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTDSG

SEQ ID NO: 236 - protein, 144 aa, Homo sapiens
VFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTDSG

SEQ ID NO: 237 - protein, 139 aa, Homo sapiens
GNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTDSG

SEQ ID NO: 238 - protein, 153 aa, Homo sapiens
SEEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTSG

SEQ ID NO: 239 - protein, 152 aa, Homo sapiens
EEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTSG

SEQ ID NO: 240 - protein, 151 aa, Homo sapiens
EEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTSG

SEQ ID NO: 241 - protein, 150 aa, Homo sapiens
EIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTSG

SEQ ID NO: 242 - protein, 149 aa, Homo sapiens
IREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTSG

SEQ ID NO: 243 - protein, 148 aa, Homo sapiens
REAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTSG

SEQ ID NO: 244 - protein, 147 aa, Homo sapiens
EAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTSG

SEQ ID NO: 245 - protein, 146 aa, Homo sapiens
AFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTSG

SEQ ID NO: 246 - protein, 143 aa, Homo sapiens
VFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTSG

SEQ ID NO: 247 - protein, 138 aa, Homo sapiens
GNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTSG

SEQ ID NO: 248 - protein, 152 aa, Homo sapiens
SEEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDSG

SEQ ID NO: 249 - protein, 151 aa, Homo sapiens
EEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDSG

SEQ ID NO: 250 - protein, 150 aa, Homo sapiens
EEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDSG

SEQ ID NO: 251 - protein, 149 aa, Homo sapiens
EIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDSG

SEQ ID NO: 252 - protein, 148 aa, Homo sapiens
IREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDSG

SEQ ID NO: 253 - protein, 147 aa, Homo sapiens
REAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDSG

SEQ ID NO: 254 - protein, 146 aa, Homo sapiens
EAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDSG

SEQ ID NO: 255 - protein, 145 aa, Homo sapiens
AFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDSG

SEQ ID NO: 256 - protein, 142 aa, Homo sapiens
VFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDTG

SEQ ID NO: 257 - protein, 137 aa, Homo sapiens
GNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKDSG

SEQ ID NO: 258 - protein, 151 aa, Homo sapiens
SEEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKSG

SEQ ID NO: 259 - protein, 150 aa, Homo sapiens
EEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKSG

SEQ ID NO: 260 - protein, 149 aa, Homo sapiens
EEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKSG

SEQ ID NO: 261 - protein, 148 aa, Homo sapiens
EIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKSG

SEQ ID NO: 262 - protein, 147 aa, Homo sapiens
IREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKSG

SEQ ID NO: 263 - protein, 146 aa, Homo sapiens
REAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKSG

SEQ ID NO: 264 - protein, 145 aa, Homo sapiens
EAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKSG

SEQ ID NO: 265 - protein, 144 aa, Homo sapiens
AFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKSG

SEQ ID NO: 266 - protein, 141 aa, Homo sapiens
VFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKTG

SEQ ID NO: 267 - protein, 136 aa, Homo sapiens
GNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMKSG

SEQ ID NO: 268 - protein, 150 aa, Homo sapiens
SEEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMSG

SEQ ID NO: 269 - protein, 149 aa, Homo sapiens
EEEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMSG

SEQ ID NO: 270 - protein, 148 aa, Homo sapiens
EEIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMSG

SEQ ID NO: 271 - protein, 147 aa, Homo sapiens
EIREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMSG

SEQ ID NO: 272 - protein, 146 aa, Homo sapiens
IREAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMSG

SEQ ID NO: 273 - protein, 145 aa, Homo sapiens
REAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMSG

SEQ ID NO: 274 - protein, 144 aa, Homo sapiens
EAFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMSG

SEQ ID NO: 275 - protein, 143 aa, Homo sapiens
AFRVFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMSG

SEQ ID NO: 276 - protein, 140 aa, Homo sapiens
VFDKDGNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMTG

SEQ ID NO: 277 - protein, 135 aa, Homo sapiens
GNGYISAAELRHVMTNLGEKLTDEEVDEMIREADIDGDGQVNYEEFVQMMTAKGGSGADQLTEEQIAEFKEAFSLFDKDGDGTITTKELGTVMRSLGQNPTEAELQDMINEVDADGNGTIDFPEFLTMMARKMSG

SEQ ID NO: 278 - protein, 152 aa, Homo sapiens
SDQLTEEQIAEFKEAFSLFDKDGDGTITTKELGTVMRSLGQNPTEAELQDMINEVDADGNDTIDFPEFLTMMARKMKDTDSEEEIREAFRVFDKDGNGYISAAELRHVMTNLGEKLTDEEVDEMIREADNDGDGQVNYEEFVQMMTAKGGSG

SEQ ID NO: 279 - protein, 119 aa, Homo sapiens
GTITTKELGTVMRSLGQNPTEAELQDMINEVDSDGNGTIDFPEFLTMMARKMKDTDSEEEIREAFRVFDKDGNGYISAAELRHVMTNLGEKLTDEEVDEMIREADIDGDGQVNYEEFVK

SEQ ID NO: 280 - protein, 152 aa, Homo sapiens
ADQLTEEQVAEFKEAFSLFDKDGDGTITTKELGTVMRSLGQNPTEAELQDMINEVDADGNGTIDFPEFLTMMARKMMDTDSEEEIREAFRVFDKDGNGYISAAELRHVMTNLGEKLTDEEVDEMIREADIDGDGQVNYEEFVQMMTAKGGSG

SEQ ID NO: 281 - protein, 119 aa, Homo sapiens
GTITTKELGTVMRSLGQNPTEAELQDMINEVDADGNGTIDFPEFLTMMARKMKDTDSGEEIREAFRVFDKDGNGYISAAELRHVMTNLGEKLTDEEVDEMIREADIDGDGQVNYEEFVK

SEQ ID NO: 282 - protein, 119 aa, Homo sapiens
GTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDDDGQVNYEEFVK

SEQ ID NO: 283 - protein, 119 aa, Homo sapiens
GTITTKELGTVMRSLGQNPTEAELQDMINEVDADGNGTIDFPEFLTMMARKMKDTDSEEEIREAFRVFDKDGNGYISAAELRHVMTNLSEKLTDEEVDEMIREADIDGDGQVNYEEFVK

SEQ ID NO: 284 - protein, 119 aa, Homo sapiens
GTITTKELGTVMRSLGQNPTEAELQDMIIEVDADGNGTIDFPEFLTMMARKMKDTDSEEEIREAFRVFDKDGNGYISAAELRHVMTNLGEKLTDEEVDEMIREADIDGDGQVNYEEFVK

SEQ ID NO: 285 - protein, 119 aa, Homo sapiens
GMITTKELGTVMRSLGQNPTEAELQDMINEVDADGNGTIDFPEFLTMMTRKMKDTDSEEEIREVFRVFDKDGNGYISAAELRHVMTNLGEKLTDEEVDEMIREADIGGDGQVNYEEFVK

SEQ ID NO: 286 - protein, 119 aa, Homo sapiens
GTITTKELGTVMRSLGQNPTEVELQDMINEVDADGNGTIDFPEFLTMMARKMKDTDSEEEIREAFRVFDKDGNGYISAAELRHVMTNLGEKLTDEEVDEMIREADIDGEGQVNYEEFVK

SEQ ID NO: 287 - protein, 119 aa, Homo sapiens
GTITTKELGTVMRSLGQNPTEAELQDMINEVDADGNDTIDFPEFLTMMARKMKDTDSEEEIREAFRVFDKDGNGYISAAELRHVMTNLGEKLTDEEVDEMIREADIDGDGQVNYEEFVK

SEQ ID NO: 288 - protein, 123 aa, Homo sapiens; identical to SEQ ID NO: 86

SEQ ID NO: 289 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 290 - protein, 124 aa, Homo sapiens
EVQLVESGGGLVQPGGSLKLSCAASGFTFNKYAINWVRQAPGKGLEWVARIRSKYNNYATYYADQVKDRFTISRDDSKNTAYLQMNNLTEDTAVYYCVRHANFGNSYISYWAYWGQGTLVTVSS

SEQ ID NO: 291 - protein, 109 aa, Homo sapiens
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 292 - protein, 103 aa, Homo sapiens
EVQLVQSGAEVKKPGAYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 293 - protein, 112 aa, Homo sapiens
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 294 - protein, 121 aa, Homo sapiens
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS

SEQ ID NO: 295 - protein, 106 aa, Homo sapiens
DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQIT

SEQ ID NO: 296 - protein, 119 aa, Homo sapiens
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 297 - protein, 119 aa, Homo sapiens
DVQLVQSGAEVKKPGASVKVSCKASGYTFTRYTMHWVRQAPGQGLEWIGYINPSRGYTNYNQKFKDRVTITTDKSTSTAYMELSSLRSEDTAVYYCARYYDDHYCLDYWGQGTTVTVSS

SEQ ID NO: 298 - protein, 106 aa, Homo sapiens
DIQMTQSPSSLSASVGDRVTITCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 299 - protein, 106 aa, Homo sapiens
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 300 - protein, 106 aa, Homo sapiens
DIVLTQSPATLSLSPGERATLTCRASSSVSYMNWYQQKPGKAPKRWIYDTSKVASGVPARFSGSGSGTDYSLTINSLEAEDAATYYCQQWSSNPLTFGGGTKVEIK

SEQ ID NO: 301 - protein, 5 aa, Homo sapiens
RYTMH

SEQ ID NO: 302 - protein, 17 aa, Homo sapiens
YINPSRGYTNYNQKFKD

SEQ ID NO: 303 - protein, 10 aa, Homo sapiens
YYDDHYCLDY

SEQ ID NO: 304 - protein, 10 aa, Homo sapiens
SASSSVSYMN

SEQ ID NO: 305 - protein, 7 aa, Homo sapiens
DTSKLAS

SEQ ID NO: 306 - protein, 9 aa, Homo sapiens
QQWSSNPFT

SEQ ID NO: 307 - protein, 5 aa, Homo sapiens
TYAMN

SEQ ID NO: 308 - protein, 19 aa, Homo sapiens
RIRSKYNNYATYYADSVKD

SEQ ID NO: 309 - protein, 14 aa, Homo sapiens
HGNFGNSYVSWFAY

SEQ ID NO: 310 - protein, 14 aa, Homo sapiens
RSSTGAVTTSNYAN

SEQ ID NO: 311 - protein, 7 aa, Homo sapiens
GTNKRAP

SEQ ID NO: 312 - protein, 9 aa, Homo sapiens
ALWYSNLWV

SEQ ID NO: 313 - protein, 5 aa, Homo sapiens
GYTMN

SEQ ID NO: 314 - protein, 17 aa, Homo sapiens
LINPYKGVSTYNQKFKD

SEQ ID NO: 315 - protein, 13 aa, Homo sapiens
SGYYGDSDWYFDV

SEQ ID NO: 316 - protein, 11 aa, Homo sapiens
RASQDIRNYLN

SEQ ID NO: 317 - protein, 7 aa, Homo sapiens
YTSRLHS

SEQ ID NO: 318 - protein, 9 aa, Homo sapiens
QQGNTLPWT

SEQ ID NO: 319 - protein, 5 aa, Homo sapiens
DYSMH

SEQ ID NO: 320 - protein, 17 aa, Homo sapiens
GISWNSGSIGYADSVKG

SEQ ID NO: 321 - protein, 15 aa, Homo sapiens
YGSGYGKFYYYGMDV

SEQ ID NO: 322 - protein, 11 aa, Homo sapiens
RASQSISSYLN

SEQ ID NO: 323 - protein, 7 aa, Homo sapiens
AASSLQS

SEQ ID NO: 324 - protein, 10 aa, Homo sapiens
QQSYSTPPIT

SEQ ID NO: 325 - protein, 5 aa, Homo sapiens
KYAIN

SEQ ID NO: 326 - protein, 19 aa, Homo sapiens
RIRSKYNNYATYYADQVKD

SEQ ID NO: 327 - protein, 14 aa, Homo sapiens
HANFGNSYISYWAY

SEQ ID NO: 328 - protein, 14 aa, Homo sapiens
ASSTGAVTSGNYPN

SEQ ID NO: 329 - protein, 7 aa, Homo sapiens
GTKFLVP

SEQ ID NO: 330 - protein, 10 aa, Homo sapiens
TLWYSNRWVT

SEQ ID NO: 331 - protein, 11 aa, Homo sapiens
AEVKKPGAYIH

SEQ ID NO: 332 - protein, 17 aa, Homo sapiens
WIYPGDGNTKYNEKFKG

SEQ ID NO: 333 - protein, 10 aa, Homo sapiens
DSYSNYYFDY

SEQ ID NO: 334 - protein, 17 aa, Homo sapiens
KSSQSLLNSRTRKNYLA

SEQ ID NO: 335 - protein, 7 aa, Homo sapiens
WASTRES

SEQ ID NO: 336 - protein, 8 aa, Homo sapiens
TQSFILRT

SEQ ID NO: 337 - protein, 5 aa, Homo sapiens
RSTMH

SEQ ID NO: 338 - protein, 17 aa, Homo sapiens
YINPSSAYTNYNQKFKD

SEQ ID NO: 339 - protein, 12 aa, Homo sapiens
PQVHYDYNGFPY

SEQ ID NO: 340 - protein, 10 aa, Homo sapiens
SASSSVSYMN

SEQ ID NO: 341 - protein, 7 aa, Homo sapiens
DSSKLAS

SEQ ID NO: 342 - protein, 9 aa, Homo sapiens
QQWSRNPPT

SEQ ID NO: 343 - protein, 5 aa, Homo sapiens
RYTMH

SEQ ID NO: 344 - protein, 17 aa, Homo sapiens
YINPSRGYTNYADSVKG

SEQ ID NO: 345 - protein, 10 aa, Homo sapiens
YYDDHYCLDY

SEQ ID NO: 346 - protein, 5 aa, Homo sapiens
RYTMH

SEQ ID NO: 347 - protein, 10 aa, Homo sapiens
RASQSVSYMN

SEQ ID NO: 348 - protein, 10 aa, Homo sapiens
RASSSVSYMN

SEQ ID NO: 349 - protein, 7 aa, Homo sapiens
DTSKVAS

SEQ ID NO: 350 - protein, 10 aa, Homo sapiens
QQWSSNPLTT

SEQ ID NO: 351 - protein, 111 aa, Homo sapiens [sequence omitted]

SEQ ID NO: 352 - protein, 116 aa, Homo sapiens [sequence omitted]

SEQ ID NO: 353 - protein, 7 aa, Homo sapiens
GGSISSY

SEQ ID NO: 354 - protein, 5 aa, Homo sapiens
YYSGS

SEQ ID NO: 355 - protein, 14 aa, Homo sapiens
LVYCGGDCYSGFDY

SEQ ID NO: 356 - protein, 11 aa, Homo sapiens
QASQDINNFLN

SEQ ID NO: 357 - protein, 7 aa, Homo sapiens
DASNLET

SEQ ID NO: 358 - protein, 9 aa, Homo sapiens
QQYGNLPFT

SEQ ID NO: 359 - protein, 9 aa, Homo sapiens
GGSISSGGF

SEQ ID NO: 360 - protein, 5 aa, Homo sapiens
HHSGS

SEQ ID NO: 361 - protein, 14 aa, Homo sapiens
LVYCGGDCYSGFDY

SEQ ID NO: 362 - protein, 11 aa, Homo sapiens
QASQDINNFLN

SEQ ID NO: 363 - protein, 7 aa, Homo sapiens
DASNLET

SEQ ID NO: 364 - protein, 9 aa, Homo sapiens
QQYGNLPFT

SEQ ID NO: 365 - protein, 15 aa, synthetic construct
GGGGSGGGGSGGGGS

SEQ ID NO: 366 - DNA, 1479 bp, synthetic construct
  source 1..1479: mol_type = other DNA
atgacccggctgacagtcctggccctgctggctggtctgctggcgtcctcgagggccgaggtgcagctggtgcagagcggcgccgaggtgaagaagcccggcagcagcgtgaaggtgagctgcaaggccagcggctacaccatcaccgacagcaacatccactgggtgcgccaggcccccggccagagcctggagtggatcggctacatctacccctacaacggcggcaccgactacaaccagaagttcaagaaccgcgccaccctgaccgtggacaaccccaccaacaccgcctacatggagctgagcagcctgcgcagcgaggacaccgccttctactactgcgtgaacggcaacccctggctggcctactggggccagggcaccctggtgaccgtgagcagcggtggaggcggttcaggcggaggtggcagcggcggtggcgggtcggacatccagctgacccagagccccagcaccctgagcgccagcgtgggcgaccgcgtgaccatcacctgccgcgccagcgagagcctggacaactacggcatccgcttcctgacctggttccagcagaagcccggcaaggcccccaagctgctgatgtacgccgccagcaaccagggcagcggcgtgcccagccgcttcagcggcagcggcagcggcaccgagttcaccctgaccatcagcagcctgcagcccgacgacttcgccacctactactgccagcagaccaaggaggtgccctggagcttcggccagggcaccaaggtggaggtgaaggagcccaagagctgcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgaccagcaagctgaccgtggacaagagccgctggcagcagggcaacgtgttcagctgcagcgtgatgcacgaggccctgcacaaccactacacccagaagagcctgagcctgagccccggcaag

SEQ ID NO: 367 - DNA, 1851 bp, synthetic construct
  source 1..1851: mol_type = other DNA
atgacccggctgacagtcctggccctgctggctggtctgctggcgtcctcgagggccgaggtgcagctggtggagagcggcggcggcctggtgcagcccggccgcagcctgcgcctgagctgcgccgccagcggcttcaccttcgacgactacagcatgcactgggtgcgccaggcccccggcaagggcctggagtgggtgagcggcatcagctggaacagcggcagcatcggctacgccgacagcgtgaagggccgcttcaccatcagccgcgacaacgccaagaacagcctgtacctgcagatgaacagcctgcgcgccgaggacaccgccctgtactactgcgccaagtacggcagcggctacggcaagttctactactacggcatggacgtgtggggccagggcaccaccgtgaccgtgagcgccgagttcaaggaggccttcagcctgttcgacaaggacggcgacggcaccatcaccaccaaggagctgggcaccgtgatgcgcagcctgggccagaaccccaccgaggccgagctgcaggacatgatcaacgaggtggacgccgacggcaacggcaccatcgacttccccgagttcctgaccatgatggcccgcaagatgaaggacaccgacagcgaggaggagatccgcgaggccttccgcgtgttcgacaaggacggcaacggctacatcagcgccgccgagctgcgccacgtgatgaccaacctgggcgagaagctgaccgacgaggaggtggacgagatgatccgcgaggccgacatcgacggcgacggccaggtgaactacgaggagttcgtgaagcagatcgtgctgacccagagccccgccatcatgagcgccagccccggcgagaaggtgaccatgacctgcagcgccagcagcagcgtgagctacatgaactggtaccagcagaagagcggcaccagccccaagcgctggatctacgacaccagcaagctggccagcggcgtgcccgcccacttccgcggcagcggcagcggcaccagctacagcctgaccatcagcggcatggaggccgaggacgccgccacctactactgccagcagtggagcagcaaccccttcaccttcggcagcggcaccaagctggagatcaaccgcgccgacgagcccaagagctgcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgtactgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggcaag

SEQ ID NO: 368 - DNA, 1851 bp, synthetic construct
  source 1..1851: mol_type = other DNA
atgacccggctgacagtcctggccctgctggctggtctgctggcgtcctcgagggccgaggtgcagctggtggagagcggcggcggcctggtgcagcccggccgcagcctgcgcctgagctgcgccgccagcggcttcaccttcgacgactacagcatgcactgggtgcgccaggcccccggcaagggcctggagtgggtgagcggcatcagctggaacagcggcagcatcggctacgccgacagcgtgaagggccgcttcaccatcagccgcgacaacgccaagaacagcctgtacctgcagatgaacagcctgcgcgccgaggacaccgccctgtactactgcgccaagtacggcagcggctacggcaagttctactactacggcatggacgtgtggggccagggcaccaccgtgaccgtgagcgccgagttcaaggaggccttcagcctgttcgacaaggacggcgacggcaccatcaccaccaaggagctgggcaccgtgatgcgcagcctgggccagaaccccaccgaggccgagctgcaggacatgatcaacgaggtggacgccgacggcaacggcaccatcgacttccccgagttcctgaccatgatggcccgcaagatgaaggacaccgacagcgaggaggagatccgcgaggccttccgcgtgttcgacaaggacggcaacggctacatcagcgccgccgagctgcgccacgtgatgaccaacctgggcgagaagctgaccgacgaggaggtggacgagatgatccgcgaggccgacatcgacggcgacggccaggtgaactacgaggagttcgtgcagcagatcgtgctgacccagagccccgccatcatgagcgccagccccggcgagaaggtgaccatgacctgcagcgccagcagcagcgtgagctacatgaactggtaccagcagaagagcggcaccagccccaagcgctggatctacgacaccagcaagctggccagcggcgtgcccgcccacttccgcggcagcggcagcggcaccagctacagcctgaccatcagcggcatggaggccgaggacgccgccacctactactgccagcagtggagcagcaaccccttcaccttcggcagcggcaccaagctggagatcaaccgcgccgacgagcccaagagctgcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgtactgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggcaag

SEQ ID NO: 369 - DNA, 1821 bp, synthetic construct
  source 1..1821: mol_type = other DNA
atgacccggctgacagtcctggccctgctggctggtctgctggcgtcctcgagggccgaggtgcagctggtggagagcggcggcggcctggtgcagcccggccgcagcctgcgcctgagctgcgccgccagcggcttcaccttcgacgactacagcatgcactgggtgcgccaggcccccggcaagggcctggagtgggtgagcggcatcagctggaacagcggcagcatcggctacgccgacagcgtgaagggccgcttcaccatcagccgcgacaacgccaagaacagcctgtacctgcagatgaacagcctgcgcgccgaggacaccgccctgtactactgcgccaagtacggcagcggctacggcaagttctactactacggcatggacgtgtggggccagggcaccaccgtgaccgtgagcgacaaggacggcgacggcaccatcaccaccaaggagctgggcaccgtgatgcgcagcctgggccagaaccccaccgaggccgagctgcaggacatgatcaacgaggtggacgccgacggcaacggcaccatcgacttccccgagttcctgaccatgatggcccgcaagatgaaggacaccgacagcgaggaggagatccgcgaggccttccgcgtgttcgacaaggacggcaacggctacatcagcgccgccgagctgcgccacgtgatgaccaacctgggcgagaagctgaccgacgaggaggtggacgagatgatccgcgaggccgacatcgacggcgacggccaggtgaactacgaggagttcgtgaagcagatcgtgctgacccagagccccgccatcatgagcgccagccccggcgagaaggtgaccatgacctgcagcgccagcagcagcgtgagctacatgaactggtaccagcagaagagcggcaccagccccaagcgctggatctacgacaccagcaagctggccagcggcgtgcccgcccacttccgcggcagcggcagcggcaccagctacagcctgaccatcagcggcatggaggccgaggacgccgccacctactactgccagcagtggagcagcaaccccttcaccttcggcagcggcaccaagctggagatcaaccgcgccgacgagcccaagagctgcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgtactgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggcaag

SEQ ID NO: 370 - DNA, 1860 bp, synthetic construct
  source 1..1860: mol_type = other DNA
atgacccggctgacagtcctggccctgctggctggtctgctggcgtcctcgagggccgaggtgcagctggtggagagcggcggcggcctggtgcagcccggccgcagcctgcgcctgagctgcgccgccagcggcttcaccttcgacgactacagcatgcactgggtgcgccaggcccccggcaagggcctggagtgggtgagcggcatcagctggaacagcggcagcatcggctacgccgacagcgtgaagggccgcttcaccatcagccgcgacaacgccaagaacagcctgtacctgcagatgaacagcctgcgcgccgaggacaccgccctgtactactgcgccaagtacggcagcggctacggcaagttctactactacggcatggacgtgtggggccagggcaccaccgtgaccgtgagcgccgagttcaaggaggccttcagcctgttcgacaaggacggcgacggcaccatcaccaccaaggagctgggcaccgtgatgcgcagcctgggccagaaccccaccgaggccgagctgcaggacatgatcaacgaggtggacgccgacggcaacggcaccatcgacttccccgagttcctgaccatgatggcccgcaagatgaaggacaccgacagcgaggaggagatccgcgaggccttccgcgtgttcgacaaggacggcaacggctacatcagcgccgccgagctgcgccacgtgatgaccaacctgggcgagaagctgaccgacgaggaggtggacgagatgatccgcgaggccgacatcgacggcgacggccaggtgaactacgaggagttcgtgcagatgagcggccagatcgtgctgacccagagccccgccatcatgagcgccagccccggcgagaaggtgaccatgacctgcagcgccagcagcagcgtgagctacatgaactggtaccagcagaagagcggcaccagccccaagcgctggatctacgacaccagcaagctggccagcggcgtgcccgcccacttccgcggcagcggcagcggcaccagctacagcctgaccatcagcggcatggaggccgaggacgccgccacctactactgccagcagtggagcagcaaccccttcaccttcggcagcggcaccaagctggagatcaaccgcgccgacgagcccaagagctgcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgtactgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggcaag

(19) United States
(12) Patent Application Publication
(10) Pub. No.: US 2025/0101104 A1
(43) Pub. Date: Mar. 27, 2025

(54) BISPECIFIC MOLECULE WITH TUNABLE AFFINITY TO A TARGETED ANTIGEN

(71) Applicant: MABSWITCH INC., Van Nuys, CA (US)

(72) Inventor: Ojo A. Onakunle, Van Nuys, CA (US)

(21) Appl. No.: 18/730,781

(22) PCT Filed: Jan. 25, 2023
(86) PCT No.: PCT/US23/61220

§ 371 Date: Jul. 21, 2024

Related U.S. Application Data
(60) Provisional application No. 63/303,212, filed on Jan. 26, 2022.

(51) Int. Cl. C07K 16/28

(52) U.S. Cl. CPC C07K 16/2809; C07K 16/2803; C07K 2317/31; C07K 2317/52; C07K 2317/622; C07K 2317/92

(57) ABSTRACT

The disclosure provides bispecific molecules that comprise a first binding domain comprising a VH and VL connected by an allosteric switch linker comprising a calmodulin polypeptide sequence, and a second binding domain that specifically binds an epitope different from that bound by the first binding domain; and wherein the first binding domain specifically binds or releases an antigen on the surface of an immune effector cell in the presence of Ca+ and a calmodulin binding ligand. Pharmaceutical compositions containing the bispecific molecules, nucleic acids encoding the bispecific molecules, host cells containing the nucleic acids and methods of making and using the bispecific molecules are also provided.

The present application is being filed accompanied by a Sequence Listing in electronic format. The Sequence Listing is provided as a file entitled 6942.0101_Sequence_Listing.txt, created Jan. 25, 2023, and which is 340 kilobytes in size. The information in the Sequence Listing is incorporated herein by reference in its entirety.

### FIELD

There is increasing interest in the use of bispecific antibodies as biologics drugs due, in large part, to the potential of achieving novel mechanisms of action that cannot be achieved with a combination of two conventional monospecific antibodies, or provide synergistic effects superior to the effect of individual antibodies. A lot of antibody engineering efforts have been put into designing new bispecific formats, such as DVD-Ig, CrossMab, BiTE etc. (Spiess et al., Mol. Immunol. 67(2):95-106 (2015)). Antibody-based therapies have proven effective treatments for several diseases but in some cases, toxicities have limited their therapeutic effectiveness. Bispecific molecules with tunable affinity to the target or to the effector cells can mitigate these problems and improve versatility of bispecific antibody treatments by limiting adverse effects and increasing safety.

### BACKGROUND

The selective destruction of an individual cell or a specific cell type is often desirable in a variety of clinical settings. For example, it is a primary goal of cancer therapy to specifically destroy tumor cells, while leaving healthy cells and tissues intact and undamaged. Upon recognition of a cancer antigen, detection of a microbial pathogen, or the presence of an allergen, an array of activating receptors and ligands induce the activation of the mammalian immune system. Such activation leads to the activation of macrophages, Natural Killer (NK) cells and antigen-specific, cytotoxic, T-cells, and promotes the release of various cytokines, all of which act to counter the perceived threat to the health of the subject. However, cancer cells can evade the immune reaction, which leads to tumor growth and life-threatening diseases. The disease state of cancer and indeed the disease states of infectious diseases therefore can be considered to reflect a failure to adequately activate a subject's immune system. Such failure may reflect an inadequate presentation of activating immune signals, or it may reflect an inadequate ability to alleviate inhibitory immune signals in the subject. An attractive way of correcting this inability is by inducing an immune response against the tumor by using a therapeutic molecule applied to the patient, to make immune effector cells such as natural killer (NK) cells or cytotoxic T lymphocytes (CTLs) attack and destroy tumor cells. CTLs constitute the most potent effector cells of the immune system, however they cannot be activated by the effector mechanism mediated by the Fc domain of conventional therapeutic antibodies. In this regard, bispecific antibodies designed to bind with one “arm” to a surface antigen on target cells, and with the second “arm” to an activating, invariant component of the T cell receptor (TCR) complex, have become of interest in recent years. The simultaneous binding of such an antibody to both of its targets will force a temporary interaction between target cell and T cell, causing activation of any cytotoxic T cell and subsequent lysis of the target cell. Hence, the immune response is re-directed to the target cells and is independent of peptide antigen presentation by the target cell or the specificity of the T cell as would be relevant for normal MHC-restricted activation of CTLs. A large variety of bispecific antibody formats have been developed, as known to those skilled in the art.

Several bispecific antibodies are approved for clinical use, as reviewed by Wang et al. (EMBO Mol. Med. (2021)13:e14291; doi 10.15252/emmm.202114291) or Salvaris et al. (J. Personalized Med. 11:355 (2021); doi.org/10.3390/jpm11050355) or Ma et al. (Front. Immunol. 12:626616 (2021); doi: 10.3389/fimmu. 2021.626616) and several of them target the T-cell antigen CD3 (cluster of differentiation 3). However, Immunotherapeutic anti-cancer agents, such as bispecific antibodies, in particular those binding to CD3, have an inherent risk of causing potentially fatal adverse effects, most notably cytokine release syndrome (CRS) (Salvaris et al., J. Pers. Med. 11:355 (2021); doi.org/10.3390/jpm11050355). For example, clinical safety concerns halted the trials of MGD011 and AFM11, illustrating the daunting challenge of managing CRS-related toxicity.

Any means to mitigate the risk of CRS and other adverse effects induced by overstimulating the immune system would help to increase safety of bispecific antibody treatments and therefore would generate a significant value for the development of future bispecific antibodies.

Calmodulin (CaM) undergoes large conformational changes, depending on the presence of calcium and calmodulin-binding peptides (CBP). In a calcium- and peptide-unbound form, it adopts a closed conformation (Kuboniwa et al., Nat. Struct. Biol. 2:768-776 (1995)). The distance between the N- and C-terminus is at its highest in the calcium-bound, open form (Chattopadhyaya et al., J. Mol. Biol. 228:1177-1192 (1992)), whereas the termini approach each other when calmodulin binds to a ligand, or a suitable fragment thereof, like peptide M13 (Ikura et al., Science 256:632-638 (1992).

Montigiani et al. (J. Mol. Biol. 258:6-13 (1996)) and Hultschig et al. (J. Mol. Biol. 343:559-568 (2004)) identified high affinity mutants of the CaM binding peptide “M13” which is derived from the rabbit myosin light chain kinase. There is a need in the art for an alternative or improved composition and/or method for affecting the binding of antigen-binding polypeptides to their respective antigens in the context of bispecific molecules.

### SUMMARY

In some embodiments, the present disclosure is directed to:
[1]A bispecific molecule, comprising:
   (a) a first binding domain comprising a variable heavy chain region (VH) and a variable light chain region (VL) covalently connected by an allosteric switch linker comprising a calmodulin polypeptide sequence or a fragment, variant or a cyclic mutant thereof, and
   (b) a second binding domain that specifically binds an epitope different from that bound by the first binding domain;
   wherein the first binding domain specifically binds an antigen on the surface of an immune effector cell in the presence of Ca+ and a calmodulin binding ligand;
[2] The bispecific molecule according to [1], wherein the immune effector cell is a T cell, a natural killer (NK) cell, a neutrophil, or a macrophage;
[3] The bispecific molecule according to [1] or [2], wherein the immune effector cell is a cytotoxic T lymphocyte;
[4] The bispecific molecule according to any one of [1]-[3], wherein the first binding domain specifically binds an antigen selected from CD3, CD28, CD137 (4-1BB), CD134 (0×40), CD27, ICOS the Vγ9 chain of the γδ T cell receptor, CD16A, NKG2C, NKG2D,NKp30a, NKp30b, NKp44, NKp46, 2DS1, 3DS1, 3DS2, 3DS4, 2DL5a, DNAM1, CD161, and CD47;
[5] The bispecific molecule according to any one of [1]-[4], wherein the first binding domain binds CD3, e.g., CD3epsilon (CD3ε);
[6] The bispecific molecule according to any one of [1]-[5], wherein the first and second binding domains are connected by one or more covalent bonds such as a peptide bond or a disulfide bond;
[7] The bispecific molecule according to any one of [1]-[6], wherein the bispecific molecule is a TriFab, Fab-Fv, Fab-dsFv, MAb-Fv, IgG-Fv, trivalent IgG-Fv (mAb-Fv) fusion protein, ScFab-Fc-scFv2, scFab-Fc-scFv, scFv-Fab IgG/XmAb, tandem scFv, diabody, (scFv)2-Fab, Fab-Fc, ScFv-Fc, DVD-Ig, IgG(H)-scFv or scFv-(H)IgG, IgG(L)-scFv or scFv-(L)IgG, 2scFv-IgG or IgG-2scFv, BITE, BITE-Fc, DART, DART-Fc Tetravalent DART, TandAb, scFv-scFv-scFv or an scFv-HSA-scFv;
[8] The bispecific molecule according to any one of [1]-[7], wherein the first binding domain comprises a switch linker and a VH and VL comprising:
   (a) an HCDR1 having the amino acid sequence of SEQ ID NO:301, an HCDR2 having the amino acid sequence of SEQ ID NO:302, an HCDR3 having the amino acid sequence of SEQ ID NO:303, an LCDR1 having the having the amino acid sequence of SEQ ID NO:304, an LCDR2 having the amino acid sequence of SEQ ID NO:305, and an LCDR3 having the amino acid sequence of SEQ ID NO:306;
   (b) an HCDR1 having the amino acid sequence of SEQ ID NO:307, an HCDR2 having the amino acid sequence of SEQ ID NO:308, an HCDR3 having the amino acid sequence of SEQ ID NO:309, an LCDR1 having the having the amino acid sequence of SEQ ID NO:310, an LCDR2 having the amino acid sequence of SEQ ID NO:311, and an LCDR3 having the amino acid sequence of SEQ ID NO:312;
   (c) an HCDR1 having the amino acid sequence of SEQ ID NO:313, an HCDR2 having the amino acid sequence of SEQ ID NO:314, an HCDR3 having the amino acid sequence of SEQ ID NO:315, an LCDR1 having the having the amino acid sequence of SEQ ID NO:316, an LCDR2 having the amino acid sequence of SEQ ID NO:317, and an LCDR3 having the amino acid sequence of SEQ ID NO:318;
   (d) an HCDR1 having the amino acid sequence of SEQ ID NO:319, an HCDR2 having the amino acid sequence of SEQ ID NO:320, an HCDR3 having the amino acid sequence of SEQ ID NO:321, an LCDR1 having the having the amino acid sequence of SEQ ID NO:322, an LCDR2 having the amino acid sequence of SEQ ID NO:323, and an LCDR3 having the amino acid sequence of SEQ ID NO:324;
   (e) an HCDR1 having the amino acid sequence of SEQ ID NO:325, an HCDR2 having the amino acid sequence of SEQ ID NO:326, an HCDR3 having the amino acid sequence of SEQ ID NO:327, an LCDR1 having the having the amino acid sequence of SEQ ID NO:328, an LCDR2 having the amino acid sequence of SEQ ID NO:329, and an LCDR3 having the sequence of SEQ ID NO:330; or
   (f) an HCDR1 having—the amino acid sequence of SEQ ID NO:331, an HCDR2 having the amino acid sequence of SEQ ID NO:332, an HCDR3 having the amino acid sequence of SEQ ID NO:333, an LCDR1 having the having the amino acid sequence of SEQ ID NO:334, an LCDR2 having the amino acid sequence of SEQ ID NO:335, and an LCDR3 having the amino acid sequence of SEQ ID NO:336; and the first binding domain specifically binds CD3 on the surface of an immune effector cell in the presence of Ca+ and a calmodulin binding ligand;
[9] The bispecific molecule according to any one of [1]-[8], wherein the first binding domain comprises a switch linker and a VH and VL comprising:
   (a) a VH having the amino acid sequence of SEQ ID NO:86 and a VL having the amino acid sequence of SEQ ID NO:87, or a humanized version thereof;
   (b) a VH having the amino acid sequence of SEQ ID NO:88 and a VL having the amino acid sequence of SEQ ID NO:89, or a humanized version thereof; and
   (c) a VH having the amino acid sequence of SEQ ID NO:90 and a VL having the amino acid sequence of SEQ ID NO:91, or a humanized version thereof;
   (d) a VH having the amino acid sequence of SEQ ID NO:288 and a VL having the amino acid sequence of SEQ ID NO:289, or a humanized version thereof;
   (e) a VH having the amino acid sequence of SEQ ID NO:290 and a VL having the amino acid sequence of SEQ ID NO:291, or a humanized version thereof;
   (f) a VH having the amino acid sequence of SEQ ID NO:292 and a VL having the amino acid sequence of SEQ ID NO:293, or a humanized version thereof;
   (g) a VH having the amino acid sequence of SEQ ID NO:294 and a VL having the amino acid sequence of SEQ ID NO:295, or a humanized version thereof; or
   (h) a VH having the amino acid sequence of SEQ ID NO:296 or 297 and a VL having the amino acid sequence of SEQ ID NO:298, 299, or 300;
   and the first binding domain specifically binds CD3 on the surface of an immune effector cell in the presence of Ca+ and a calmodulin binding ligand;
[10] The bispecific molecule according to any one of [1]-[9], wherein the second binding domain specifically binds a cell surface antigen expressed on the surface of a target cell;
[11] The bispecific molecule according to any one of [1]-[10], wherein the target cell is a cancer cell, an immune cell, a diseased cell, an infected cell, or a pathogen;
[12] The bispecific molecule according to any one of [1]-[11], wherein the second binding domain specifically binds a cell surface antigen expressed on the surface of a cancer cell;
[13] The bispecific molecule according to any one of [1]-[11], wherein the second binding domain has specific affinity for a cell surface antigen expressed on the surface of an immune cell;
[13] The bispecific molecule according to any one of [1]-[10] or [12], wherein the second binding domain has specific affinity for a cell surface antigen expressed on the surface of a diseased cell or an infected cell;
[14] The bispecific molecule according to any one of [1]-[10] or [11], wherein the second binding domain has specific affinity for a cell surface antigen expressed on the surface of a pathogen;
[15] The bispecific molecule according to any one of [1]-[15], wherein the second binding domain comprises a VH domain of an immunoglobulin and a VL domain of an immunoglobulin that is also covalently connected by an allosteric switch linker containing a calmodulin polypeptide sequence that specifically binds the epitope in the presence of Ca+ and a calmodulin binding ligand;
[16] The bispecific molecule according to any one of [1]-[12] or [15], wherein the second binding domain specifically binds a cell surface antigen selected from CD19, CD20, CD33, CD123, FLT3, CLL1, WT1, BCMA, GPRC5D, FcRH5, PSMA, DLL3, MUC1, MUC16, MUC17, EGFRviii, Mesothelin, STEAP-1, SSTR2, HER2, GPC3, GD2, TRAIL-R2, EPCAM, CLDN18.2;
[17] The bispecific molecule according to any one of [1]-[16], wherein the second binding domain comprises a VH and a VL that is also covalently connected by an allosteric switch linker containing a calmodulin polypeptide sequence that specifically binds the different epitope in the presence of Ca+ and a calmodulin binding ligand;
[18] The bispecific molecule according to any one of [1]-[17], wherein the allosteric switch linker comprises:
   (a) any one of the sequences of SEQ ID NO:92 to SEQ ID NO:287;
   (b) a fragment, variant, or a cyclic mutant of any one of the sequences of SEQ ID NO:92 to SEQ ID NO:287;
   (c) a sequence having at least 75%, 80%, 85%, 90% 95%, 96%, 97%, 98%, or 99% sequence identity with at least of the sequences of SEQ ID 92-287; or
   (d) a sequence that contains 1-25, 1-20, 1-15, 1-10, 1-5 or 5-20 amino acid additions, substitutions, and/or deletions compared to at least one of the sequences of SEQ ID 92-287;
[19] The bispecific molecule according to any one of [1]-[17], wherein the allosteric switch linker comprises any one of the sequences of SEQ ID NO:99 to SEQ ID NO:287;
[20] The bispecific molecule according to any one of [1]-[19], which further comprises a third binding domain;
[21] The bispecific molecule according to [20], wherein the third binding domain specifically binds:
   (a) an antigen conferring extended half-life to the bispecific molecule, such as human serum albumin or FcRn; or
   (b) a cell surface antigen expressed on the surface of a cancer cell, an immune cell, a diseased cell, an infected cell, or a pathogen;
[22] The bispecific molecule according to any one of [1]-[21], wherein the calmodulin binding ligand is selected from a member listed in Table 4 or a polypeptide comprising a sequence selected from SEQ ID NO:1 to SEQ ID NO:85;
[23]A pharmaceutical composition comprising the bispecific molecule according to any one of [1]-[22] and a pharmaceutically acceptable carrier;
[24]A pharmaceutical composition comprising the bispecific molecule according to any one of [1]-[22], a calmodulin binding ligand and a pharmaceutically acceptable carrier;
[25] An isolated nucleic acid or nucleic acids encoding the bispecific molecule according to any one of [1]-[22];
[26]A vector or vectors comprising the nucleic acid or nucleic acids of [25];
[27]A host cell comprising the nucleic acid or nucleic acids of [25] or the vector or vectors of [26];
[28]A method of making a bispecific molecule comprising culturing the host cell of [27] under conditions suitable for expression of the bispecific molecule, and optionally recovering the expressed bispecific molecule from the host cell or host cell culture medium;
[29] The pharmaceutical composition of [23] or [24], which is used for the treatment of cancer;
[30] Use of a bispecific molecule according to any one of the [1]-[21] or the pharmaceutical composition according to [23] or [24] in the treatment of cancer by administrating the bispecific molecule intravenously to the patient;
[31]A method of treating cancer comprising administering an effective amount of the bispecific molecule according to any one of [1]-[21] or the pharmaceutical composition according to [23] or [24] to an individual in need thereof;
[32] The method of [31] wherein the cancer is a hematological cancer;
[33] The method of [32] wherein the hematological cancer is selected from Non-Hodgkin Lymphoma, multiple myeloma, acute myeloid leukemia, Hodgkin's lymphoma, acute lymphoblastic leukemia, chronic lymphocytic leukemia, myelodysplastic syndromes, follicular lymphoma, chronic myelogenous leukemia, diffuse large B-cell lymphoma, cutaneous T-cell lymphoma, acute eosinophilic leukemia, anaplastic large cell lymphoma, Burkitt's lymphoma, and angioimmunoblastic T-cell lymphoma;
[34] The method of [31] wherein the cancer is a solid tumor cancer
[35] The method of [34] wherein the solid tumor cancer is selected from prostate cancer, breast cancer, lung cancer (non-small cell and small cell), colon cancer, rectal cancer, bladder cancer, pancreatic cancer, stomach (gastric) cancer, liver (hepatocellular) cancer, endometrial cancer, ovarian cancer, cervical cancer, esophageal cancer, head and neck cancer, oral cancer, melanoma, thyroid cancer, kidney (renal cell) cancer, testicular cancer, penile cancer, Anal cancer, retinoblastoma, uveal melanoma, merkel cell carcinoma, chondrosarcoma, Ewing's sarcoma, osteosarcoma, astrocytoma, glioblastoma, neuroblastoma, and mesothelioma;
[36]A method of treating a disease of the immune system comprising administering an effective amount of the bispecific molecule according to any one of [1]-[21] or the pharmaceutical composition according to [23] or [24] to an individual in need thereof;
[37] The method of [31], wherein the disease of the immune system is an inflammatory disease or an autoimmune disease such as rheumatoid arthritis;
[38]A method of treating an infectious diseases comprising administering an effective amount of the bispecific molecule according to any one of [1]-[21] or the pharmaceutical composition according to [23] or [24] to an individual in need thereof; or
[39]A kit comprising the bispecific molecule according to any one of [1]-[21] and a calmodulin binding ligand.

Additional embodiments of the provided bispecific molecules, nucleic acids, host cells, switch ligands and making and using these compositions are provided herein.

### BRIEF DESCRIPTION OF THE DRAWINGS

FIGS. 1A-1C. FIG. 1A: Principle of action of switchable bispecific compound. All elements are not drawn to scale, a, general function of bispecific antibodies. 1, Effector cell, typically a T-lymphocyte or natural killer cell; 2, target cell, typically a cancer cell or infected cell; 3, receptor on effector cell (1), typically an activating receptor like CD3 or CD16; 4, single chain polypeptide according to the disclosure, carrying the antigen binding domains (paratope) against the receptor (3) and comprising of two domains, typically the variable domain of the heavy chain and variable domain of the light chain of an antibody, and connected by the switch linker (5); 5, polypeptide carrying the switch element; 6, linker connecting (4) and (7), typically a polypeptide linker or a derivate of an antibody Fc region like SEED-body, knob-into-hole Fc, IgG4 Fc, or other structures that can connect (4) and (7); 7, binding molecule, typically an antibody, ligand or T-cell-receptor, that carries a binding site against a structure (8) on the target cell (2); 8, structure (8) on the target cell (2) which typically represents a tumor marker, or a cell surface protein like CD19; 9, signal induced by binding of (4) to (3); 10, pharmacological effect of the effector cell (1) induced by signal (9) to act on the target cell (2), typically cell killing (11) or inhibition of growth, signaling or differentiation. Dotted arrows indicate biological effects.

FIG. 1B: Principle of action of switchable bispecific compound according to the disclosure to inhibit the activity against the target cell (“off-switch”). 12, switch ligand; 13, allosteric effect of switch linker on single chain antibody upon switch ligand binding (right panel) decreases affinity for the effector receptor. Left panel, before switch ligand is added, right panel, after switch ligand is added. Bold arrow indicates allosteric switch step.

FIG. 1C: Principle of action of switchable bispecific compound according to the disclosure to increase the activity against the target cell (“on-switch”). 14, allosteric effect of switch linker on single chain antibody upon switch ligand binding increases affinity for the effector receptor. Left panel, before switch ligand is added, right panel, after switch ligand is added. Bold arrow indicates allosteric switch step.

FIG. 2. Examples for the many different ways to incorporate the disclosure into different constructs of bispecific antibodies. 1, target binding site of the target binding molecule, typically an antibody, ligand or T-cell-receptor, that carries a binding site against a structure on the target cell; 2, single chain polypeptide according to the disclosure, carrying the antigen binding domains (paratope) against the receptor of the effector cell and comprising of two domains, typically the variable domain of the heavy chain and variable domain of the light chain of an antibody, connected by the switch linker (11); 3, CH2-Region of Fc or other suitable linker; 4, Fab-region or other suitable linker; 5, CH3 regions of the Fc part of an antibody, engineered to promote selective heterologous dimerization to form bispecific antibodies with two different binding arms (1) and (2); 6, linker between the Fc regions and antigen recognition regions (typically a hinge region of an IgG); 7, Fc region; 8, target binding site in CH3, for example of a Fcab; 9, linker molecule improving the pharmacokinetic properties like serum half-life, typically human serum albumin or Fc region; 10, switch linker, 11, variable domains, 12, linker. Items labels apply to similar drawn elements throughout the figure. Elements are not drawn to scale.

FIG. 3. Some structures of bispecific molecules which comprise a scFv binding to CD3 as one of their targeting moieties. From Ma et al. (Front. Immunol. 12:626616 (2021); doi: 10.3389/fimmu.2021.626616).

FIG. 4. Schematic representation of the construction of the switchable bi-specific anti-CD33-CD3 scFv-Fc antibody according to the disclosure. 1, CD33 binding site of the cancer target binding scFv fragment derived from gemtuzumab; 2, CD3 binding site of the effector binding scFv fragment derived from OKT3, comprising of the variable domain of the heavy chain and variable domain of the light chain of the antibody, connected by the switch linker (3); 3, switch linker derived from human Calmodulin; 4, human IgG1 hinge region; 5, human IgG CH2 regions; 6, human CH3 regions of the Fc part of an antibody, engineered to promote selective heterologous dimerization to form bispecific antibodies with two different binding arms (1) and (2) by knob-into hole mutations, 7, variable region of the heavy chain of OKT3; 8, variable region of the light chain of OKT3; 9, variable region of the light chain of gemtuzumab; 10, variable region of the heavy chain of gemtuzumab. 11, Glycine-Serin Linker between the two variable regions of gemtuzumab (SEQ ID NO:365); 12, switch ligand (shown here in the bound configuration). Elements are not drawn to scale.

### DETAILED DESCRIPTION

The disclosure provides bispecific molecules that comprise a first binding domain comprising a variable heavy chain region (VH) and a variable light chain region (VL) covalently connected by an allosteric switch linker comprising a calmodulin polypeptide sequence, and a second binding domain that specifically binds an epitope different from that bound by the first binding domain; and wherein the first binding domain specifically binds or releases an antigen on the surface of an immune effector cell in the presence of Ca+ and a calmodulin binding ligand. In some embodiments, the immune effector cell is a T cell. In some embodiments the bispecific molecules have more than two binding specificities (e.g., are trispecific, tetraspecitic, pentaspecific, etc.). In some embodiments, the bispecific molecule further comprises a third binding domain. In some embodiments the second binding domain and/or third binding domain of the provided bispecific molecules specifically binds a cell surface antigen expressed on the surface of a target cell such as a cancer cell, an immune cell, a diseased cell, an infected cell, or the cell of a pathogen. Pharmaceutic compositions containing the provided bispecific molecules are also provided, as are nucleic acids encoding the bispecific molecules, host cells containing the nucleic acids and methods of making and using the provided bispecific molecules, pharmaceutical compositions, nucleic acids, and host cells. Further embodiments of the provided compositions and methods are provided herein.

### Definitions

It is to be noted that the term “a” or “an” entity refers to one or more of that entity; for example, “a bispecific antibody”, is understood to represent one or more bispecific antibodies. As such, the terms “a” (or “an”), “one or more”, and “at least one” can be used interchangeably herein.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers.

Throughout this application various publications are referred to in parentheses. Full citations for these references may be found at the end of the specification. The disclosures of these publications are hereby incorporated by reference in their entirety into the subject application to more fully describe the art to which the subject disclosure pertains.

Throughout this disclosure, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this disclosure to “one embodiment”, “an embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

Allosteric switch linker: this term is used interchangeably herein with “switch linker”.

Allosteric: “allosteric regulation”, “allosteric control” or “allosteric switch” is the regulation of a function of a molecule by binding or release of a second molecule, the “effector molecule” or “effector ligand” or “switch ligand” at a site other than that which provides the affected function. An example is the modulation of antigen binding by an antibody fragment that contains a switch linker.

Antibody: As used herein, an “antibody” or “antigen-binding polypeptide” or “monoclonal antibody” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. The term antibody encompasses various broad classes of polypeptides that can be distinguished biochemically. Those skilled in the art will appreciate that heavy chains are classified as gamma, mu, alpha, delta, or epsilon (γ, μ, α, δ, ε ν) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgG5, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region. Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule. Light chains are classified as either kappa or lambda (K, X). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CL domains actually comprise the carboxy-terminus of the heavy and light chain, respectively. In the case where there are two or more definitions of a term which is used and/or accepted within the art, the definition of the term as used herein is intended to include all such meanings unless explicitly stated to the contrary. Antibodies disclosed herein may be from any animal origin including birds and mammals. Preferably, the antibodies are human, murine, donkey, rabbit, goat, guinea pig, camel, llama, horse, or chicken antibodies, while completely human antibodies are particularly desirable for therapeutic treatment of human patients. In another embodiment, the variable region may be condricthoid in origin (e.g., from sharks). The antibodies may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art. The variable region of an antibody allows the antibody to selectively recognize and specifically bind epitopes on antigens. That is, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. More specifically, the antigen-binding site is defined by three CDRs on each of the VH and VL chains (i.e. CDR-H1, CDR-H2, CDR-H3, CDR-L1, CDR-L2 and CDR-L3). Antibodies suitable for treatment of human diseases can be generated by a large number of methods, including hybridoma technology, antibody phage display, humanization, chimerization, immunization of transgenic animals carrying human immunoglobulin gene loci and others, well established and known to those skilled in the art. The antibody may also be used in the form of an antibody-drug conjugate (ADC) obtained by binding of the antibody with an anticancer drug having tumor-cell proliferation inhibition efficacy. As used herein, the term “anticancer” includes “prevention” and “treatment” effects on cancer, and the “prevention” means any act of inhibiting or delaying cancer. In addition, the “treatment” means any act of ameliorating or beneficially altering symptoms of cancer.

Antibody fragment: The terms “antibody fragment” or “antigen-binding fragment”, as used herein may include, but are not limited to a portion of an antibody such as F(ab′)2, F(ab)2, Fab′, Fab, Fv, scFv and the like. Regardless of structure, an antibody fragment binds with the same antigen that is recognized by the intact antibody. The term “antibody fragment” includes a scFv, (scFv)2, sc(Fv)2, bispecific (scFv)2, bispecific sc(Fv)2, minibody, diabody, triabody and tetrabody.

Antigen: An “antigen” or “Ag” as used herein refers to a compound, composition, peptide, polypeptide, protein or substance that can stimulate the production of antibodies or a T cell or effector cell response in cell culture or in an animal, including compositions (such as one that includes a cancer-specific protein) that are added to a cell culture (such as a hybridoma), or injected or absorbed into an animal. An antigen reacts with the products of specific humoral or cellular immunity (such as an antibody), including those induced by heterologous antigens.

“TCR complex.” as used herein, refers to a complex formed by the association of CD3 chains with other TCR chains. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3γ chains, a TCRγ chain, and a TCRδ chain. TCR as used in the present disclosure may be from various animal species, including human, mouse, rat, or other mammals.

“A component of a TCR complex,” as used herein, refers to a TCR chain (e.g., TCRα, TCRβ, TCRγ or TCRδ), a CD3 chain (e.g., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3, or a sub-TCR complex of TCRα, TCRβ, CD3γ, CD3δ, and two CD3ε chains).

Biological effect: An effect which changes a parameter in a cell. In the context of this disclosure, it describes in particular an effect on the target cell that changes the fate of the target cell by inducing intracellular changes. In order to achieve a therapeutic effect and cure a disease or ameliorate disease symptoms, such biological effect, often can lead to the induction of apoptosis, inhibition of differentiation or the progression of the cell cycle, inhibition of cell division, or induction of cell death. Biological effects do not necessarily manifest as a single biochemical reaction, but rather are complex interactions of biological systems which lead to the final effect.

Bispecific reagent: In the context of this disclosure, “bispecific reagent”, bispecific molecule”, bispecific molecular entity” indicates in particular a molecular entity that can connect two other molecular entities which are not identical. One example are bispecific antibodies that are used in tumor therapy to connect effector cells to target cells. Bispecific antibodies may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art. Methods to constitute such conjugates are known by those skilled in the art.

Bispecific antibody: “bispecific antibody or “bispecific antibodies” describe antibodies or antibody-like molecules having two different binding specificities. This includes as well bispecific molecules with similar functions, e.g., bispecific constructs comprising an antibody arm and a ligand arm that binds to another target but is not originating from an immunoglobulin, e.g., a T cell receptor, a natural ligand like B7 or PD-L1 or other receptor binding domains. Bispecific antibodies have broad applications in biomedicine, especially in immunotherapy for tumors. Often, Bispecific antibodies are designed to target a tumor cell and an effector cell simultaneously, while triggering the effector cell's destruction of the tumor cell. Bispecific antibodies can be prepared by methods such as chemical engineering, cell engineering and genetic engineering. An advantage of genetic engineering is that the antibodies can be easily modified, which renders design and production of many different forms of bispecific antibody fragments, including diabodies, tandem ScFv, and single-chain diabodies, as well as derivatives thereof (reviewed by Jin and Zhu, in “the design and engineering of IgG-Like bispecific antibodies”, R E Kontermann (ed), Bispecific antibodies), while other bispecific antibody forms contain an Fc part or at least constant antibody domains. To improve the stability and therapeutic potential, recombinant genetic modifications were made in the heavy chains to facilitate their heterodimerization and to produce greater yields of Fc-containing IgG-like bispecific antibodies. Several rational design strategies have been used to engineer antibody CH3 chains for heterodimerizaiton, namely disulfide bonds, salt bridges, knobs-into-holes. Among these bispecific antibody formats, an IgG-like bispecific antibody is a common format: one arm binding to target A and another arm binding to target B. Structurally it is made from half of antibody A and half of antibody B, with the similar size and shape as natural IgG. In order to facilitate downstream development, it is desired that such bispecific molecules can be easily produced like a normal IgG from a single host cell with high expression level and correctly assembled form. Unfortunately, the pairing of cognate light-heavy chains as well as the assembly of two different half antibodies cannot be automatically controlled. All kinds of mispairings in a random manner could result in significant product heterogeneity. By introducing mutations in the Fc region, such as “knobs-into-holes” (Ridgway et al., Protein Eng. 9(7):617-621 (1996); Merchant et al., Nature Biotech. 16(7):677-681(1998)), electrostatics (Gunasekaran et al., J. Biol. Chem. 285(25):19637-19646 (2010)) or negative state designs (Kreudenstein et al., mAbs 5(5):646-654 (2013); Leaver-Fay et al., Structure, 24(4):641-651 (2016)), the preferred heterodimeric assembly of two different heavy chains has been accomplished. However, the selective pairing of light-heavy chains of each individual antibody remains challenging. The interface between light-heavy chains includes the variable domain (VH-VL) and the constant domain (CH1-CL). Several strategies have been applied into designing orthogonal interfaces to facilitate cognate pairing. Roche swapped the domains of CH1 and CL and created the CrossMab platform (Schaefer et al., Proc. Natl. Acad. Sci. (USA) 108(27):11187-11192 (2011)), MedImmune introduced alternatively a disulfide bond (Mazor et al., mAbs 7(2):377-389 (2015)), Amgen made further electrostatics in the CH1-CL region (Liu et al., J. Biol. Chem. 290(12):7535-7562 (2015)), and Lilly (Lewis et al., Nature Biotech. 32(2):191-198 (2014)) and Genentech (Dillon et al., mAbs, 9(2):213-230 (2017)) introduced mutations in both variable and constant domains.

Embodiments of bispecific antibodies include “TriFabs” which refers to a trivalent, bispecific fusion protein composed of three units with Fab-functionalities. TriFabs harbor two regular Fabs fused to an asymmetric Fab-like moiety. “Fab-Fv” refers to a fusion protein formed by fusing a VH to the C-terminus of a Fc chain and a VL to the C-terminus of a light chain. A “Fab-dsFv” molecule can be formed by introducing an interdomain disulfide bond between the VH domain and the VL domain. “MAb-Fv” or “IgG-Fv” refers to a fusion protein formed by fusion of VH domain to the C-terminus of one Fc chain and the VL domain either expressed separately or fused to the C-terminus of the other resulted in a bispecific, trivalent IgG-Fv (mAb-Fv) fusion protein, with the Fv stabilized by an interdomain disulfide bond. “scFab-Fc-scFv2” and “scFab-Fc-scFv” refer to a fusion protein formed by fusion of a single-chain Fab with Fc and disulfide-stabilized Fv domains. In some embodiments, the provided bispecific molecule is a TriFab, Fab-Fv, Fab-dsFv. MAb-Fv, IgG-Fv, trivalent IgG-Fv (mAb-Fv) fusion protein, ScFab-Fc-scFv2, scFab-Fc-scFv, scFv-Fab IgG/XmAb, tandem scFv, diabody, (scFv)2-Fab, Fab-Fc, ScFv-Fc, DVD-Ig, IgG(H)-scFv or scFv-(H)IgG, IgG(L)-scFv or scFv-(L)IgG, 2scFv-IgG or IgG-2scFv. BITE, BITE-Fc, DART, DART-Fc Tetravalent DART, TandAb, scFv-scFv-scFv or an scFv-HSA-scFv.

“Appended IgG” refers to a fusion protein with a Fab arm fused to an IgG to form the format of bispecific (Fab)2-Fc. It can form a “IgG-Fab” or a “Fab-IgG”, with a Fab fused to the C-terminus or N-terminus of an IgG molecule with or without a connector. In certain embodiments, the appended IgG can be further modified to a format of IgG-Fab4 (see, Brinkman et al., MABS 9(2):182-212 (2016); doi.org/10.1080/19420862.2016. 1268307). “DVD-Ig” refers to a dual-variable-domain antibody that is formed by fusion of an additional VH domain and VL domain of a second specificity to an IgG heavy chain and light chain. “CODV-Ig” refers to a related format where the two VH and two VL domains are linked in a way that allows crossover pairing of the variable VH-VL domains, which are arranged either (from N- to C-terminus) in the order VHA-VHB and VLB-VLA, or in the order VHB-VHA and VLA-VLB.A A “BiTE” or “Tandem antibody” is a bispecific T-cell engager molecule, comprising a first scFv with a first antigen specificity linked to a second scFv with a second specificity. Other embodiments of bispecific molecules are described by Ellerman (Methods (154):102-117 (2019);
doi.org/10.1016/j.ymeth.2018.10.026).

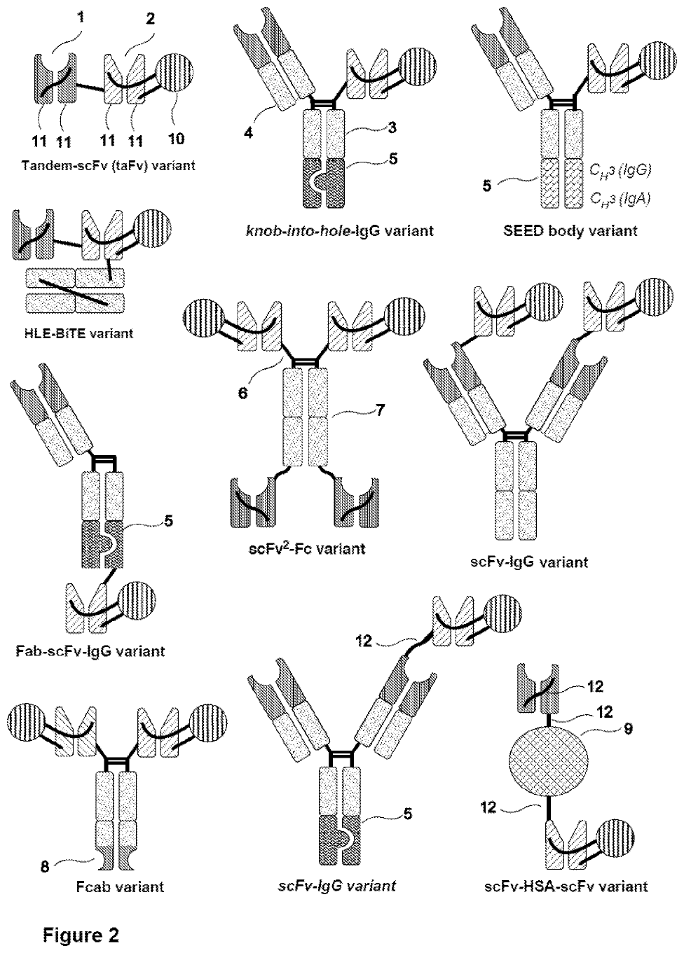

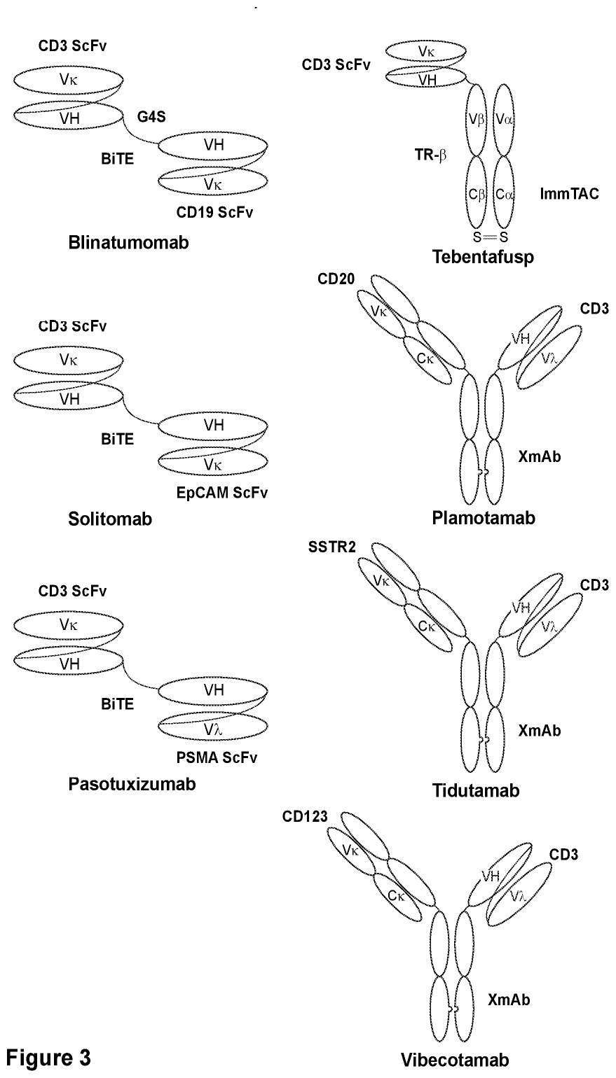

For clarity, the term “bispecific” in the context of this disclosure does not exclude molecules that have more than two specificities, so it includes molecules that have for example three or more specificities. Many bispecific antibodies have additional binding sites for binding to the FcRn receptor or FcGamma Receptors. The term “bispecific” therefore refers to molecular entities that comprise of at least (but not limiting) two different binding sites that represent a target binding moiety and an effector binding moiety. Effectors also can be other molecules, like complement components, acute phase proteins or other immunological effector mechanisms. For clarity, the term “bispecific” in the context of this disclosure does not exclude molecules that have more than two binding site per specificity, so it includes molecules that have for example three, four, five, six or seven or more binding sites with at least two different specificities. Many bispecific antibodies have two binding arms for the target, for example to gain from an avidity effect towards tumors with higher density of antigen versus normal cells, or to increase affinity to the target. Some embodiments of bispecific molecules with switch linkers are depicted in FIG. 2, depicting examples of possible variations in the format of the molecule which are all permissive to the regulation of their biological activity by switch ligands. FIG. 3 shows other formats which are currently in clinical use but are permissive to the addition of switch linkers according to the disclosure, allowing to improve their safety profile by regulation of their biological activity by switch ligands.

“Bispecific antibody therapy”, “T-cell engager therapy, “Immune cell engager therapy”, “antibody therapy” or “monoclonal antibody therapy” in the context of this disclosure are meant that the active pharmaceutical molecule which induces the therapeutic effect can be delivered as a purified polypeptide reagent, protein, chemically modified protein, but as well by gene therapy applications wherein it is delivered as mRNA or DNA such that the switch allows for the regulation of immune cell engager activity after the patient's cells have produced the bispecific antibody, T-cell or immune cell engager, for example genetically engineered T-cells “armored” to secrete switchable T-cell engager or other switchable immune cell engager or a genetically engineered natural killer cells “armored” to secrete switchable natural killer cell engager or other switchable immune cell engager at the site of the tumor. Calmodulin linker: linker containing a part of calmodulin or the full-length calmodulin protein, or a variant obtained by circular permutation, truncation, elongation or mutation. One embodiment is described in U.S. Pat. No. 10,730,922, the contents of which is hereby incorporated by reference in their entirety.

Calmodulin: a polypeptide with more than 74% sequence identity to human calmodulin, or a fragment of such polypeptide, or a circular mutation comprising calmodulin, or a a fragment of such circular mutated calmodulin. In some embodiments, a calmodulin polypeptide comprises a sequence having at least 75%, 80%, 85%, 90% 95%, 96%, 97%, 98%, or 99% sequence identity with the polypeptide sequence of SEQ ID 92-287.

Cancer cell: a cell that is part of a tumor that causes cancer. Cancer cells are the primary targets for cancer therapy.

CD3 (cluster of differentiation 3) “CD3” is a mu i-protein complex of six chains which are subunits of the T-cell receptor complex that is involved in activating both the cytotoxic T cell (CD8+ naive T cells) and T helper cells (CD4+ naive T cells). In mammals, the complex contains a CD3γ chain, a CD3δ chain, and two CD3ε chains. These chains associate with the T-cell receptor (TCR) and the CD3-zeta (ζ-chain) to generate an activation signal in T lymphocytes. The TCR, CD3-zeta, and the other CD3 molecules together constitute the TCR complex. Several clinically used bispecific antibodies were developed based upon CD3 binding antibody fragments to target CD3. CH2: “CH2” or “CH2 domain” as used herein refers to and includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat, et al., US DHHS (1983)).

CH3: The “CH3 domain” extends from the CH2 domain to the C-terminus of the IgG molecule and comprises approximately 108 amino acids. Certain immunoglobulin classes, e.g., IgM, further include a CH4 region.

CRS; cytokine release syndrome is a life-threatening adverse reaction that can occur during the treatment of patients with antibodies as reviewed by Shimabukuro-Vornhagen (J. Immunother. Cancer 6:56 (2018); doi: 10.1186/s40425-018-0343-9). Cytokine release syndrome (CRS) is a form of systemic inflammatory response syndrome (SIRS) that can be triggered by a variety of factors such as infections and certain drugs. It refers to cytokine storm syndromes (CSS) and occurs when large numbers of white blood cells are activated and release inflammatory cytokines, which in turn activate yet more white blood cells. CRS is an adverse effect of some monoclonal antibody medications, as well as adoptive T-cell therapies. When occurring as a result of a medication, it is also known as an infusion reaction. The term cytokine storm is often used interchangeably with CRS but, despite the fact that they have similar clinical phenotype, their characteristics are different. When occurring as a result of a therapy, CRS symptoms may be delayed until days or weeks after treatment. Immediate-onset CRS is a cytokine storm, although severe cases of CRS have also been called cytokine storms. CRS occurs when large numbers of white blood cells, including B cells, T cells, natural killer cells, macrophages, dendritic cells, and monocytes are activated and release inflammatory cytokines, which activate more white blood cells in a positive feedback loop of pathogenic inflammation. Immune cells are activated by stressed or infected cells through receptor-ligand interactions. Treatment for less severe CRS is supportive, addressing the symptoms like fever, muscle pain, or fatigue. Moderate CRS requires oxygen therapy and giving fluids and anti-hypotensive agents to raise blood pressure. For moderate to severe CRS, the use of immunosuppressive agents like corticosteroids may be necessary, but judgment must be used to avoid negating the effect of drugs intended to activate the immune system, With the success of T cell-engaging immunotherapeutic agents like bispecific antibodies, there has been a growing interest in CRS since it represents one of the most frequent serious adverse effects of these therapies. It was observed as a clinical problem of many bispecific antibodies, in particular those carrying a binding domain recognizing CD3 (Salvaris et al., J. Person. Med. 11:355 (2021))

Dimerization: combination of two molecular entities into one by covalent or non-covalent binding. In the context of this disclosure, it refers in particular to the combination of two different molecular entities to form a bispecific molecular entity.

Disulfide bond: “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system)

Domain: is a region of the protein's polypeptide chain that is self-stabilizing and that folds independently from the rest. Domains often form functional units, such as the calcium-binding EF hand domain of calmodulin. Because they are independently stable, domains can be “swapped” by genetic engineering between one protein and another to make chimeric proteins containing multiple domains that are not linked in the same way in nature. An example are many formats of bispecific antibodies.

Effector: a component of the human body that exerts a biological effect on a target upon recruitment by a bispecific reagent or bispecific antibody after those have bound to the target. Effectors can be immune cells or effector cells, or molecules of the defense against pathogens, for example complement components or molecules that regulate the immune response. An effector binding scFv is a scFv which specifically binds to the effector.

Effector cell: a cell that is used to increase the intended therapeutic effect of a bispecific construct versus the target cell. Examples for effector cells are T-lymphocytes, Treg cells, MAIT cells, macrophages or natural killer cells. An effector cell binding scFv is a scFv which specifically binds to the effector cell. In some embodiments, the effector cell is a T cell, a natural killer (NK) cell, a neutrophil, or a macrophage. In further embodiments, the effector cell is a cytotoxic T lymphocyte.

Effector receptor: molecule of the effector cell that can be targeted by a specific binder like a bispecific molecule and upon such binding induces an effect of the effector cell on the target cell. One example for effector receptor is the T-cell receptor complex.

Epitope: An “epitope” or “antigenic determinant” refers to the region of an antigen to which a binding agent (such as an antibody) binds. Epitopes can be formed both from contiguous amino acids (also called linear or sequential epitope) or noncontiguous amino acids juxtaposed by tertiary folding of a protein (also called configurational or conformational epitope). Epitopes formed from contiguous amino acids are typically arranged linearly along the primary amino acid residues on the protein, while conformational epitopes comprise of amino acid residues that are not assembled in a contiguous peptide stretch. An epitope typically includes at least 3, and more usually, at least 5, about 7, or about 8-10 amino acids in a unique spatial conformation.

Fab: “Fab” with regard to an antibody refers to that portion of the antibody consisting of a single light chain (both variable and constant regions) associating to the variable region and first constant region of a single heavy chain by a disulfide bond. In certain embodiments, the constant regions of both the light chain and heavy chain are replaced with TCR constant regions.

Fc: “Fc” with regard to an antibody refers to that portion of the antibody consisting of the second (CH2) and third (CH3) constant regions of a first heavy chain bound to the second and third constant regions of a second heavy chain via disulfide bonding. The Fc portion of the antibody is constituted by the joining of two heavy chains to form the typical Y-shaped immunoglobulin and provides various effector functions such as ADCC, and CDC, but does not function in antigen binding. In certain embodiments, the Fc Region of the Fc Region-containing molecules of the present disclosure may be an engineered variant Fc Region. Although the Fc Region of the bispecific Fc Region-containing molecules of the present disclosure may possess the ability to bind to one or more Fc receptors (e.g., FcγR(s)), more preferably such variant Fc Region have altered binding to FcγRIA (CD64), FcγRIIA (CD32A), FcγRIIB (CD32B), FcγRIIIA (CD16a) or FcγRIIIB (CD16b) (relative to the binding exhibited by a wild-type Fc Region), e.g., will have enhanced binding to an activating receptor and/or will have substantially reduced or no ability to bind to inhibitory receptor(s). Thus, the Fc Region of the Fc Region-containing molecules of the present disclosure may include some or all of the CH2 Domain and/or some or all of the CH3 Domain of a complete Fc Region, or may comprise a variant CH2 and/or a variant CH3 sequence (that may include, for example, one or more insertions and/or one or more deletions with respect to the CH2 or CH3 domains of a complete Fc Region). Such Fc Regions may comprise non-Fc polypeptide portions, or may comprise portions of non-naturally complete Fc Regions, or may comprise non-naturally occurring orientations of CH2 and/or CH3 Domains (such as, for example, two CH2 domains or two CH3 domains, or in the N-terminal to C-terminal direction, a CH3 Domain linked to a CH2 Domain, etc). Fc Region modifications identified as altering effector function are known in the art, including modifications that increase binding to activating receptors (e.g., FcγRIIA (CD16A) and reduce binding to inhibitory receptors (e.g., FcγRIIB (CD32B) (see, e.g., Stavenhagen et al., Cancer Res. 57(18):8882-8890 (2007)). In certain embodiments, it is preferred for the Fc Regions of bispecific molecules of the present disclosure to exhibit decreased (or substantially no) binding to FcγRIA (CD64), FcγRIIA (CD32A), FcγRIIB (CD32B), FcγRIIIA (CD16a) or FcγRIIIB (CD16b), or a combination of these. In certain embodiments of the present disclosure, the FcRn (neonatal Fc receptor) binding is increased by mutations within the Fc part to increase the plasma half-life of the bispecific molecule.

Fc parts can also carry modifications that allow for the formation of bispecific antibodies like knob-into-hole mutations, SEED-bodies or other mutations of the amino acid sequence that promote a heterologous association of two different molecular arms of a bispecific construct through such heterologous combination of the modified Fc parts, as described in more detail in the definition of bispecific antibodies.

In some embodiments, the Fc region of the long pharmaceutical half-life bi-specific molecule containing a switchable scFv with reduced or increased affinity for its target (e.g CD3) in the presence or absence of switch ligand, has been engineered to reduce FcγR and C1q binding in order to increase T-cell infiltration and anti-tumor activity to, and against solid tumors.

In certain embodiments, the Fc region of the long pharmaceutical half-life bi-specific molecule containing a switchable scFv with reduced or increased affinity for its target (e.g., CD3) in the presence or absence of switch ligand, has been engineered with N297A or N297A+K322A mutations to reduce FeγR and C1q binding in order to increase T-cell infiltration to and anti-tumor activity against solid tumors as described in Wang, L. et al., Cancer Immunol Res. 7(12): 2013-2024 (2019).

In some embodiments, the bi-specific molecule, with or without Fc or other half life extension moiety, containing a switchable scFv with reduced or increased affinity for its target (e.g., CD3) in the presence or absence of switch ligand is administered with, or following pre-medication of the patient with anti-inflammatory agents that modulate tumor infiltrating myeloid cells in order to enhance T-cell infiltration, persistence and anti-tumor activity against solid tumors.

In certain embodiments, the bi-specific molecule, with or without Fc or other half life extension moiety, containing a switchable scFv with reduced or increased affinity for its target (e.g., CD3) in the presence or absence of switch ligand is administered following pre-medication of the patient with dexamethasone in order to enhance T-cell infiltration, persistence and anti-tumor activity against solid tumors.

Hinge region: “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol. 161:4083-4090 (1998)).

Homology: “Homology” or “identity” or “similarity” or “homologous to” refers to sequence similarity between two polypeptides or between two nucleic acid molecules. Homology can be determined by comparing a position in each sequence which may be aligned for purposes of comparison. When a position in the compared sequence is occupied by the same base or amino acid, then the molecules are homologous at that position. As used herein, a “homologue sequence” and “homologous sequence” are used interchangeably and refer to polynucleotide sequences (or its complementary strand) or amino acid sequences that have sequence identity that can be quantified by given in % to another sequences when optionally aligned. A degree of homology between sequences is a function of the number of matching or homologous positions shared by the sequences. An “unrelated” or “non-homologous” sequence shares less than 40% identity with one of the sequences of the present disclosure. In certain embodiments, the derivative or variant is at least 75% the same as that of either the peptide or a fragment of the peptide having the same number of amino acid residues as the derivative. In certain embodiments, the derivative or variant is at least 85% the same as that of either the peptide or a fragment of the peptide having the same number of amino acid residues as the derivative. In certain embodiments, the amino acid sequence of the derivative is at least 90% the same as the peptide or a fragment of the peptide having the same number of amino acid residues as the derivative. In some embodiments, the amino acid sequence of the derivative is at least 95% the same as the peptide or a fragment of the peptide having the same number of amino acid residues as the derivative. In certain embodiments, the derivative or variant is at least 98% the same as that of either the peptide or a fragment of the peptide having the same number of amino acid residues as the derivative.

In some embodiments, a homologous polypeptide shares at least 75%, 80%, 85%, 90%, 95%, 96%, 97%, 98%, or 99% sequence identify with a reference sequence (e.g., VH, VL, calmodulin, or linker sequence provided herein, e.g., a sequence disclosed in Tables 1-3 (SEQ ID NO: 1 to SEQ ID NO: 370). “Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (PNAS USA 90(12):5873-5877 (1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option.

Immune Cell Engager: An example for Immune Cell Engagers are T cell-engagers. An example for T cell-engagers are BiTE molecules. Immune Cell Engagers are a well-established class of approved drugs and various methods for their generation and their design are known to those skilled in the art. Some examples are described by Goebeler and Bargou (Nat. Rev. Clin. Oncol. 17(7):418-434 (2020); doi: 10.1038/s41571-020-0347-5), or by Zhou et al. (Biomarker Research 9(38) (2021)), such as, but not limited to, bifunctional checkpoint-inhibitory T cell engagers (CiTEs), simultaneous multiple interaction T cell engagers (SMITEs), trispecific killer engagers (TriKEs) and BiTE-expressing chimeric antigen receptor (CAR) T cells, half-life extended BiTEs (HLE-BiTEs), Multispecific T cell engagers, innate or innate-like cell engagers, T-cell engagers with silenced IgG-Fc-domain, Multivalent T-cell engagers, as well as bispecific molecules targeting intracellular tumor-associated epitopes using bispecific constructs with T cell receptor (TCR)-derived, rather than an antibody-based, antigen-recognition domains, termed immune-mobilizing monoclonal TCRs against cancer (ImmTACs). In the context of this disclosure, for simplicity, the term “T-cell engager” subsumes bispecific molecules that engage effector cells and focusses their reactivity towards a target cell. This explicitly also includes bispecific reagents for the engagement of other cells than CD4+ or CD8+ T cells, Vγ9Vδ2 T cells or Treg cells like those binding to Natural Killer Cells as described by Demaria et al., Eur. J. Immunol. 51:1934-1942 (2021); doi: 10.1002/eji.2 02048953). Immune Cell Engagers as described in this disclosure can bind to the effector cell via different molecules and in the case of T cell binding bispecific reagents, this can be, but is not limited to, CD3 or the Vγ9 chain of the γδ T cell receptor, in case of natural killer cells, this favorably can be, but is not limited to, one of the following molecules: CD16A, NKp30, NKp46, NKG2D (natural killer group 2 member D) or other cell surface markers of other immune effector cells known to those skilled in the art. This binding in the context of this disclosure is provided by an effector binding scFv specific for said molecules.

Infection: infection is a transient or chronic viral, bacterial, fungal and parasitic infection.

“Inflammation”, “inflammatory disease” or “autoimmune disease” in the context of the present disclosure are defined by an adverse immune reaction that causes disease and is connected to the activation of the immune system. Inflammation is a biological response of the immune system that can be triggered by a variety of factors, including pathogens, damaged cells and toxic compounds. These factors may induce acute and/or chronic inflammatory responses in the heart, pancreas, liver, kidney, lung, brain, intestinal tract and reproductive system, potentially leading to tissue damage or disease. Many cases of inflammation are regulated by effector cells, in particular T cells or other effector cells. Autoimmune diseases are chronic inflammations which comprise a reactivity of a component of the immune system against a target in the patient's own body. This target can be a human antigen or protein but also members of our bodies microbiome. Inflammation is a common factor in many chronic diseases, including cardiovascular and bowel diseases, diabetes, arthritis, and cancer.

Ligand: molecular entity that can bind to a receptor. In the context of this disclosure, this indicates in particular proteins that bind to cell surface proteins of target cells, for example B7 that specifically binds to CD28, or PDL-1 that specifically binds to PD-1.

Linker: “link”, linked” and “linker” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity.

Molecular entity: a molecule which is acting as a single particle under physiological conditions. It can be an organic molecule, a polypeptide, a short peptide, or composed from different domains, linkers, regions and chemical links. A molecular entity can be constituted from the combination of two or more molecular entities, which are covalently or non-covalently linked.

Patient: The term “subject” or “individual” or “animal” or “patient” as used herein refers to human or non-human animal, including a mammal or a primate, in need of diagnosis, prognosis, amelioration, prevention and/or treatment of a disease or disorder. Mammalian subjects include humans, domestic animals, farm animals, and zoo, sports, or pet animals such as dogs, cats, guinea pigs, rabbits, rats, mice, horses, swine, cows, bears, and so on.

Polypeptide: As used herein, the term “polypeptide” is intended to encompass a singular “polypeptide” as well as plural “polypeptides”, and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein”, “amino acid chain”, or any other term used to refer to a chain or chains of two or more amino acids, are included within the definition of “polypeptide”, and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

Region: part of a macromolecule which can be defined as a structural or functional unit, for example a variable region of an immunoglobulin heavy chain, or an Fc region

Specificity: By “specifically binds” or “has specificity to”, it is generally meant that an ligand such as an antibody (e.g., an antigen binding fragment of an antibody) binds to an epitope via its antigen-binding domain, and that the binding entails some complementarity between the antigen-binding domain and the epitope. According to this definition, a ligand is said to “specifically bind” to an epitope, or an antigen containing the epitope, when it binds to that epitope, via its antigen-binding domain more readily than it would bind to a random, unrelated epitope. Thus, “specifically binds,” refers a ligand such as an antibody, or antigen binding fragment of an antibody, which recognizes and binds with a cognate binding partner/antigen (e.g., a stimulatory and/or costimulatory molecule present on a T cell) protein present in a sample, but which ligand does not substantially recognize or bind other molecules in the sample.

The term “specificity” is used herein to qualify the relative affinity by which a certain antibody binds to a certain epitope. For example, antibody “A” may be deemed to have a higher specificity for a given epitope than antibody “B”, or antibody “A” may be said to bind to epitope “C” with a higher specificity than it has for related epitope “D.” In some embodiments, the term “specific binding” or “specifically binds” as used herein refers to specifically binding of an antigen with a binding affinity (KD) of ≤10⁻⁶ M (e.g. ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤2×10⁻⁹ M, ≤10⁻⁹ M, or ≤10⁻¹⁰ M). In some embodiments, specific binding or specifically binds refers to specifically binding of an antigen with a binding affinity (KD) of 1×10⁻⁶ M to 5×10⁻⁹ M (e.g., 1×10⁻⁶ M to 5×10⁻⁸ M or 1×10⁻⁶ M to 5×10⁻⁷M). KD as used herein refers to the ratio of the dissociation rate to the association rate (koff/kon), may be determined using surface plasmon resonance methods or other methods well established in the field.

ScFv: A “single-chain variable fragment” or “scFv” refers to a fusion protein of the variable regions of the heavy (VH) and light chains (VL) of immunoglobulins. In some aspects, the regions are connected with a short linker peptide of ten to about 25 amino acids. The linker can either connect the N-terminus of the VH with the C-terminus of the VL, or vice versa. This protein retains the specificity of the original immunoglobulin, despite removal of the constant regions and the introduction of the linker. ScFv molecules are known in the art and are described, e.g., in U.S. Pat. No. 5,892,019.

Switch ligand: ligand binding to the allosteric switch linker with an affinity sufficient to induce an conformational change in the molecular entity that binds the switch ligand, Such conformational change affects the affinity of said molecular entity that binds the switch ligand to another, third molecule, so allowing to “influence”, “switch” or “tune” this affinity.

Switch linker: a linker allowing to “influence”, switch” or “tune” the affinity of binding of an antibody comprising a VH and VL to an antigen by an allosteric effect induced by the binding of a switch ligand. In one embodiment, the switch linkger

In one embodiment, the switch linker comprises a calmodulin derivative described in U.S. Pat. No. 10,730,922, the contents of which is hereby incorporated by reference in their entirety.

T-cell: “T cell” and “T-lymphocyte” as used herein refers to a type of lymphocyte that plays a critical role in the cell-mediated immunity, including but not limited to helper T cells (e.g., CD4+ T cells, T helper 1 type T cells, T helper 2 type T cells, T helper 3 type T cells, T helper 17 type T cells), cytotoxic T cells (e.g., CD8+ T cells), memory T cells (e.g., central memory T cells (TCM cells), effector memory T cells (TEM cells, Vγ9VS2 T cells, Treg cells and TEMRA cells) and resident memory T cells (TRM) that are either CD8+ or CD4+), natural killer T (NKT) cells and inhibitory T cells.

Variable regions: The terms “variable region” or “variable domain” with respect to an antibody as used herein refers to an antibody variable region or a fragment thereof comprising one or more CDRs. Although a variable domain may comprise an intact variable region (such as VH or VL), it is also possible to comprise less than an intact variable region yet still retain the capability of binding to an antigen or forming an antigen-binding site.

The present disclosure is related to a bispecific molecule (e.g., diabodies, bispecific antibodies, trivalent binding molecules, etc. as well as antibody-drug conjugates of such antibodies) that is characterized by having one binding specificity with an affinity to its antigen that can be regulated allosterically by the addition of a ligand, nucleic acid encoding the polypeptide, a method for the production of the antibody, the use thereof and pharmaceutical compositions comprising them. In particular, the binding arm that can be regulated is binding to the activating receptor of an effector cell, for example CD3. The regulation can be achieved separately from the administration of the bispecific molecule, by administration of a switch ligand, and this allows to regulate its effect over time. This regulation can either be achieved by administration of the switch ligand together with the bispecific reagent, with the regulation achieved by the quicker decay of plasma concentration of the switch ligand, or by administration of the switch ligand one or several times after the bispecific reagent, with the regulation achieved by the plasma level adjusted by these administrations. This allows to mitigate overshooting activity which may induce adverse reactions in the patient, or to switch on the activity sometime after the administration. This regulation is achieved by a component, the “switch ligand”, which can bind to a “switch linker” within the bispecific molecule, and upon binding allosterically influences the affinity of the binding of the bispecific molecule to the effector cell or molecule.

In one embodiment, the disclosure provides a bispecific molecule comprising a scFv that binds to CD3 and includes a switch linker allowing to modulate the affinity of said scFv for CD3 via a switch ligand.

Herein compositions comprising the above-mentioned molecular entities, methods for affecting the binding of the antigen by a scFv fragment carrying a switch linker, and the production of such molecules as well as their use in the treatment of various diseases are disclosed.

In some embodiments, the provided bispecific molecule comprises a switch linker described in U.S. Pat. No. 10,730,922 that describes a combination of a switch linker and a switch ligand to modulate the affinity of scFv fragments. The disclosure describes the use of said switch linker within a bispecific molecule. The contents of U.S. Pat. No. 10,730,922 are herein incorporated by reference in it entirety.

In some embodiments, the provided bispecific molecules comprise a switch linker containing a molecular entity that can allosterically affect the orientation of the variable fragment of the heavy chain of an antibody to the variable fragment of the light chain of an antibody in combination of a switch linker and a switch ligand to modulate the affinity of scFv fragments. The disclosure describes the use of said switch linker within a bispecific molecule.

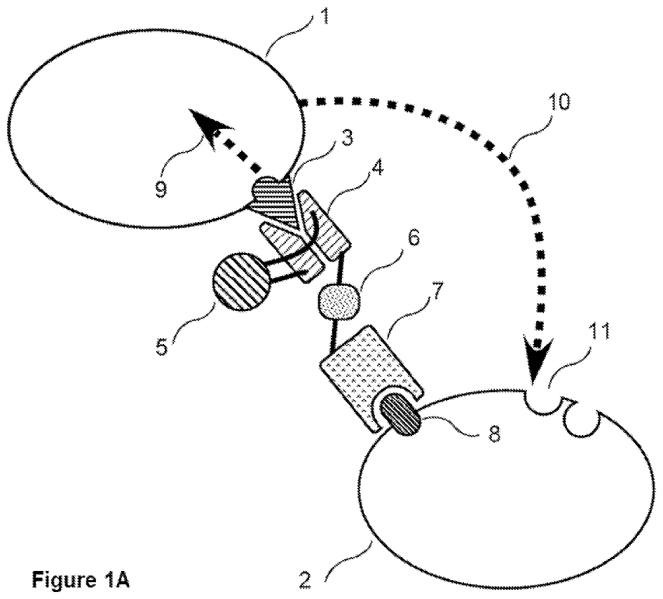

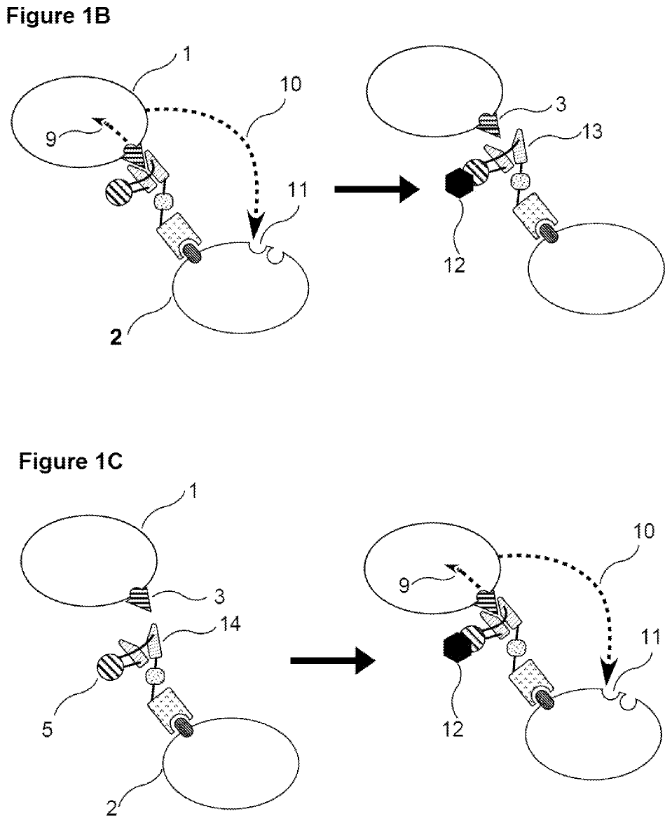

Within this bispecific molecule according to the disclosure that combines at least one binding arm to an antigen to another antigen which is typically a disease specific target, at least one of the other binding arms is a scFv fragment that carries the switch linker inserted between the variable domains (FIG. 1).

The disclosure describes the use of such bispecific molecule for the treatment of diseases. This is achieved by applying said bispecific molecule to the blood circulation of a patient. To regulate the activity of the biological effect within the patient, a second compound can be given to the patient. This compound, the switch ligand, binds to the switch linker that is included within the scFv fragments of the bispecific molecules, and thereby allosterically influences the affinity of said scFv fragments to their target or antigen.

In one embodiment, the binding of said switch ligand to the switch linker that is included within the scFv fragments of the bispecific molecules decreases the affinity of said scFv fragments to their target or antigen, as shown in FIG. 1b. By doing so, the addition of the switch linker induces a change of the strength of the biological effect that the bispecific molecule exerts onto its target in a way that is beneficial for the patient. In one embodiment, the bispecific molecule is an Immune Cell Engager. Here, the reduction of the affinity of the scFv fragment that binds to the T-cell or effector cell reduces the strength of the biological effect of the bispecific molecule, in particular the activation of the T cell or effector cell. The application of the switch linker thereby reduces the immunological activity of the T cell. This includes a decrease of the ability of the T cell or effector cell for direct killing of its target cell but also a decrease of the release of stimulating signals to the immune system, like cytokine secretion, or a decrease of the stimulation of cell differentiation or cell replication. In one embodiment, when the target cell is a cancer cell that is attacked by the T cell or effector cell, the addition of the switch linker therefore decreases the biological activity of the T cell or effector cell which binds to the bispecific molecule and avoids overshooting immune stimulation which can cause harm to the patient. The addition of the switch therefore allows to down-regulate the activity of the bispecific molecule, which in turn down-regulates the reactivity of the T cells or effector cell towards the tumor. This embodiment of the disclosure is employed to prevent overshooting immune stimulation, inflammation or sepsis after the application of the bispecific molecule to the patient. The application of the switch ligand to regulate the activity of said bispecific molecule ameliorates the overshooting immune stimulation.

Some embodiments of the disclosure describe scFv that are part of a bispecific molecule and that specifically bind to CD3. Some bispecific molecules that specifically bind to CD3 that are clinically evaluated are shown in FIG. 3. The addition of the switch linker to such bispecific molecules can provide them with the capability to be regulated after application to the patient, to prevent adverse effects like cytokine release syndrome, for the benefit of the patient. Here, they are switched off or downregulated by administration of the switch ligand to the patient to reduce the adverse effects and increase safety.

In one embodiment, wherein the binding of said switch ligand to the switch linker that is included within the scFv fragments of the bispecific molecules decreases the affinity of said scFv fragments of the bi-specific molecule to their target or antigen, said switch ligand is administered in sufficient quantity to the patient to effectively decrease binding of the scFv fragment to its target or antigen which may be a immune cell surface marker such as a CD3 antigen on a T-cell thus allowing the bi-specific molecule to accumulate within the diseased tissues prior to immune cell recruitment and activation that occurs as a result of switch ligand being pharmacokinetically cleared from the bloodstream.

In one embodiment, wherein the binding of said switch ligand to the switch linker that is included within the scFv fragments of the bispecific molecules decreases the affinity of said scFv fragments of the bi-specific molecule to their target or antigen which is a CD3 antigen on a T-cell, said switch ligand is administered in sufficient quantity to the patient to effectively delay binding of the scFv fragment to CD3 on T-cells to occur only following accumulation of the bispecific molecule in the tumor location in such a manner as to minimize the risk of antigen independent activation and antibody induced cell death (AICD) of T-cells as described by Wang et al., EMBO Mol. Med. 1-13: e14291 2021.

In one embodiment, the binding of said switch ligand to the switch linker that is included within the scFv fragments of the bispecific molecules increases the affinity of said scFv fragments to their target or antigen, as shown in FIG. 1C. By doing so, the addition of the switch linker induces a change of the strength of the biological effect that the bispecific molecule exerts onto its target that is beneficial for the patient. By doing so, the switch linker induces a change of the strength of the biological effect that the bispecific molecule exerts onto its target in a way that is beneficial for the patient. In one embodiment, the bispecific molecule is an Immune Cell Engager. Here, the increase of the affinity of the scFv fragment that binds to the T-cell or effector cell increases the strength of the biological effect of the bispecific molecule, in particular the activation of the T cell or effector cell. The application of the switch linker thereby increases the immunological activity of the T cell or effector cell. This includes an increase of the ability of the T cell or effector cell for direct killing of its target cell but also an increase of the release of stimulating signals to the immune system, like cytokine secretion, or an increase of the stimulation of cell differentiation or cell replication. In one embodiment, when the target cell is a cancer cell that is attacked by the T cell or effector cell, the addition of the switch linker therefore increases the biological activity of the T cell or effector cell which binds to the bispecific molecule. The addition of the switch therefore allows to up-regulate the activity of the bispecific molecule, which in turn up-regulate the reactivity of the T cells or effector cells towards the tumor or other suitable target. This embodiment of the disclosure is employed to induce immune stimulation against the target in a way that prevents overreactions which cause adverse effects for the patients, like inflammation or sepsis after the application of the bispecific molecule to the patient. In one embodiment, a bispecific molecule with a moderate or low affinity to the T cell or effector cell activating receptor is given to the patient when the tumor mass is large, to prevent an overshooting immune stimulation that can harm the patient while still being able to reduce the tumor mass, while switch ligand is added only or in increased amounts after the initial reduction of tumor mass to empower the T cells or effectors cells to be more aggressive against residual tumor cells. Some embodiments of the disclosure describe scFv that are part of a bispecific molecule that specifically binds to CD3. Some bispecific molecules that specifically bind to CD3 that are clinically evaluated are shown in FIG. 3. The addition of the switch linker to such bispecific molecules can provide them with the capability to be regulated after application to the patient, to prevent adverse effects like cytokine release syndrome, for the benefit of the patient. Here, they are switched on or upregulated by administration of the switch ligand to the patient, to adapt their activity to more effectively treat the disease.

In some embodiments, such bispecific molecules include, but are not limited to, bispecific molecules where an anti-CD3 or another effector binding scFv is combined or covalently linked with a binding molecule that specifically binds an antigen selected from: CD19, CD20, CD33, CD123, FLT3, CLL1, WT1, BCMA, GPRC5D, FcRH5, PSMA, DLL3, MUC1, MUC16, MUC17, EGFRviii, Mesothelin, STEAP-1, SSTR2, HER2, GPC3, GD2, TRAIL-R2, EPCAM, and CLDN18.2.

In some embodiments, the bispecific molecules comprises an anti-CD3 or another effector binding scFv that is combined or covalently linked with a binding molecule that specifically binds an antigen selected from CD1d, CD40, VEGF, PSCA, EGFR, WT1, CDH17, CD28, HER3, gamma-glutamyl-transferase (GGT), CEA, DLL1, STEAP1, B7, B7H6m, CTLA-4, gpA33, CD30, 5T4, CD137, CD138, SLAMF7, CS-1, CD319, LAG-3, CD52, VEGF-A, MASP-2, Tissue factor (TF), TROP-2, Nectin-4, CD79b, CD22, CCR4, PD-L1, PD1, a407 integrin, a4 integrin, PDGRFa, Glucocorticoid-induced TNFR-related protein (GITR), cMet (hepatocyte growth factor receptor), AXL, SIRPa, ICOS, 4-1BB, GUCY2c, Trop-2, Tumor-Restricted Ribonucleoprotein Complex, Oncofetal antigen 5T4, STEAP-1, CA125 (MUC16), ROR1, CD47, CD73, LRRC32, bCD32b, Globo H, CD228, TIGIT, Kallikrein-2, Mesothelin, Claudin-18.2, CD39, CD25, OX40, IL-27, CD45, CD47, CEACAM5, FGFR2b, Fibronectin extra-domain B, Folate receptor alpha, gp100, TIM-3 or other suitable cancer targets. The use of a switchable scFv within the bispecific molecule that induces the therapeutic effect of the effector cell against the target cell by a switch ligand that is administered sometime after the administration of the bispecific molecule allows for the enrichment of the bispecific molecule at the tumor site before it is activated, thereby increasing tumor specificity and decreasing unwanted off-target activity. In some embodiments, the scFv binding to CD3 is exchanged to an scFv binding to the Vγ9 chain of the γδ T cell receptor, NKG2D, CD16A, NKp30, NKG2D, CD47, NKp46 or another effector antigen to allow specificitiy to another type of effector cell.

In some embodiments, the scFv which contains a switch linker is part of one out of various molecular formats known to those skilled in the art to generate a bispecific molecule for therapeutic use. A large number of different molecular formats have been described for the generation of bispecific molecules, for example as described in Labrijn et al., (Nat. Rev. Drug Discov. 18:585-608 (2019); doi.org/10.1038/s41573-019-0028-1) or Ma et al., (Front. Immunol. (2021); doi.org/10.3389/fimmu.2021.626616) or Ellerman (Methods 154:102-117 (2019); doi.org/10.1016/j.ymeth.2018.10.026). Other molecular formats known to those skilled in the art can be used. Some molecular formats that can be used for the generation of a bispecific antibody according to this disclosure are shown in FIG. 2. Other molecular formats of bispecific molecules that are already in clinical use that can be modified by adding a switch linker to an antibody arm to make them regulatable are shown in FIG. 3. Indeed, the disclosure can be applied using all of the formats which contain a scFv antibody fragment, by adding the switch linker to said scFv fragments to add the capability for allosteric regulation to them.

In one embodiment, the bispecific antibody comprising the switch linker in the scFv that binds to the effector provides a secondary signal to the effector cell after target binding, to improve the activity when administered to the patient in combination with another bispecific reagent, for example a CD3×CD19 BiTE or another suitable bispecific immune cell engager. For example, NKG2D is also expressed by CD8+αβ T cells, γδ T cells, NKT cells as well as by subsets of CD4 T cells and mediates stimulating or co-activating signal priming, proliferation and function of cytotoxic T cells (Groh et al., Nat. Immunol. 2:255-260 (2001); and Maasho et al., J. Immunol. 174:4480-4484 (2005)). In one embodiment, scFv fragments binding to human killer cell immunoglobulin-like receptors (KIR), T cell-activated increased late expression (TACTILE), T cell immunoreceptor with Ig and ITIM domains (TIGIT), natural killer group 2 member A (NKG2A) or others can be used, as their blockade enhances NK-cell mediated antibody dependent cellular cytotoxicity (ADCC).

Bispecific Molecule Binding Domain that Specifically Binds an Immune Effector Cell

The provided bispecific molecule comprise a switchable first binding domain (i.e., a first binding domain comprising a variable heavy chain region (VH) and a variable light chain region (VL) covalently connected by an allosteric switch linker comprising a calmodulin polypeptide) that specifically binds an antigen on the surface of an immune effector cell in the presence of Ca+ and a calmodulin binding ligand. The Ca+ concentration does not have to be adjusted, as is determined in vivo by the physiological level present at the site of action of the bispecific molecule. The necessary concentration of the switch ligand is dependent on the desired switch level, but typically ranges between 10 nM-10 μM. The optimal concentration of the switch ligand further is determined individually for each combination of switch linkers and V regions of scFv fragments, as it has been observed that the same switch ligand can show different affinities to scFv constructs comprising the same switch linker by a factor of more than 7.2. Adding to this, variations of affinity of the Calmodulin to different switch ligands has been measured to vary between 2.2×10⁻¹² and 3.7×10⁻⁹ (Montigiani et al., J. Mol. Biol.; 258(1):6-13 (1996); doi: 10.1006/jmbi.1996.0229). The switch ligand/switch linker combination for each bispecific construct is therefore identified by testig combinations of the linkers shown in SEQ ID NO:92 to SEQ ID NO: 287 and the ligands shown by SEQ ID NO:1 to SEQ ID NO:85 or alternatiely the substances listed in Table 4, by determining the maximal switch factor (ratio between KD with and without switch ligand). The final dosing of the switch ligand is further depending on the concentration of the bispecific molecule applied to the patient, and adjusted typically to an at least 10 fold molar excess, typically a 30 fold, 50 fold 75 fold or 100 fold molar excess related to this molecule.

In some embodiments, the first binding domain of the provided bispecific molecule specifically binds an immune effector cell that is a T cell, a natural killer (NK) cell, a neutrophil, or a macrophage. In further embodiments, the bispecific molecule specially binds a cytotoxic T lymphocyte.

In some embodiments, the first binding domain of the provided bispecific molecule specifically binds an immune effector cell surface antigen selected from CD2, CD3, CD4, CD5, CD6, CD8, CD25, CD28, CD30, CD40, CD40L, CD44, CD45, CD69 and CD90.

In some embodiments, the first binding domain of the provided bispecific molecule specifically binds an immune effector cell surface antigen selected from CD137 (4-1BB), CLL-1, CD134 (0×40), CD27, ICOS the Vγ9 chain of the γδ T cell receptor, CD16A, NKG2C, NKp30, NKG2D, NKp30a, NKp30b, NKp44, NKp46, 2DS1, 3DS1, 3DS2, 3DS4, DNAM-1, CD16, CD161, and CD47.

In some embodiments, the first binding domain of the provided bispecific molecule specifically binds a TCR complex. In some embodiments, the first binding domain specifically binds a component of TCR complex. In further embodiments, the first binding domain specifically binds TCRα, TCRβ, TCRγ or TCRδ), a CD3 chain (e.g., CD37, CD3δ, CD3ε or CD3ζ), or a complex formed by two or more TCR chains or CD3 chains. In further embodiments, the specifically binds CD3ε.

In some embodiments, the first binding domain of the provided bispecific molecule specifically binds CD3 and comprises humanized VH and VL from an antibody selected from: muromonab-CD3 (OKT3), otelixizumab (TRX4), teplizumab (MGA031), visilizumab (Nuvion), SP34, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

OKT3 is a mouse-derived monoclonal IgG 2a-type antibody that recognizes the epitope of the F-subunit of the human CD3 complex (Kung et al., Science 206:347-349 (1979), Van Wauwe et al., J. Immunol. 124:2708-2713 (1980); Transy et al., Eur. J. Immunol. 19:947-950 (1989). In addition, the OKT3-producing hybridoma cell line has been published by the owner of European Patent No. 0018795 in the American Type Culture Collection (12301 Parklawn Drive, Rockville, Maryland, 20852) in the American Type Culture Collection ATCC No. CRL 8001, deposited on Apr. 26, 1979. In one embodiment, the antibody includes the point mutation at position H100A (according to the Kabat numbering system) in the amino acid sequence of OKT3 that increases stability many-fold, as described in (Kipriyanov et al., Protein Engineering 10:445-453 (1997)). The interaction of OKT3 with CD3 is described by Kjer-Nielsen et al., Proc. Natl. Acad. Sci. USA 101:7675-7680 (2004), and in Protein Data Base (PDB) file 15Y6.

In one embodiment, the switchable scFv contains the sequences homologous to that of mouse monoclonal antibody SP34 or an antibody derived from mouse monoclonal antibody SP34, where the antibody retains CDRs of the existing mouse anti-CD3 antibody, SP34, in which the amino acid sequence of the variable domain's framework regions of SP34 are partially or entirely substituted with a human framework counterpart sequence. In some embodiments, the switchable scFv comprises a VH and VL sequence comprising the amino acid sequence of SEQ ID NO:88 and SEQ ID NO:89, respectively. In some embodiments, the switchable scFv comprises a VH CDR1 comprising the sequence TYAMN (SEQ ID NO:307), a VH CDR2 comprising the sequence RIRSKYNNYATYYADSVKD (SEQ ID NO:308), a VH CDR3 comprising the sequence HGNFGNSYVSWFAY (SEQ ID NO:309), and a VL CDR1 comprising the sequence RSSTGAVTTSNYAN (SEQ ID NO:310), a VLCDR2 GTNKRAP (SEQ ID NO:311), and a VL CDR3 comprising the sequence ALWYSNLWV (SEQ ID NO:312).

In some embodiments, the antibody may be a humanized or human antibody version homologous to hybridoma derived monoclonal antibody UCHT1 or an antibody derived from monoclonal antibody UCHT1 (Kanellopoulos et al., EMBO J. 2(10):1807-1814 (1983)). The structure of UCHT1 is described in Proc. Natl. Acad. Sci. USA 101:16268-16273 (2004). In some embodiments, the scFv fragment contains VH and VL sequence comprising the amino acid sequence of SEQ ID NO:90 and SEQ ID NO:91, respectively. In some embodiments, the scFv comprises a VH CDR1 comprising the sequence GYTMN (SEQ ID NO:313), a VH CDR2 comprising the sequence LINPYKGVSTYNQKFKD (SEQ ID NO:314), a VH CDR3 comprising the sequence SGYYGDSDWYFDV (SEQ ID NO:315), and a VL CDR1 comprising the sequence RASQDIRNYLN (SEQ ID NO:316), a VL CDR2 comprising the sequence YTSRLHS (SEQ ID NO:317), and a VL CDR3 comprising the sequence QQGNTLPWT (SEQ ID NO:318).

In some embodiments, the antibody may be a humanized or human antibody that specifically binds to human CD3, typically to the CD3epsilon (CD3ε) component. The disclosure describes the use of these antibodies in particular, but not limited to, if their epitope is overlapping to the epitopes of OKT3, SP34 or UCHT1 or any of the other antibodies listed in SEQ ID NO:288 to SEQ ID NO:300.

In some embodiments, the disclosure provides a bispecific molecule corresponding to a known CD3 bispecific antibody wherein the VH and VL of the CD3 binding domain are engineered to be connected in an scFv format by introducing a switch linker provided herein between the VH and VL of the CD3 binding domain. In some embodiments, the CD3 bispecific molecule: specifically binds CD19 and comprises a binding domain comprising the VH and VL of blinatumomab (also known as AMG103 or MT103), or AFM11; specifically binds CD20 and comprises a binding domain comprising the VH and VL of ibritumomab, tositumomab, obinutuzumab, glofitamab, epcoritamab, rituximab, odronextamab, or Lymphomun FBTA05; specifically binds CD123 and comprises a binding domain comprising the VH and VL of flotetuzumab or MGD006; specifically binds BCMA and comprises a binding domain comprising the VH and VL of elranatamab, TNB-383B or REGN5458; specifically binds EpCAM and comprises a binding domain comprising the VH and VL of adecatumumab, catumaxomab, or MT110/AMG110; specifically binds CEA and comprises a binding domain comprising the VH and VL of MT111; specifically binds PSMA and comprises a binding domain comprising the VH and VL of AMG212, AMG160, HPN424, CCW702, CC-1 or MT112/BAY2010112; specifically binds GPA33 and comprises a binding domain comprising the VH and VL of MGD007; specifically binds DLL3 and comprises a binding domain comprising the VH and VL of tarlatamab; specifically binds GPRC5D and comprises a binding domain comprising the VH and VL of talquetamab; specifically binds Claudin 18.2 and comprises a binding domain comprising the VH and VL of AMG 910; specifically binds ErbB2 and comprises a binding domain comprising the VH and VL of ertumaxomab, GBR1302, M802, or RG6194; specifically binds Mucin 16 (MUC16) and comprises a binding domain comprising the VH and VL of REGN4018; or specifically binds GPC3 and comprises a binding domain comprising the VH and VL of ERY974.

In some embodiments, the affinity of the first binding domain of the bispecific molecule to the immune effector cell has a lower affinity, or is engineered for lower affinity before adding the switch linker, or this lower affinity has been achieved by adding a switch linker according to this disclosure.

In some embodiments, the antibody is a humanized or human antibody that specifically binds to human CD16A, NKp30, NKp46, CD56, NKG2D, NKp44, CD137 or CD244. As used herein, the term “humanized antibody” refers to an antibody that contains a minimal sequence derived from an immunoglobulin of a non-human antibody, such as a mouse antibody, and may mean such an antibody in which all parts except a sequence corresponding to a hypervariable region are substituted with their human counterparts. There are many methods to obtain humanized or human antibodies known to those skilled in the art. For the antibody, an antibody fragment thereof may also be used as long as the antibody fragment maintains the antibody's function.

In some embodiments, the first binding domain of the provided bispecific molecule comprises a switch linker and a VH and VL comprising:
(a) an HCDR1 having the amino acid sequence of SEQ ID NO:301, an HCDR2 having the amino acid sequence of SEQ ID NO:302, an HCDR3 having the amino acid sequence of SEQ ID NO:303, an LCDR1 having the having the amino acid sequence of SEQ ID NO:304, an LCDR2 having the amino acid sequence of SEQ ID NO:305, and an LCDR3 having the amino acid sequence of SEQ ID NO:306;
(b) an HCDR1 having the amino acid sequence of SEQ ID NO:307, an HCDR2 having the amino acid sequence of SEQ ID NO:308, an HCDR3 having the amino acid sequence of SEQ ID NO:309, an LCDR1 having the having the amino acid sequence of SEQ ID NO:310, an LCDR2 having the amino acid sequence of SEQ ID NO:311, and an LCDR3 having the amino acid sequence of SEQ ID NO:312;
(c) an HCDR1 having the amino acid sequence of SEQ ID NO:313, an HCDR2 having the amino acid sequence of SEQ ID NO:314, an HCDR3 having the amino acid sequence of SEQ ID NO:315, an LCDR1 having the having the amino acid sequence of SEQ ID NO:316, an LCDR2 having the amino acid sequence of SEQ ID NO:317, and an LCDR3 having the amino acid sequence of SEQ ID NO:318;
(d) an HCDR1 having the amino acid sequence of SEQ ID NO:319, an HCDR2 having the amino acid sequence of SEQ ID NO:320, an HCDR3 having the amino acid sequence of SEQ ID NO:321, an LCDR1 having the having the amino acid sequence of SEQ ID NO:322, an LCDR2 having the amino acid sequence of SEQ ID NO:323, and an LCDR3 having the amino acid sequence of SEQ ID NO:324;
(e) an HCDR1 having the amino acid sequence of SEQ ID NO:325, an HCDR2 having the amino acid sequence of SEQ ID NO:326, an HCDR3 having the amino acid sequence of SEQ ID NO:327, an LCDR1 having the having the amino acid sequence of SEQ ID NO:328, an LCDR2 having the amino acid sequence of SEQ ID NO:329, and an LCDR3 having the amino acid sequence of SEQ ID NO:330; or
(f) an HCDR1 having the amino acid sequence of SEQ ID NO:331, an HCDR2 having the amino acid sequence of SEQ ID NO:332, an HCDR3 having the amino acid sequence of SEQ ID NO:333, an LCDR1 having the having the amino acid sequence of SEQ ID NO:334, an LCDR2 having the amino acid sequence of SEQ ID NO:335, and an LCDR3 having the amino acid sequence of SEQ ID NO:336; and
the first binding domain specifically binds CD3 on the surface of an immune effector cell in the presence of Ca+ and a calmodulin binding ligand.

In some embodiments, the first binding domain of the provided bispecific molecule comprises a switch linker and a VH and VL comprising:
(a) a VH having the amino acid sequence of SEQ ID NO:86 and a VL having the amino acid sequence of SEQ ID NO:87, or a humanized version thereof;
(b) a VH having the amino acid sequence of SEQ ID NO:88 and a VL having the amino acid sequence of SEQ ID NO:89, or a humanized version thereof; and
(c) a VH having the amino acid sequence of SEQ ID NO:90 and a VL having the amino acid sequence of SEQ ID NO:91, or a humanized version thereof;
(d) a VH having the amino acid sequence of SEQ ID NO:288 and a VL having the amino acid sequence of SEQ ID NO:289, or a humanized version thereof;
(e) a VH having the amino acid sequence of SEQ ID NO:290 and a VL having the amino acid sequence of SEQ ID NO:291, or a humanized version thereof;
(f) a VH having the amino acid sequence of SEQ ID NO:292 and a VL having the amino acid sequence of SEQ ID NO:293, or a humanized version thereof;
(g) a VH having the amino acid sequence of SEQ ID NO:294 and a VL having the amino acid sequence of SEQ ID NO:295, or a humanized version thereof; or
(h) a VH having the amino acid sequence of SEQ ID NO:296 or 297 and a VL having the amino acid sequence of SEQ ID NO:298, 299, or 300;
and the first binding domain.

In some embodiments, the first binding domain of the provided bispecific molecule comprises a switch linker and a VH and VL comprising a sequence having at least 75%, 80%, 85%, 90% 95%, 96%, 97%, 98%, or 99% sequence identity with the VH and/or VL of any one of (a)-(h) in the immediately preceding paragraph, and specifically binds CD3 on the surface of an immune effector cell in the presence of Ca+ and a calmodulin binding ligand.

In some embodiments, the first binding domain of the provided bispecific molecule comprises a switch linker and comprises a VH and VL comprising an amino acid sequence that contains 1-25, 1-20, 1-15, 1-10, 1-5 or 5-20 amino acid additions, substitutions, and/or deletions compared to the VH and/or VL in any one of (a)-(h) above, and specifically binds CD3 on the surface of an immune effector cell in the presence of Ca+ and a calmodulin binding ligand.

In some embodiments, the first binding domain of the provided bispecific molecule comprises a switch linker and a VH and VL comprising a VHCDR1, VHCDR2, VHCDR3, VLCDR1, VLCDR2, and a VLCDR3 disclosed in Table 1 and specifically binds CD3 on the surface of an immune effector cell in the presence of Ca+ and a calmodulin binding ligand.

In some embodiments, the first binding domain of the provided bispecific molecule comprises a switch linker and a VH and VL disclosed in Table 1 and specifically binds CD3 on the surface of an immune effector cell in the presence of Ca+ and a calmodulin binding ligand. In some embodiments, the first binding domain of the provided bispecific molecule comprises a switch linker and a VH and VL comprising a sequence having at least 75%, 80%, 85%, 90% 95%, 96%, 97%, 98%, or 99% sequence identity with a VH and/or VL disclosed in Table 1 and specifically binds CD3 on the surface of an immune effector cell in the presence of Ca+ and a calmodulin binding ligand.

In some embodiments, the first binding domain of the provided bispecific molecule comprises a switch linker and a VH and VL comprising a sequence having an amino acid sequence that contains 1-25, 1-20, 1-15, 1-10, 1-5 or 5-20 amino acid additions, substitutions, and/or deletions compared to a VH and/or VL disclosed in Table 1 and specifically binds CD3 on the surface of an immune effector cell in the presence of Ca+ and a calmodulin binding ligand.

TABLE 1
Exemplary CD3 Binding Sequences

| ID | CDR Sequence (SEQ ID NO: ) | VH or VL Amino Acid Sequence (SEQ ID NO: ) |
|---|---|---|
| OKT3 | HCDR1: RYTMH (301) | EVQLVESGGGLVQPGRSLRLSCAASGFTFDDYSMHW |
| H | HCR2: YINPSRGYTNYNQKFKD (302) | VRQAPGKGLEWVSGISWNSGSIGYADSVKGRFTISRD |
| | HCDR3: YYDDHYCLDY (303) | NAKNSLYLQMNSLRAEDTALYYCAKYGSGYGKFYYYG |
| | | MDVWGQGTTVTVS (86) |
| OKT3 | LCDR1: SASSSVSYMN (304) | QIVLTQSPAIMSASPGEKVTMTCSASSSVSYMNWYQ |
| L | LCDR2: DTSKLAS (305) | QKSGTSPKRWIYDTSKLASGVPAHFRGSGSGTSYSLTIS |
| | LCDR3: QQWSSNPFT (306) | GMEAEDAATYYCQQWSSNPFTFGSGTKLEINRADTA |
| | | PTVSIFPPSSEQL (87) |
| SP34 | HCDR1: TYAMN (307) | EVQLVESGGGLVQPGGSLKLSCAASGFTFNTYAMNWV |
| H | HCR2: RIRSKYNNYATYYADSVKD (308) | RQAPGKGLEWVARIRSKYNNYATYYADSVKDRFTISRD |
| | HCDR3: HGNFGNSYVSWFAY (309) | DSQSILYLQMNNLKTEDTAMYYCVRHGNFGNSYVSWF |
| | | AYWGQGTLVTVSS (88) |
| SP34 | LCDR1: RSSTGAVTTSNYAN (3010) | QAVVTQESALTTSPGETVTLTCRSSTGAVTTSNYANW |
| L | LCR2: GTNKRAP (311) | VQEKPDHLFTGLIGGTNKRAPGVPARFSGSLIGGKAAL |
| | LCDR3: ALWYSNLWV (312) | TITGAQTEDEAIYFCALWYSNLWVFGGGTKLTVL (89) |
| UCHT1 | HCDR1: GYTMN (313) | EVQLQQSGPELVKPGASMKISCKASGYSFTGYTMNW |
| H | HCDR2: LINPYKGVSTYNQKFKD (314) | VKQSHGKNLEWMGLINPYKGVSTYNQKFKDKATLTV |
| | HCDR3: SGYYGDSDWYFDV (315) | DKSSSTAYMELLSLTSEDSAVYYCARSGYYGDSDWYFD |
| | | VWGQGTTLTVFS (90) |
| UCHT1 | LCDR1: RASQDIRNYLN (316) | DIQMTQTTSSLSASLGDRVTISCRASQDIRNYLNWYQ |
| L | LCR2: YTSRLHS (317) | QKPDGTVKLLIYYTSRLHSGVPSKFSGSGSGTDYSLTIS |
| | LCDR3: QQGNTLPWT (318) | NLEQEDIATYFCQQGNTLPWTFAGGTKLEIK (91) |
| 1H | HCDR1: DYSMH (319) | EVQLVESGGGLVQPGRSLRLSCAASGFTFDDYSMHW |
| | HDR2: GISWNSGSIGYADSVKG (320) | VRQAPGKGLEWVSGISWNSGSIGYADSVKGRFTISRD |
| | HCDR3: YGSGYGKFYYYGMDV (x321) | NAKNSLYLQMNSLRAEDTALYYCAKYGSGYGKFYYYG |
| | | MDVWGQGTTVTVS (288) |
| 1L | LCDR1: RASQSISSYLN (322) | DIQMTQSPSSLSASVGDRVTITCRASQSISSYLNWYQQK |
| | LCDR2: AASSLQS (323) | PGKAPKLLIYAASSLQSGVPSRFSGSGSGTDFTLTISSLQP |
| | LCDR3: QQSYSTPPIT (324) | EDFATYYCQQSYSTPPITFGQGTRLEIK (289) |
| 2H | HCDR1: KYAIN (325) | RQAPGKGLEWVARIRSKYNNYATYYADQVKDRFTISR |
| | HCDR2: RIRSKYNNYATYYADQVKD (326 | EVQLVESGGGLVQPGGSLKLSCAASGFTFNKYAINWV |
| | HCDR3: HANFGNSYISYWAY (327) | |
| | | DDSKNTAYLQMNNLTEDTAVYYCVRHANFGNSYISY |
| | | WAYWGQGTLVTVSS (290) |
| 2L | LCDR1: ASSTGAVTSGNYPN (328) | QTVVTQEPSLTVSPGGTVTLTCASSTGAVTSGNYPNWV |
| | LCDR2: GTKFLVP (329) | QQKPGQAPRGLIGGTKFLVPGTPARFSGSLLGGKAALTL |
| | LCDR3: TLWYSNRWV T (330) | SGVQPEDEAEYYCTLWYSNRWVFGGGTKLTVL (291) |
| 3H | HCDR1: AEVKKPGAYIH (331) | EVQLVQSGAEVKKPGAYIHWVRQAPGQGLEWIGWIY |
| | HCDR2: WIYPGDGNTKYNEKFKG (332) | PGDGNTKYNEKFKGRATLTADTSTSTAYLELSSLRSEDT |
| | HCDR3: DSYSNYYFDY (333) | AVYYCARDSYSNYYFDYWGQGTLVTVSS (292) |
| 3L | LCDR1: KSSQSLLNSRTRKNYLA (334) | DIVMTQSPDSLAVSLGERATINCKSSQSLLNSRTRKNYLA |
| | LCDR2: WASTRES (335) | WYQQKPGQPPKLLIYWASTRESGVPDRFSGSGSGTDFT |
| | LCDR3: TQSFILRT (336) | LTISSLQAEDVAVYYCTQSFILRTFGQGTKVEIK (293) |
| 4H | HCDR1: RSTMH (337) | QVQLVESGGGVVQPGRSLRLSCKASGYTFTRSTMHWV |
| | HCDR2: YINPSSAYTNYNQKFKD (338) | RQAPGQGLEWIGYINPSSAYTNYNQKFKDRFTISADKSK |
| | HCDR3: PQVHYDYNGFPY (339) | STAFLQMDSLRPEDTGVYFCARPQVHYDYNGFPYWGQ |
| | | GTPVTVSS (294) |
| 4L | LCDR1: SASSSVSYMN (340) | DIQMTQSPSSLSASVGDRVTMTCSASSSVSYMNWYQQ |
| | LCDR2: DSSKLAS (341) | KPGKAPKRWIYDSSKLASGVPARFSGSGSGTDYTLTISSL |
| | LCDR3: QQWSRNPPT (342) | QPEDFATYYCQQWSRNPPTFGGGTKLQIT (295) |
| 5Ha | HCDR1: RYTMH (343) | DVQLVQSGAEVKKPGASVKVSCKASGYTFTRYTMHWV |
| | HCDR2: YINPSRGYTNYADSVKG (344) | RQAPGQGLEWIGYINPSRGYTNYADSVKGRFTITTDKST |
| | HCDR3: YYDDHYCLDY (346) | STAYMELSSLRSEDTATYYCARYYDDHYCLDYWGQGTT |
| | | VTVSS(296) |
| 5Hb | HCDR1: RYTMH (343) | DVQLVQSGAEVKKPGASVKVSCKASGYTFTRYTMHWV |
| | HCDR2: YINPSRGYTNYNQKFKD (345) | RQAPGQGLEWIGYINPSRGYTNYNQKFKDRVTITTDKS |
| | HCDR3: YYDDHYCLDY (346) | TSTAYMELSSLRSEDTAVYYCARYYDDHYCLDYWGQGT |
| | | TVTVSS (297) |
| 5La | LCDR1: RASQSVSYMN (347) | DIQMTQSPSSLSASVGDRVTITCRASQSVSYMNWYQQ |
| | LCDR2: DTSKVAS (349) | KPGKAPKRWIYDTSKVASGVPARFSGSGSGTDYSLTINS |
| | LCDR3: QQWSSNPLTT (350) | LEAEDAATYYCQQWSSNPLTFGGGTKVEIK (298) |
| 5Lb | LCDR1: RASQSVSYMN (347) | DIVLTQSPATLSLSPGERATLSCRASQSVSYMNWYQQK |
| | LCDR2: DTSKVAS (349) | PGKAPKRWIYDTSKVASGVPARFSGSGSGTDYSLTINSL |
| | LCDR3: QQWSSNPLTT (350) | EAEDAATYYCQQWSSNPLTFGGGTKVEIK (299) |
| 5Lc | LCDR1: RASSSVSYMN (348) | DIVLTQSPATLSLSPGERATLTCRASSSVSYMNWYQQKP |
| | LCDR2: DTSKVAS (349) | GKAPKRWIYDTSKVASGVPARFSGSGSGTDYSLTINSLE |
| | LCDR3: QQWSSNPLTT (350) | AEDAATYYCQQWSSNPLTFGGGTKVEIK (300) |

Bispecific Molecule Domains that Specifically Bind an Antigen Expressed on the Surface of an Immune Cell, a Diseased Cell, an Infected Cell, or a Pathogen

The provided bispecific molecules comprise a switchable first binding domain (i.e., a first binding domain comprising a variable heavy chain region (VH) and a variable light chain region (VL) covalently connected by an allosteric switch linker comprising a calmodulin polypeptide as described herein) that specifically binds an antigen on the surface of an immune effector cell in the presence of Ca+ and a calmodulin binding ligand) and one or more additional binding domains that bind to an epitope that is different from that bound by the first binding domain. In some embodiments, the provided bispecific molecules comprise one or more binding domains that specifically bind an antigen that is overexpressed on the surface of a target cell. In some embodiments, the provided bispecific molecules comprise one or more binding domains that bind to a surface antigen on the surface of an immune cell. In some embodiments, the provided bispecific molecules comprise one or more binding domains that specifically bind an antigen that is overexpressed on the surface of diseased cell. In some embodiments, the diseased cell is a cancer cell. In some embodiments, the diseased cell is associated with a disorder of the immune system, such as an inflammatory disease or an autoimmune disease (e.g., rheumatoid arthritis or type 1 diabetes). In some embodiments, the diseased cell is a pathogen infected cell.

In some embodiments, the provided bispecific molecules comprise one or more binding domains that specifically bind an antigen that is expressed on the cell surface of a pathogen. In further embodiments, the pathogen is a viral, bacterial, fungal or parasitic pathogen.

In some embodiments, the second binding domain of the provided bispecific molecules is also switchable and comprises a VH and a VL covalently connected by an allosteric switch linker comprising a calmodulin polypeptide disclosed herein and specifically binds a target antigen in the presence of Ca+ and calmodulin. In some embodiments, one or more of the one or more additional binding domains in the provided bispecific molecules is also switchable and comprises a VH and a VL covalently connected by an allosteric switch linker comprising a calmodulin polypeptide disclosed herein and specifically binds a target antigen in the presence of Ca+ and calmodulin. These embodiments provide the ability to down-regulate overshooting activity of the bispecific molecule during treatment of for example, a cancer patient, by administering the switch ligand and thereby mitigating life-threatening adverse effects that frequently accompany CD3 bispecific antibody administration such as CRS.

In some embodiments, the one or more additional binding domains contained in the provided bispecific molecules (e.g., the “second binding domain” and/or one or more further additional binding domains) binds an antigen expressed on the cell surface of a tumor or cancer cell. In some embodiments, the second binding domain and/or another binding domain of the provided bispecific molecule specifically binds a cell surface antigen expressed on the surface of a hematological cancer cell. In some embodiments, the second binding domain and/or another binding domain specifically binds a cell surface antigen expressed on the surface of a solid tumor cell.

In some embodiments, the second binding domain and/or one or more further additional binding domains specifically bind an antigen selected from: CD19, CD20, CD33, CD123, FLT3, CLL1, WT1, BCMA, GPRC5D, FcRH5, PSMA, DLL3, MUC1, MUC16, MUC17, EGFRviii, Mesothelin, STEAP-1, SSTR2, HER2, GPC3, GD2, TRAIL-R2, EPCAM, and CLDN18.2.

In some embodiments, the second binding domain and/or one or more further additional binding domains specifically bind an antigen selected from: CD1d, CD40, CD19, VEGF, EGFRviii, PSCA, FLT3, EGFR, CD33, CD20, CLEC12A, WT1, CD123, FLT3, DLL3, CDH17, BCMA, CD28, EpCAM, GPRC5D, HER2, HER3, MUC1, gamma-glutamyl-transferase (GGT), PSMA, CEA, MUC17, DLL1, STEAP1, B7, B7H6, gpA33, GD2, CD30, 5T4, SSTR2, CD137, CD138, SLAMF7, CS-1, CD319, CD38, LAG3, CD52, CD25, VEGF-A, MASP-2, Tissue factor (TF), TROP2, Nectin-4, CD79b, CD22, CCR4, PD-L1, PD1, a407 integrin, a4 integrin, CTLA-4, PDGRFa, Glucocorticoid-induced TNFR-related protein (GITR), cMet (hepatocyte growth factor receptor), AXL, SIRPa, ICOS, 4-1BB, GUCY2c, Tumor-Restricted Ribonucleoprotein Complex, Oncofetal antigen 5T4, CA125 (MUC16), ROR1, CD47, CD73, LRRC32, CD32b, Globo H, CD228, TIGIT, Kallikrein-2, Mesothelin, Claudin-18.2, CD39, OX40, IL-27, CD38, CD45, CD47, CEACAM5, FGFR2b, Fibronectin extra-domain B, Folate receptor alpha, gp100, and TIM-3.

In some embodiments, the second binding domain and/or one or more further additional binding domains of the provide bispecific molecule specifically binds an antigen selected from: HER2-neu, HER4, MUC2, MUC3, MUC4, MUC-5ac, MUC5B, Wue-1, Plasma Cell Antigen (see Intl. Publ. No. WO 01/47953), (membrane-bound) IgE, TNFRSF17, IL3RA, SDC1, MS4A1, sTn (sialylated Tn antigen), FAP (fibroblast activation antigen), Igo, MT-MMPs, Cora antigen, L6, CO-29, CD7, CD22, Igα (CD79a), G250 (CA9), F19-antigen, 9-O-Acetyl-GD3, ganglioside GD3, GM2, fucosyl GM1, poly sialic acid, D44v6, Sonic Hedgehog (Shh), Melanoma Chondroitin Sulfate Proteoglycan (MCSP), CCR8, TNF-alpha precursor, STEAP2, A33 Antigen, Ly-6, desmoglein 4, E-cadherin neoepitope, Fetal Acetylcholine Receptor, CA19-9 marker, Muellerian Inhibitory Substance (MIS), Receptor type II, endosialin, SAS, CD63, TAG72, TF-antigen, CD5, NCAM1, CD70, NKG2DL (ULBP1), NKG2DL2 (ULBP2), ILiRAP, GPC3, PDCD1, CD274, KDR, IL13RA2, LlCAM, CD23, CD44 (e.g., CD44v6), CD174, GPNMB, CD276, CSPG4, CD133, RON, CEACAM-6, PCTA-1, PSA, PAP, ALCAM (CD166), PECAM-1, CD151, MAGE-1, IGF1R, TGFBR2, GHRHR, GHR, IL-6R, gp130, TNFRSFlB (TNFR2), OSMRP, Patched-1, Frizzled, Robol, LTOR, CD26, TNFRSF7 (CD27), CD44, CD80 CD81, CD86, CD100, CXCR4, CCR5, MUC7, PhCG, Lewis-Y, LG, TNFRSF12 (TWEAKR), FGFR4, VEGFR1, VEGFR2, SSX1, and SSX2, and TEM1.

In some embodiments, the disclosure provides a bispecific molecule wherein the second binding domain and/or one or more additional binding domains comprises a VH and VL of a known antibody sequence. In some embodiments, the second binding domain and/or other binding domain of the bispecific molecule: specifically binds glycoprotein IIb/IIIa and comprises a binding domain comprising the VH and VL of abciximab; specifically binds CD52 and comprises a binding domain comprising the VH and VL of alemtuzumab; specifically binds CD33 and comprises a binding domain comprising the VH and VL of gemtuzumab; specifically binds EGFR and comprises a binding domain comprising the VH and VL of cetuximab or panitumumab; specifically binds ErbB2 and comprises a binding domain comprising the VH and VL of trastuzumab, ertumaxomab, GBR1302, M802, or RG6194; specifically binds PD-1 receptor and comprises a binding domain comprising the VH and VL of lambrolizumab or nivolumab; specifically binds IL-6 receptor and comprises a binding domain comprising the VH and VL of atlizumab or tocilizumab; specifically binds CA125 and comprises a binding domain comprising the VH and VL of abagovomab or benralizumab; specifically binds CTLA-4 and comprises a binding domain comprising the VH and VL of ipilimumab; specifically binds CD25 and comprises a binding domain comprising the VH and VL of basiliximab or daclizumab; specifically binds CD11a and comprises a binding domain comprising the VH and VL of efalizumab; or specifically binds a4 integrin and comprises a binding domain comprising the VH and VL of atalizumab.

In some embodiments, the second binding domain and/or other binding domain of the bispecific molecule: specifically binds glycoprotein IIb/IIIa and comprises a binding domain comprising the VH and VL of abciximab;

In some embodiments, the second binding domain and/or one or more further additional binding domains of the provide bispecific molecule specifically binds CD19. Bispecific molecules provided herein that specifically bind CD19 are useful in treating diseases such as a proliferative disease, a tumorous disease, an inflammatory disease, an immunological disorder, an autoimmune disease, an infectious disease, a viral disease, allergic reactions, parasitic reactions, graft-versus-host diseases, host-versus-graft diseases or B-cell malignancies, in particular B cell non-Hodgkin's lymphoma, Hodgkin's lymphoma or B cell leukemias (e.g. B-ALL, pre-B-ALL, hairy cell lymphoma, and B-CLL).

In some embodiments, the disclosure describes a bispecific molecule for the treatment of the hematological malignancy acute myeloid leukemia (AML). In some embodiments, the AML is AML with at least one genetic abnormality, AML with multilineage dysplasia, therapy-related AML, undifferentiated AML, AML with minimal maturation, AML with maturation, acute myelomonocytic leukemia, acute monocytic leukemia, acute erythroid leukemia, acute megakaryoblastic leukemia, acute basophilic leukemia, acute panmyelosis with fibrosis or myeloid sarcoma. There are different well knnown disease related targets which can be employed to guide the bispecific molecule to the tumor. Bispecific antibodies represent a promising immunotherapeutic approach for the treatment of AML (Guy et al., Curr. Hematol. Malig. Rep. 13(6):417-425 (2018); doi: 10.1007/s11899-018-0472-8).

In some embodiments, the disease related target located on AML cells is CD33. In this embodiment, the bispecific switchable T cell engager of this disclosure targets CD33. Relevant CD33 variants are identified by the NCBI Reference Sequence: NM_001772.4, NM_001177608.2 and NM_001082618.2. CD33, also known as sialic acid-binding Ig-like lectin 3 (Siglec-3) is a 67 kDa trans-membrane cell surface glycoprotein, which is commonly expressed on acute myeloid leukemia blasts in >90% of patients as well as leukemic stem and progenitor cells. It is estimated that about 30% of healthy bone marrow myeloid progenitors express CD33, which leads to on-target off-tumor toxicity. Multiple therapeutic concepts have targeted CD33 since the early 2000s including monoclonal antibodies, antibody-drug conjugates and retargeting of T-cell with bispecific molecules. The anti-CD33 antibody-drug conjugate gemtuzumab ozogamicin (Mylotarg) was initially approved for treatment of relapsed AML in 2000, later withdrawn from the market after the failure of a large randomized phase III study conducted in the US and then reapproved in 2017 following the results of four European randomized studies which demonstrated survival benefit for favorable and intermediate risk AML patients.

In some embodiments, the variable domains of Gemtuzumab (SEQ ID NO:351 and SEQ ID NO:352) are used to generate the cancer-specific arm of the bispecific antibody. Gemtuzumab is a recombinant humanized IgG4 kappa antibody. The antibody is specifically directed against the CD33 antigen present on leukemic myeloblasts in most patients with acute myeloid leukemia (AML). A Antibody drug conjugate consisting of Gemtuzumab as targeting entity has achieved marketing approval against AML (gemtuzumab ozogamicin). On Sep. 1 2017, gemtuzumab ozogamicin was approved for the treatment of adults with newly diagnosed CD33-positive acute myeloid leukemia. It is also indicated for the treatment of patients aged 2 years and older with CD33-positive AML who have experienced a relapse or who have not responded to initial treatment (refractory). (Egan et al., Onco. Targets Ther. 11:8265-8272 (2018): 10.2147/OTT.S150807)

VL of gemtuzumab
(SEQ ID NO: 351)
DIQLTQSPSTLSASVGDRVTITCRASESLDNYGIRFLTWFQQKPGKAPK
LLMYAASNQGSGVPSRFSGSGSGTEFTLTISSLQPDDFATYYCQQTKEV
PWSFGQGTKVEVK
VH of gemtuzumab
(SEQ ID NO: 352)
EVQLVQSGAEVKKPGSSVKVSCKASGYTITDSNIHWVRQAPGQSLEWIG
YIYPYNGGTDYNQKFKNRATLTVDNPTNTAYMELSSLRSEDTAFYYCVN
GNPWLAYWGQGTLVTVSS

In some embodiments, clinically used bispecific antibodies targeting CD33 are employed that have already been constructed to treat AML. In one embodiment, a bispecific molecule is constructed by exchanging the linker connecting VH and VL of the CD3 targeting scFv fragment within AMG330 by a switch linker. AMG330 is a human BiTE tandem single-chain antibody with the N-terminal specific for human CD33 and its C-terminal is directed towards CD3ε. In vitro studies showed activation of T-cells manifested as expression of CD69 and CD25 as well as release of IFN-7, TNF-α Interleukin-2, IL-10 and IL-6. Ex vivo, AMG330 showed a potent dose- and effector to target cell ratio-dependent activity against human AML cell lines. Daily intravenous administration of AMG330 significantly prolonged the survival of immune-deficient mice adoptively transferred with human MOLM-13 AML cells and human T-cells. AMG330 was approved for a first in human phase I study with continuous intravenous infusion in patients with relapsed/refractory AML. Alternatively, a similar switchable bispecific molecule can be built based on AMG673, a related anti-CD33×anti-CD3 BiTE antibody construct. AMG673 fuses an Fc domain extending the half-life of the antibody to 7 days which permits weekly dosing of the agent, but also results in a slower decrease of adverse effects, allowing a larger benefit from a switchable anti-tumor activity introduced by the addition of the functionality of a switchable linker as disclosed herein.

In another embodiment, a bispecific molecule is constructed by exchanging the linker connecting VH and VL of the CD3 targeting scFv fragment within AMV564 by a switch linker. AMV564 is a tetravalent anti-CD33×anti-CD3 tandem diabody construct that is comprised of two VH and VL chains that form antigen-binding single chain variable fragments (scFvs). The protein forms a homodimer creating two binding sites for each epitope and increases the avidity of the antibody to its targets. It also increases the molecular weight to approximately 106 kDa avoiding first pass renal clearance and resulting in a longer half-life in comparison to BiTEs. Preclinical data with cryopreserved human AML specimens demonstrated in vitro activity across the spectrum of disease stage, cytogenetic risk and CD33 expression levels. AMV564 was approved to be tested in a first in human phase I trial in patients with relapsed or refractory AML in which the agent is dosed as a 14 day continuous infusion every 28 days. Results presented at the European Hematology Association in June 2018 showed evidence of T-cell activation evident by increased cytokine levels and antigen markers of T-cell activation. There was evidence of biologic activity with 13-38% reductions in the bone marrow blasts in 10/16 patients.

In another embodiment, a bispecific molecule is constructed by exchanging the linker connecting VH and VL of the CD3 targeting scFv fragment within GEM333 by a switch linker. GEM333 is another CD3×CD33 humanized bispecific antibody tested in Phase I clinical trials for relapsed or refractory AML. GEM333 antibody is a humanized single chain bispecific antibody with variable and light and heavy chains to both CD3 and CD33 are arranged in a tandem format using a novel linker. In preclinical studies, the construct was capable of redirecting human T cells efficiently toward CD33+AML blasts as demonstrated by killing of AML cell lines and patient-derived AML blasts both in vitro and in NSG mice. Notably, the antibody had no effect on normal human CD34+ hematopoietic stem and progenitor cells in both colony forming assays or in NSG repopulating experiments.

In addition to cytotoxic T-cells, there is an established role for NK cells in the exertion of graft versus leukemia effect. Clinical data show that alloreactive NK cell from mismatched stem cell grafts are associated with a potent GVL effect. Haploidentical NK cell infusions have been shown to improve outcomes in patients with refractory AML. CD16 is a potent NK cell activating receptor that showed promising results as an effector target for the treatment of lymphoid tumors. A new anti-CD33×CD16 bi-specific killer cell engager (BiKE) was created (Wiernik et al., Clin Cancer Res. 15; 19(14)(2013) July:3844-55; doi:10.1158/1078-0432.CCR-13-0505). This BiKE was shown to induce NK cell activation and degranulation in response to CD33+HL60 cells. It also induced target cell death as measured by chromium release assay in all E:T ratios. The BiKE induced NK cell activation against AML blasts from a patients with de-novo AML. Addition of IL-15 crosslinker to promote NK cell activation and proliferation, termed a trispecific killer engager (TriKE) improves NK function against AML cell lines. In some embodiments, the scFv fragment targeting the biological effector cell is composed of the VH and VL regions of an antibody against CD16, and between these variable antibody region, a switch linker is inserted to regulate the activation of Natural Killer (NK) cells.

In one embodiment, the bispecific switchable T cell engager of this disclosure targets CD123. CD123 (Interleukin-3 receptor alpha chain) is the low affinity binding subunit of the IL3 receptor. CD123 is expressed on myeloid progenitors, plasmacytoid dendritic cells, monocytes and basophils. Binding of IL-3 triggers CD123 heterodimerization with the β-subunit shared by the granulocyte macrophage-colony-stimulating-factor and IL5 receptor complex inducing hematopoietic progenitor cell differentiation and proliferation by phosphorylation of Janus kinase, activation of PI3 kinase and upregulation of anti-apoptotic proteins. CD123 was initially described as a putative marker of leukemic stem cells with increased expression in the CD34+/CD38-primitive leukemic blasts which contained the NSG repopulating activity of AML samples. Elevated expression of CD123 on AML blasts is associated with higher blasts counts and poorer prognosis with lower CR rates. Early studies with anti-CD123 antibodies showed that inhibiting CD123 led to prolonged survival of AML xenografted mice.

In one embodiment, a bispecific molecule is constructed by exchanging the linker connecting VH and VL of the CD3 targeting scFv fragment within Flotetuzumab by a switch linker. Flotetuzumab (MGD-006, Macrogenics) is a CD3×CD123 DART that was shown to recognize CD123⁺ leukemia cells and induce T-cell activation with resulting cytotoxic effect. MGDO06 induced a dose-dependent killing of AML cell lines and primary AML blasts in vitro and in NOD SCID7 mice injected with human AML.

In one embodiment, a bispecific molecule is constructed by exchanging the linker connecting VH and VL of the CD3 targeting scFv fragment within construct analogous to JNJ-63709178 by a switch linker. JNJ-63709178 is a CD3×CD123 bispecific IgG1 antibody generated using a process called controlled Fab-arm exchange also known as Genmab DuoBody® technology. The method involves the separate expression of two parental IgG1 monoclonal antibodies that each contain a single matched point mutation at the CH3-CH3 domain interface, ere, the CD3 targeting antigen binding site is replaced with a scFv fragment of the same specificity. During controlled reduction of disulfide bonds in which the parental antibodies separate into half molecules, the matched CH3 mutation drive the reassembly of heterozygous binding arms producing the bispecific IgG1 antibody. In contrast to BiTE's and DARTs, these IgG like bispecific antibodies retain their Fc region and its associated effector functions and in vivo stability. The antibody was shown to recruit T-cells to CD123 expressing tumor cells inducing cytolytic effect in vitro. In AML murine xenograft models, the compound was able to suppress tumor growth and induce tumor regression in the presence of human peripheral blood T-cells. A phase I clinical trial in relapsed and refractory AML patients was initiated but placed on hold in March 2016 due to grade 3 events. No further clinical information has been released by the sponsor, illustrating the high need to tune the activity of the bispecific antibody within the patient, using the switch linker and switch ligands as disclosed herein to improve this construct.

In one embodiment, a bispecific molecule is constructed by exchanging the linker connecting VH and VL of the CD3 targeting scFv fragment within a construct analogous to XmAb14045 by a switch linker. XmAb14045 possesses a unique Fc domain and spontaneously forms stable heterodimers to facilitate manufacturing of the antibody. The Fc domain was also engineered to abolish binding to FcγR to reduce the potential for nonselective T cell activation but preserve binding to human FcRn and maintain long serum half-life. In contrast to non-Fc domain-containing bispecific formats XmAb14045 had a prolonged serum half-life in mice of 6.2 days. In vitro, XmAb14045 stimulated T cell-mediated killing of CD123+KG-1a and TF-1 AML cell lines with an EC50<1 ng/ml (8 μM). In cynomolgus monkeys, XmAb14045 strongly activated T cells and stimulated depletion of over 99% of circulating CD123⁺ cells including basophils and plasmacytoid dendritic cells within 1 hour of administration.

In one embodiment, the bispecific switchable T cell engager of this disclosure targets CLEC12A, also named CLL-1, shows a high expression on AML cells while being absent in normal hematopoietic stem cells. The CLL-1 sequence is listed at uniprot.org/uniprot/Q5QGZ9, as well as NCBI Reference Sequence NP_612210.4; ncbi.nlm.nih.gov/protein/NP 612210.4). As used herein, CLL-1 includes human CLL-1 and non-human CLL-1 homologs, as well as variants, fragments, or post-transnationally modified forms thereof, including, but not limited to, N- and O-linked glycosylated forms of CLL-1.

In one embodiment, a bispecific molecule is constructed by exchanging the linker connecting VH and VL of the CD3 targeting scFv fragment within a construct analogous to MCLA-117 by a switch linker. MCLA-117 is a human full length IgG1 bispecific antibody targeting CLEC12A (also known as CLL-1) and CD3. CLEC12A is a myeloid differentiation antigen expressed on ˜90% of newly diagnosed and relapsed AML. CLEC12A is selectively expressed on leukemic stem cells, but not on normal early hematopoietic progenitors including hematopoietic stem cells. The antibody arm targeting CD3 within MCLA-117 was designed with a low affinity anti-CD3 Fab arm for controlled T-cell activation, and is replaced by an anti CD3 scFv fragment with switchable affinity as disclosed herein. This combines with the tumor targeting antibody arm as well as high affinity anti-CLEC12A arm for efficient tumor targeting. It also encompasses a silenced Fc region to prevent binding to FcγR+ cells while retaining a long half-life. Analysis of normal bone marrow demonstrated that MCLA-117 binds to the granulocyte-macrophage progenitor, however not to the CD34⁺CD38⁻ compartment that includes hematopoietic stem cells. In vitro studies showed efficient CLEC12A antigen dependent T-cell activation and tumor target cell lysis in HL-60 cell line, as well as normal monocytes. MCLA-117 was able to induce T-cell mediated lysis of AML blasts in an ex vivo culture system, despite low effective effector-to-target (E:T) ratios.

In an embodiment, the tumor targeting arm of the switchable bispecific effector cell enganger consists of an antigen binding molecule that specifically binds to CLL-1, wherein the antigen binding molecule is a single chain variable fragment (scFv) comprising a variable heavy chain (VH) comprising CDR1, CDR2 and CDR3 according to SEQ ID NOS: 353, 354 and 355, respectively, and a variable light chain (VL) comprising CDR1, CDR2 and CDR3 according to SEQ ID NOS: 356, 357 and 358, respectively. The methods for the generation of such scFv and the DNA encoding the respective variable regions are fully disclosed in EP. Pat. No. EP3436030B1. In some embodiments, the provided bispecific molecule comprises a VHCDR1 comprising the sequence GGSISSY (SEQ ID NO:353); a VHCDR2 comprising the sequence YYSGS (SEQ ID NO:354); a VHCDR3 comprising the sequence LVYCGGDCYSGFDY (SEQ ID NO:355); a VLCDR1 comprising the sequence QASQDINNFLN (SEQ ID NO:356); a VLCDR2 comprising the sequence DASNLET (SEQ ID NO:357); and a VLCDR3 comprising the sequence QQYGNLPFT (SEQ ID NO:358).

In one embodiment, the variable heavy chain (VH) comprising CDR1, CDR2 and CDR3 according to SEQ ID NOS: 359, 360 and 361 respectively, and a variable light chain (VL) comprising CDR1, CDR2 and CDR3 according to SEQ ID NOS: 362, 363 and 364, respectively. The methods for the generation of such scFv and the DNA encoding the respective variable regions are fully disclosed within Patent EP3436030B1. In some embodiments, the provided bispecific molecule comprises a VHCDR1 comprising the sequence GGSISSGGF (SEQ ID NO:359); a VHCDR2 comprising the sequence HHSGS (SEQ ID NO:360); a VHCDR3 comprising the sequence LVYCGGDCYSGFDY (SEQ ID NO:361); a VLCDR1 comprising the sequence QASQDINNFLN (SEQ ID NO:362; a VLCDR2 comprising the sequence DASNLET (SEQ ID NO:363); and a VLCDR3 comprising the sequence QQYGNLPFT (SEQ ID NO:364).

In some embodiments, the second binding domain and/or one or more further additional binding domains of the provide bispecific molecule specifically binds EPCAM. Bispecific molecules provided herein that specifically bind EPCAM are useful in treating an epithelial cancer. The provided bispecific molecules that specifically bind EPCAM are useful in treating diseases tumorous diseases, such as breast cancer, colon cancer, prostate cancer, head and neck cancer, skin cancer (melanoma), cancers of the genito-urinary tract (e.g., ovarian cancer, endometrial cancer, cervix cancer and kidney cancer, lung cancer, gastric cancer, cancer of the small intestine, liver cancer, pancreas cancer, gall bladder cancer, cancers of the bile duct, esophagus cancer, cancer of the salivatory glands and cancer of the thyroid gland.

In some embodiments, the second binding domain and/or one or more further additional binding domains of the provide bispecific molecule specifically binds a tissue-specific antigen from a melanoma. In some embodiments, the second binding domain and/or one or more additional binding domains specifically binds an antigen selected from: MART-1, tyrosinase, and GP100.

In some embodiments, the second binding domain and/or one or more further additional binding domains of the provide bispecific molecule specifically binds a tissue-specific antigen from a prostate cancer. In some embodiments, the second binding domain and/or one or more additional binding domains specifically binds an antigen selected from: prostatic acid phosphatase (PAP) and prostate-specific antigen (PSA).

In some embodiments, the second binding domain and/or one or more further additional binding domains of the provide bispecific molecule specifically binds a transformation-related molecule. In some embodiments, the second binding domain one or more additional binding domains specifically binds ErbB2 (HER2). In some embodiments, the second binding domain and/or another binding domain of the bispecific molecule specifically binds an onco-fetal antigen. In some embodiments, the second binding domain and/or one or more additional binding domains specifically CEA.

In some embodiments, the second binding domain and/or one or more further additional binding domains of the provide bispecific molecule specifically binds a cancer testis (CT) antigen. In some embodiments, the second binding domain and/or one or more additional binding domains specifically binds NY-ESO-1 or LAGE-1a.

In some embodiments, the second binding domain and/or one or more further additional binding domains of the provide bispecific molecule specifically binds a tumor differentiation antigen. In some embodiments, the second binding domain and/or one or more additional binding domains specifically binds MART1/MelanA, gp100 (Pmel 17), tyrosinase, TRP1, or TRP2.

In some embodiments, the second binding domain and/or one or more further additional binding domains of the provide bispecific molecule specifically binds a tumor-specific multilineage antigen. In some embodiments, the second binding domain and/or one or more additional binding domains specifically binds: MAGE1, MAGE3, BAGE, GAGE1, GAGE2, or p15.

In some embodiments, the second binding domain and/or one or more further additional binding domains of the provide bispecific molecule specifically binds an overexpressed embryonic antigen. In some embodiments, the second binding domain one or more additional binding domains specifically binds CEA.

In some embodiments, the second binding domain and/or one or more further additional binding domains of the provide bispecific molecule specifically binds specifically binds an antigen corresponding to an overexpressed oncogene or mutated tumor-suppressor gene product. In some embodiments, the second binding domain one or more additional binding domains specifically binds p53, Ras, or HER2/neu.

In some embodiments, the second binding domain and/or one or more further additional binding domains of the provide bispecific molecule specifically binds a tumor antigen resulting from a chromosomal translocation. In some embodiments, the second binding domain one or more additional binding domains specifically binds a tumor antigen selected from: BCR-ABL, E2A-PRL, H4-RET, IGH-IGK, and MYL-RAR.

In some embodiments, the second binding domain and/or one or more further additional binding domains of the provide bispecific molecule specifically binds an epitope contained on an antigen on the surface of an immune cell. In some embodiments the second binding domain and/or one or more additional binding domains specifically binds a cell surface antigen expressed on the surface of a cell associated with a disorder of the immune system. In some embodiments, the second binding domain and/or one or more additional binding domains specifically binds a cell surface antigen expressed on the surface of a cell associated with an inflammatory disorder or autoimmune disease (e.g., rheumatoid arthritis and type 1 diabetes). In some embodiments the second binding domain and/or one or more additional binding domains specifically binds a cell surface antigen selected from CXCR3, and CCR5. AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IL-6 receptor, integrin a4, integrin a407, Lama glama, LFA-1 (CD1 1a), MEDI-528, myostatin, OX-40, integrin 07, scleroscin, SOST.

In some embodiments, the second binding domain and/or one or more further additional binding domains of the provide bispecific molecule specifically binds an epitope contained on an antigen on the surface of a diseased cell.

In some embodiments, the second binding domain and/or one or more further additional binding domains of the provide bispecific molecule specifically binds an epitope contained on an antigen on the surface of an infected cell.

In some embodiments, the second binding domain and/or one or more further additional binding domains of the provide bispecific molecule specifically binds an epitope contained on an antigen on the surface of a pathogen.

In some embodiments, the second binding domain and/or one or more further additional binding domains of the provide bispecific molecule specifically binds a pathogen associated antigen. In some embodiments, the second binding domain and/or another binding domain specifically binds the Epstein Barr virus antigen EBVA. In some embodiments, the second binding domain and/or another binding domain specifically binds human papillomavirus (HPV) antigen E6 or E7.

In some embodiments, the second binding domain and/or one or more further additional binding domains of the provide bispecific molecule specifically binds a pathogen associated antigen. In some embodiments, the second binding domain and/or another binding domain specifically binds the Epstein Barr virus antigen EBVA. In some enbodirnents, the secoiid binding domain and/or another binding donai specifically an antigen selected from anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, a Pseudomonas aeruginosa surface protein, rabies virus glycoprotein, a respiratory syncytial virus coat protein and TNF-α.

### Multifunctional Bispecific Molecules

In some embodiments, the bispecific molecules provided herein have, in addition binding domains that bind a cell surface target on the surface of an immune cell and target cell for the purpose of redirecting an immune effector cell to a target cell of interest, a further function. In this format, the bispecific molecule is trifunctional or multifunctional as it provides a further function such as a fully functional Fc constant domain mediating antibody-dependent cellular cytotoxicity through recruitment of effector cells like NK cells, a label (fluorescent etc.), a therapeutic agent such as a toxin or radionuclide, and/or means to enhance serum half-life, etc.

In some embodiments, the provided bispecific molecules comprise components that extend serum half-life of the bispecific molecule, including, for example, a peptide, protein or protein domain that is fused or otherwise attached to the bispecific molecule. Such peptides, proteins and/or protein domains include for example, peptides that bind to other proteins with preferred pharmacokinetic profile in the human body such as serum albumin (see, e.g., Intl. Publ. No. WO 2009/127691), or peptides that bind neonatal Fc receptor (FcRn, see e.g., Intl. Publ. No. WO 2007/098420. In some embodiments, the provided bispecific molecules are linked (e.g. via peptide bond) with a fusion partner (such as a protein or polypeptide or peptide), e.g. for the purpose of extending the construct's serum half-life. These fusion partners can be selected from human serum albumin (“HSA” or “HALB”) as wells as sequence variants thereof, peptides binding to HSA, peptides binding to FcRn (“FcRn BP”), or constructs comprising an (antibody derived) Fc region. In some embodiments, the provided bispecific molecules comprise a constant region of an immunoglobulin (an Fc domains) or a variant thereof. Such variants of Fc domains may be optimized/modified in order to allow the desired pairing of dimers or multimers, to abolish Fc receptor binding (e.g. the Fcγ receptor) or for other reasons well known and understood in the art. In general, the fusion partners may be linked to the N-terminus or to the C-terminus of the bispecific molecule, either directly (e.g. via peptide bond) or through a peptide linker such as (GGGGS)ₙ (wherein “n” is an integer of 2 or greater, e.g. 2 or 3 or 4).

Alternatively the half-life of the provided bispecific molecule can be extended by chemical modification, by for example, pegylation.

### Switch Linkers

The provided bispecific molecules comprise one or more binding domains that comprise a VH and a VL that is covalently connected by an allosteric switch linker that contains a calmodulin polypeptide sequence, and wherein the binding domain specifically binds an antigen of interest expressed on the surface of a cell in the presence of Ca+ and a calmodulin binding ligand.

In some embodiments, the provided bispecific molecule contains a format according to a format depicted in FIG. 3, wherein the indicated scFv domain binds to an immune effector cell antigen such as CD3 as one of its targeting moieties and contains a switch linker.

In some embodiments, the first binding domain of the provided bispecific molecule comprises a linker containing a calmodulin with the complete or partial sequence homologous to a switch linker polypeptide sequence disclosed in Table 2. In some embodiments, the first binding domain comprises a linker containing a calmodulin with a partial or variant sequence homologous to a switch linker polypeptide sequence disclosed in Table 2, or a circular permutation variant of calmodulin homologous to one or part of one of the polypeptides in Table 2. In some embodiments, the bispecific molecule comprises a switch linker containing a sequence that has at least 65%, 70%, 75%, 80%, 85%, 90%, 95%, 96%, 97%, 98% or 99% identity to a linker sequence disclosed in Table 2. In some embodiments, the bispecific molecule comprises a switch linker containing an amino acid sequence that contains 1-25, 1-20, 1-15, 1-10, 1-5 or 5-20 amino acid additions, substitutions, and/or deletions compared to a switch linker sequence disclosed in Table 2.

In some embodiments, the first binding domain of the provided bispecific molecule comprises a linker containing a calmodulin with the complete sequence disclosed in SEQ ID NO:92. In some embodiments, the first binding domain comprises a linker containing a calmodulin partial sequence that is homologous to a polypeptide sequence disclosed in SEQ ID NO:92. In some embodiments, the first binding domain comprises a linker containing a calmodulin with a polypeptide sequence disclosed in any one of SEQ ID NOS:92-287.

In some embodiments, the first binding domain of the provided bispecific molecule comprises a linker containing a calmodulin partial sequence that is homologous to a polypeptide sequence disclosed in any one of SEQ ID NOS:92-287. In some embodiments, the first binding domain comprises a linker containing a sequence that has at least 65%, 70%, 75%, 80%, 85%, 90%, 95%, 96%, 97%, 98% or 99% identity to the linker sequence disclosed in any one of SEQ ID NOS:92-287 the first binding domain comprises a linker containing a sequence that contains 1-25, 1-20, 1-15, 1-10, 1-5 or 5-20 amino acid additions, substitutions, and/or deletions compared to the switch linker sequence disclosed in in any one of SEQ ID NOS:92-287.

TABLE 2
Exemplary linker sequences contained in a switch linker

| SEQ | |
|---|---|
| ID: | Sequence |
| 92 | ADQLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQVNY |
| | EEFVQMMTAK |
| 93 | ADQLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQVNY |
| | EEFVQMMTAK |
| 94 | DQLTEEQIAEFKEAFSLFDKDGDGTITTKELGTVMRSLGQNPTEAELQDMINEVDADGNGTIDFPEFLTM |
| | MARKMKDTDSEEEIREAFRVFDKDGNGYISAAELRHVMTNLGEKLTDEEVDEMIREADIDGDGQVNYE |
| | EFVQMMTAK |
| 95 | MKDTDSEEEIREAFRVFDKDGNGYISAAELRHVMTNLGEKLTDEEVDEMIREADIDGDGQVNYEEFVQ |
| | MMTAKGGSGADQLTEEQIAEFKEAFSLFDKDGDGTITTKELGTVMRSLGQNPTEAELQDMINEVDADG |
| | NGTIDFPEFLTMMARK |
| 96 | SEEEIREAFRVFDKDGNGYISAAELRHVMTNLGEKLTDEEVDEMIREADIDGDGQVNYEEFVQMMTAK |
| | GGSGADQLTEEQIAEFKEAFSLFDKDGDGTITTKELGTVMRSLGQNPTEAELQDMINEVDADGNGTIDF |
| | PEFLTMMARKMKDTD |
| 97 | KGGSGADQLTEEQIAEFKEAFSLFDKDGDGTITTKELGTVMRSLGQNPTEAELQDMINEVDADGNGTID |
| | FPEFLTMMARKMKDTDSEEEIREAFRVFDKDGNGYISAAELRHVMTNLGEKLTDEEVDEMIREADIDGD |
| | GQVNYEEFVQMMTA |
| 98 | AKGGSGADQLTEEQIAEFKEAFSLFDKDGDGTITTKELGTVMRSLGQNPTEAELQDMINEVDADGNGTI |
| | DFPEFLTMMARKMKDTDSEEEIREAFRVFDKDGNGYISAAELRHVMTNLGEKLTDEEVDEMIREADIDG |
| | DGQVNYEEFVQMMT |
| 99 | LTEEQIAEFKEAFSLFDKDGDGTITTKELGTVMRSLGQNPTEAELQDMINEVDADGNGTIDFPEFLTM |
| | MARKMKDTDSEEEIREAFRVFDKDGNGYISAAELRHVMTNLGEKLTDEEVDEMIREADIDGDGQVNY |
| | EEFVQMMTAK |
| 100 | EQIAEFKEAFSLFDKDGDGTITTKELGTVMRSLGQNPTEAELQDMINEVDADGNGTIDFPEFLTMMA |
| | RKMKDTDSEEEIREAFRVFDKDGNGYISAAELRHVMTNLGEKLTDEEVDEMIREADIDGDGQVNYEEF |
| | VQMMTAK |
| 101 | AEFKEAFSLFDKDGDGTITTKELGTVMRSLGQNPTEAELQDMINEVDADGNGTIDFPEFLTMMARK |
| | MKDTDSEEEIREAFRVFDKDGNGYISAAELRHVMTNLGEKLTDEEVDEMIREADIDGDGQVNYEEFV |
| | QMMTAK |
| 102 | AFSLFDKDGDGTITTKELGTVMRSLGQNPTEAELQDMINEVDADGNGTIDFPEFLTMMARKMKDTD |
| | SEEEIREAFRVFDKDGNGYISAAELRHVMTNLGEKLTDEEVDEMIREADIDGDGQVNYEEFVQMMTA |
| | K |
| 103 | DKDGDGTITTKELGTVMRSLGQNPTEAELQDMINEVDADGNGTIDFPEFLTMMARKMKDTDSEEEI |
| | REAFRVFDKDGNGYISAAELRHVMTNLGEKLTDEEVDEMIREADIDGDGQVNYEEFVQMMTAK |
| 104 | GTITTKELGTVMRSLGQNPTEAELQDMINEVDADGNGTIDFPEFLTMMARKMKDTDSEEEIREAFRV |
| | FDKDGNGYISAAELRHVMTNLGEKLTDEEVDEMIREADIDGDGQVNYEEFVQMMTAK |
| 105 | KELGTVMRSLGQNPTEAELQDMINEVDADGNGTIDFPEFLTMMARKMKDTDSEEEIREAFRVFDKD |
| | GNGYISAAELRHVMTNLGEKLTDEEVDEMIREADIDGDGQVNYEEFVQMMTAK |
| 106 | GQNPTEAELQDMINEVDADGNGTIDFPEFLTMMARKMKDTDSEEEIREAFRVFDKDGNGYISAAELR |
| | HVMTNLGEKLTDEEVDEMIREADIDGDGQVNYEEFVQMMTAK |
| 107 | ADQLTEEQIAEFKEAFSLFDKDGDGTITTKELGTVMRSLGQNPTEAELQDMINEVDADGNGTIDFPEF |
| | LTMMARKMKDTDSEEEIREAFRVFDKDGNGYISAAELRHVMTNLGEKLTDEEVDEMIREADIDGDG |
| | QVNYEEFVQ |
| 108 | LTEEQIAEFKEAFSLFDKDGDGTITTKELGTVMRSLGQNPTEAELQDMINEVDADGNGTIDFPEFLTM |
| | MARKMKDTDSEEEIREAFRVFDKDGNGYISAAELRHVMTNLGEKLTDEEVDEMIREADIDGDGQVNY |
| | EEFVQ |
| 109 | EQIAEFKEAFSLFDKDGDGTITTKELGTVMRSLGQNPTEAELQDMINEVDADGNGTIDFPEFLTMMA |
| | RKMKDTDSEEEIREAFRVFDKDGNGYISAAELRHVMTNLGEKLTDEEVDEMIREADIDGDGQVNYEEF |
| | VQ |
| 110 | AEFKEAFSLFDKDGDGTITTKELGTVMRSLGQNPTEAELQDMINEVDADGNGTIDFPEFLTMMARK |
| | MKDTDSEEEIREAFRVFDKDGNGYISAAELRHVMTNLGEKLTDEEVDEMIREADIDGDGQVNYEEFV |
| | Q |
| 111 | AFSLFDKDGDGTITTKELGTVMRSLGQNPTEAELQDMINEVDADGNGTIDFPEFLTMMARKMKDTD |
| | SEEEIREAFRVFDKDGNGYISAAELRHVMTNLGEKLTDEEVDEMIREADIDGDGQVNYEEFVQ |
| 112 | DKDGDGTITTKELGTVMRSLGQNPTEAELQDMINEVDADGNGTIDFPEFLTMMARKMKDTDSEEEI |
| | REAFRVFDKDGNGYISAAELRHVMTNLGEKLTDEEVDEMIREADIDGDGQVNYEEFVQ |
| 113 | GTITTKELGTVMRSLGQNPTEAELQDMINEVDADGNGTIDFPEFLTMMARKMKDTDSEEEIREAFRV |
| | FDKDGNGYISAAELRHVMTNLGEKLTDEEVDEMIREADIDGDGQVNYEEFVQ |
| 114 | KELGTVMRSLGQNPTEAELQDMINEVDADGNGTIDFPEFLTMMARKMKDTDSEEEIREAFRVFDKD |
| | GNGYISAAELRHVMTNLGEKLTDEEVDEMIREADIDGDGQVNYEEFVQ |
| 115 | GQNPTEAELQDMINEVDADGNGTIDFPEFLTMMARKMKDTDSEEEIREAFRVFDKDGNGYISAAELR |
| | HVMTNLGEKLTDEEVDEMIREADIDGDGQVNYEEFVQ |
| 116 | ADQLTEEQIAEFKEAFSLFDKDGDGTITTKELGTVMRSLGQNPTEAELQDMINEVDADGNGTIDFPEF |
| | LTMMARKMKDTDSEEEIREAFRVFDKDGNGYISAAELRHVMTNLGEKLTDEEVDEMIREADIDGDG |
| | QVNYEEFVK |
| 117 | LTEEQIAEFKEAFSLFDKDGDGTITTKELGTVMRSLGQNPTEAELQDMINEVDADGNGTIDFPEFLTM |
| | MARKMKDTDSEEEIREAFRVFDKDGNGYISAAELRHVMTNLGEKLTDEEVDEMIREADIDGDGQVNY |
| | EEFVK |
| 118 | EQIAEFKEAFSLFDKDGDGTITTKELGTVMRSLGQNPTEAELQDMINEVDADGNGTIDFPEFLTMMA |
| | RKMKDTDSEEEIREAFRVFDKDGNGYISAAELRHVMTNLGEKLTDEEVDEMIREADIDGDGQVNYEEF |
| | VK |
| 119 | AEFKEAFSLFDKDGDGTITTKELGTVMRSLGQNPTEAELQDMINEVDADGNGTIDFPEFLTMMARK |
| | MKDTDSEEEIREAFRVFDKDGNGYISAAELRHVMTNLGEKLTDEEVDEMIREADIDGDGQVNYEEFVK |
| 120 | AFSLFDKDGDGTITTKELGTVMRSLGQNPTEAELQDMINEVDADGNGTIDFPEFLTMMARKMKDTD |
| | SEEEIREAFRVFDKDGNGYISAAELRHVMTNLGEKLTDEEVDEMIREADIDGDGQVNYEEFVK |
| 121 | DKDGDGTITTKELGTVMRSLGQNPTEAELQDMINEVDADGNGTIDFPEFLTMMARKMKDTDSEEEI |
| | REAFRVFDKDGNGYISAAELRHVMTNLGEKLTDEEVDEMIREADIDGDGQVNYEEFVK |
| 122 | GTITTKELGTVMRSLGQNPTEAELQDMINEVDADGNGTIDFPEFLTMMARKMKDTDSEEEIREAFRV |
| | FDKDGNGYISAAELRHVMTNLGEKLTDEEVDEMIREADIDGDGQVNYEEFVK |
| 123 | KELGTVMRSLGQNPTEAELQDMINEVDADGNGTIDFPEFLTMMARKMKDTDSEEEIREAFRVFDKD |
| | GNGYISAAELRHVMTNLGEKLTDEEVDEMIREADIDGDGQVNYEEFVK |
| 124 | GQNPTEAELQDMINEVDADGNGTIDFPEFLTMMARKMKDTDSEEEIREAFRVFDKDGNGYISAAELR |
| | HVMTNLGEKLTDEEVDEMIREADIDGDGQVNYEEFVK |
| 125 | ADQLTEEQIAEFKEAFSLFDKDGDGTITTKELGTVMRSLGQNPTEAELQDMINEVDADGNGTIDFPEF |
| | LTMMARKMKDTDSEEEIREAFRVFDKDGNGYISAAELRHVMTNLGEKLTDEEVDEMIREADIDGDG |
| | QVNY |
| 126 | LTEEQIAEFKEAFSLFDKDGDGTITTKELGTVMRSLGQNPTEAELQDMINEVDADGNGTIDFPEFLTM |
| | MARKMKDTDSEEEIREAFRVFDKDGNGYISAAELRHVMTNLGEKLTDEEVDEMIREADIDGDGQVNY |
| 127 | EQIAEFKEAFSLFDKDGDGTITTKELGTVMRSLGQNPTEAELQDMINEVDADGNGTIDFPEFLTMMA |
| | RKMKDTDSEEEIREAFRVFDKDGNGYISAAELRHVMTNLGEKLTDEEVDEMIREADIDGDGQVNY |
| 128 | AEFKEAFSLFDKDGDGTITTKELGTVMRSLGQNPTEAELQDMINEVDADGNGTIDFPEFLTMMARK |
| | MKDTDSEEEIREAFRVFDKDGNGYISAAELRHVMTNLGEKLTDEEVDEMIREADIDGDGQVNY |
| 129 | AFSLFDKDGDGTITTKELGTVMRSLGQNPTEAELQDMINEVDADGNGTIDFPEFLTMMARKMKDTD |
| | SEEEIREAFRVFDKDGNGYISAAELRHVMTNLGEKLTDEEVDEMIREADIDGDGQVNY |
| 130 | DKDGDGTITTKELGTVMRSLGQNPTEAELQDMINEVDADGNGTIDFPEFLTMMARKMKDTDSEEEI |
| | REAFRVFDKDGNGYISAAELRHVMTNLGEKLTDEEVDEMIREADIDGDGQVNY |
| 131 | GTITTKELGTVMRSLGQNPTEAELQDMINEVDADGNGTIDFPEFLTMMARKMKDTDSEEEIREAFRV |
| | FDKDGNGYISAAELRHVMTNLGEKLTDEEVDEMIREADIDGDGQVNY |
| 132 | KELGTVMRSLGQNPTEAELQDMINEVDADGNGTIDFPEFLTMMARKMKDTDSEEEIREAFRVFDKD |
| | GNGYISAAELRHVMTNLGEKLTDEEVDEMIREADIDGDGQVNY |
| 133 | GQNPTEAELQDMINEVDADGNGTIDFPEFLTMMARKMKDTDSEEEIREAFRVFDKDGNGYISAAELR |
| | HVMTNLGEKLTDEEVDEMIREADIDGDGQVNY |
| 135 | EIREAFRVFDKDGNGYISAAELRHVMTNLGEKLTDEEVDEMIREADIDGDGQVNYEEFVQMMTAKG |
| | GSGADQLTEEQIAEFKEAFSLFDKDGDGTITTKELGTVMRSLGQNPTEAELQDMINEVDADGNGTIDF |
| | PEFLTMMARKMKDTD |
| 136 | VFDKDGNGYISAAELRHVMTNLGEKLTDEEVDEMIREADIDGDGQVNYEEFVQMMTAKGGSGADQ |
| | LTEEQIAEFKEAFSLFDKDGDGTITTKELGTVMRSLGQNPTEAELQDMINEVDADGNGTIDFPEFLTM |
| | MARKMKDTD |
| 137 | GNGYISAAELRHVMTNLGEKLTDEEVDEMIREADIDGDGQVNYEEFVQMMTAKGGSGADQLTEEQI |
| | AEFKEAFSLFDKDGDGTITTKELGTVMRSLGQNPTEAELQDMINEVDADGNGTIDFPEFLTMMARK |
| | MKDTD |
| 138 | SAAELRHVMTNLGEKLTDEEVDEMIREADIDGDGQVNYEEFVQMMTAKGGSGADQLTEEQIAEFKE |
| | AFSLFDKDGDGTITTKELGTVMRSLGQNPTEAELQDMINEVDADGNGTIDFPEFLTMMARKMKDTD |
| 139 | RHVMTNLGEKLTDEEVDEMIREADIDGDGQVNYEEFVQMMTAKGGSGADQLTEEQIAEFKEAFSLF |
| | DKDGDGTITTKELGTVMRSLGQNPTEAELQDMINEVDADGNGTIDFPEFLTMMARKMKDTD |
| 140 | NLGEKLTDEEVDEMIREADIDGDGQVNYEEFVQMMTAKGGSGADQLTEEQIAEFKEAFSLFDKDGD |
| | GTITTKELGTVMRSLGQNPTEAELQDMINEVDADGNGTIDFPEFLTMMARKMKDTD |
| 141 | VDEMIREADIDGDGQVNYEEFVQMMTAKGGSGADQLTEEQIAEFKEAFSLFDKDGDGTITTKELGTV |
| | MRSLGQNPTEAELQDMINEVDADGNGTIDFPEFLTMMARKMKDTD |
| 142 | SEEEIREAFRVFDKDGNGYISAAELRHVMTNLGEKLTDEEVDEMIREADIDGDGQVNYEEFVQMMTA |
| | KGGSGADQLTEEQIAEFKEAFSLFDKDGDGTITTKELGTVMRSLGQNPTEAELQDMINEVDADGNGTI |
| | DFPEFLTMMARK |
| 143 | EIREAFRVFDKDGNGYISAAELRHVMTNLGEKLTDEEVDEMIREADIDGDGQVNYEEFVQMMTAKG |
| | GSGADQLTEEQIAEFKEAFSLFDKDGDGTITTKELGTVMRSLGQNPTEAELQDMINEVDADGNGTIDF |
| | PEFLTMMARK |
| 144 | VFDKDGNGYISAAELRHVMTNLGEKLTDEEVDEMIREADIDGDGQVNYEEFVQMMTAKGGSGADQ |
| | LTEEQIAEFKEAFSLFDKDGDGTITTKELGTVMRSLGQNPTEAELQDMINEVDADGNGTIDFPEFLTM |
| | MARK |
| 145 | GNGYISAAELRHVMTNLGEKLTDEEVDEMIREADIDGDGQVNYEEFVQMMTAKGGSGADQLTEEQI |
| | AEFKEAFSLFDKDGDGTITTKELGTVMRSLGQNPTEAELQDMINEVDADGNGTIDFPEFLTMMARK |
| 146 | SAAELRHVMTNLGEKLTDEEVDEMIREADIDGDGQVNYEEFVQMMTAKGGSGADQLTEEQIAEFKE |
| | AFSLFDKDGDGTITTKELGTVMRSLGQNPTEAELQDMINEVDADGNGTIDFPEFLTMMARK |
| 147 | RHVMTNLGEKLTDEEVDEMIREADIDGDGQVNYEEFVQMMTAKGGSGADQLTEEQIAEFKEAFSLF |
| | DKDGDGTITTKELGTVMRSLGQNPTEAELQDMINEVDADGNGTIDFPEFLTMMARK |
| 148 | NLGEKLTDEEVDEMIREADIDGDGQVNYEEFVQMMTAKGGSGADQLTEEQIAEFKEAFSLFDKDGD |
| | GTITTKELGTVMRSLGQNPTEAELQDMINEVDADGNGTIDFPEFLTMMARK |
| 149 | VDEMIREADIDGDGQVNYEEFVQMMTAKGGSGADQLTEEQIAEFKEAFSLFDKDGDGTITTKELGTV |
| | MRSLGQNPTEAELQDMINEVDADGNGTIDFPEFLTMMARK |
| 150 | SEEEIREAFRVFDKDGNGYISAAELRHVMTNLGEKLTDEEVDEMIREADIDGDGQVNYEEFVQMMTA |
| | KGGSGADQLTEEQIAEFKEAFSLFDKDGDGTITTKELGTVMRSLGQNPTEAELQDMINEVDADGNGTI |
| | DFPEFLTMMARD |
| 151 | SEEEIREAFRVFDKDGNGYISAAELRHVMTNLGEKLTDEEVDEMIREADIDGDGQVNYEEFVQMMTA |
| | KGGSGADQLTEEQIAEFKEAFSLFDKDGDGTITTKELGTVMRSLGQNPTEAELQDMINEVDADGNGTI |
| | DFPEFLT |
| 152 | EIREAFRVFDKDGNGYISAAELRHVMTNLGEKLTDEEVDEMIREADIDGDGQVNYEEFVQMMTAKG |
| | GSGADQLTEEQIAEFKEAFSLFDKDGDGTITTKELGTVMRSLGQNPTEAELQDMINEVDADGNGTIDF |
| | PEFLT |
| 153 | VFDKDGNGYISAAELRHVMTNLGEKLTDEEVDEMIREADIDGDGQVNYEEFVQMMTAKGGSGADQ |
| | LTEEQIAEFKEAFSLFDKDGDGTITTKELGTVMRSLGQNPTEAELQDMINEVDADGNGTIDFPEFLT |
| 154 | GNGYISAAELRHVMTNLGEKLTDEEVDEMIREADIDGDGQVNYEEFVQMMTAKGGSGADQLTEEQI |
| | AEFKEAFSLFDKDGDGTITTKELGTVMRSLGQNPTEAELQDMINEVDADGNGTIDFPEFLT |
| 155 | SAAELRHVMTNLGEKLTDEEVDEMIREADIDGDGQVNYEEFVQMMTAKGGSGADQLTEEQIAEFKE |
| | AFSLFDKDGDGTITTKELGTVMRSLGQNPTEAELQDMINEVDADGNGTIDFPEFLT |
| 156 | RHVMTNLGEKLTDEEVDEMIREADIDGDGQVNYEEFVQMMTAKGGSGADQLTEEQIAEFKEAFSLF |
| | DKDGDGTITTKELGTVMRSLGQNPTEAELQDMINEVDADGNGTIDFPEFLT |
| 157 | NLGEKLTDEEVDEMIREADIDGDGQVNYEEFVQMMTAKGGSGADQLTEEQIAEFKEAFSLFDKDGD |
| | GTITTKELGTVMRSLGQNPTEAELQDMINEVDADGNGTIDFPEFLT |
| 158 | VDEMIREADIDGDGQVNYEEFVQMMTAKGGSGADQLTEEQIAEFKEAFSLFDKDGDGTITTKELGTV |
| | MRSLGQNPTEAELQDMINEVDADGNGTIDFPEFLT |
| 159 | ADQLTEEQIAEFKEAFSLFDKDGDGTITTKELGTVMRSLGQNPTEAELQDMINEVDADGNGTIDFPEF |
| | LTMMARKMKDTDSEEEIREAFRVFDKDGNGYISAAELRHVMTNLGEKLTDEEVDEMIREADIDGDG |
| | QVNYEEFVQMMTAKGGSG |
| 160 | ADQLTEEQIAEFKEAFSLFDKDGDGTITTKELGTVMRSLGQNPTEAELQDMINEVDADGNGTIDFPEF |
| | LTMMARKMKDTDSEEEIREAFRVFDKDGNGYISAAELRHVMTNLGEKLTDEEVDEMIREADIDGDG |
| | QVNYEEFVQMMTAKG |
| 161 | ADQLTEEQIAEFKEAFSLFDKDGDGTITTKELGTVMRSLGQNPTEAELQDMINEVDADGNGTIDFPEF |
| | LTMMARKMKDTDSEEEIREAFRVFDKDGNGYISAAELRHVMTNLGEKLTDEEVDEMIREADIDGDG |
| | QVNYEEFVQMM |
| 162 | EEQIAEFKEAFSLFDKDGDGTITTKELGTVMRSLGQNPTEAELQDMINEVDADGNGTIDFPEFLTMM |
| | ARKMKDTDSEEEIREAFRVFDKDGNGYISAAELRHVMTNLGEKLTDEEVDEMIREADIDGDGQVNYE |
| | EFVQMMTAKGGSG |
| 163 | DKDGDGTITTKELGTVMRSLGQNPTEAELQDMINEVDADGNGTIDFPEFLTMMARKMKDTDSEEEI |
| | REAFRVFDKDGNGYISAAELRHVMTNLGEKLT |
| 164 | GQNPTEAELQDMINEVDADGNGTIDFPEFLTMMARKMKDTDSEEEIREAFRVFDKDGNGYISAAELR |
| | HVMTNLGEKLTDEEVDEMIREA |
| 165 | GQNPTEAELQDMINEVDADGNGTIDFPEFLTMMARKMKDTDSEEEIREAFRVFDKDGNGYISAAELR |
| | HVMTNLGEKLTDEEVDEMIREADIDGDGQVNYEEFVQMMTAKGGSG |
| 166 | NGTIDFPEFLTMMARKMKDTDSEEEIREAFRVFDKDGNGYISAAELRHVMTNLGEKLTDEEVDEMIRE |
| | A |
| 167 | NGTIDFPEFLTMMARKMKDTDSEEEIREAFRVFDKDGNGYISAAELRHVMTNLGEKLTDEEVDEMIRE |
| | ADIDGDGQVNYEEFVQMMTAKGGSG |
| 168 | DKDGDGYISAAELRHVMTNLGEKLTDEEVDEMIREA |
| 169 | MTNLGEKLTDEEVDEMIREA |
| 170 | MTNLGEKLTDEEVDEMIREADIDGDGQVNYEEFVQMMTAK |
| 171 | DGDGTITTKELGTVMRSLGQNPTEAELQDMINEVDADGNGTIDFPEFLTMMARKMKDTDSEEEIREA |
| | FRVFDKDGNGYISAAELRHVMTNLGEKLTDEEVDEMIREA |
| 172 | NGTIDFPEFLTMMARKMKDTDSEEEIREAFRVFDKDGNGYISAAELRHVMTNLGEKLT |
| 173 | DKDGEKLT |
| 174 | DKDGDGQVNYEEFVQMMTAK |
| 177 | ADQLTEEQIAEFKEAFSLFDKDGDGTITTKELGTVMRSLGQNPTEAELQDMINEVDADGNGTIDFPEF |
| | LTMMARKMKDTD |
| 178 | EFKEAFSLFDKDGDGTITTKELGTVMRSLGQNPTEAELQDMINEVDADGNGTIDFPEFLTMMARKMK |
| | DTD |
| 179 | NGTIDFPEFLTMMARKMKDTD |
| 180 | MTNLGEKLTDEEVDEMIREADIDGDGQVNYEEFVQMMTAKGGSGADQLTEEQIAEFKEAFSLFDKD |
| | GDGTITTKELGTVMRSLGQNPTEAELQDMINEVDADGNGTIDFPEFLTMMARKMKDTD |
| 181 | SEEEIREAFRVFDKDGNGYISAAELRHVMTNLGEKLTDEEVDEMIREADIDGDGQVNYEEFVQMMTA |
| | KGGSGADQLTEEQIAEFKEAFSLFDKDGDGTITTKELGTVMRSLGQNPTEAELQDMINEVDADGNGTI |
| | DFPEFLTMMARKMK |
| 182 | EIREAFRVFDKDGNGYISAAELRHVMTNLGEKLTDEEVDEMIREADIDGDGQVNYEEFVQMMTAKG |
| | GSGADQLTEEQIAEFKEAFSLFDKDGDGTITTKELGTVMRSLGQNPTEAELQDMINEVDADGNGTIDF |
| | PEFLTMMARKMKDTD |
| 183 | ADQLTEEQIAEFKEAFSLFDKDGDGTITTKELGTVMRSLGQNPTEAELQDMINEVDADGNGTIDFPEF |
| | LTMMARKMKDTDSEEEIREAFRVFDKDGNGYISAAELRHVMTNLGEKLTDEEVDEMIREADIDGDG |
| | QVNYEEFVQMMTAKSG |
| 184 | DQLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQV |
| | NYEEFVQMMTAKSG |
| 185 | QLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQV |
| | NYEEFVQMMTAKSG |
| 186 | LTEEQIAEFKEAFSLFDKDGDGTITTKELGTVMRSLGQNPTEAELQDMINEVDADGNGTIDFPEFLTM |
| | MARKMKDTDSEEEIREAFRVFDKDGNGYISAAELRHVMTNLGEKLTDEEVDEMIREADIDGDGQVNY |
| | EEFVQMMTAKSG |
| 187 | TEEQIAEFKEAFSLFDKDGDGTITTKELGTVMRSLGQNPTEAELQDMINEVDADGNGTIDFPEFLTMM |
| | ARKMKDTDSEEEIREAFRVFDKDGNGYISAAELRHVMTNLGEKLTDEEVDEMIREADIDGDGQVNYE |
| | EFVQMMTAKSG |
| 188 | EEQIAEFKEAFSLFDKDGDGTITTKELGTVMRSLGQNPTEAELQDMINEVDADGNGTIDFPEFLTMM |
| | ARKMKDTDSEEEIREAFRVFDKDGNGYISAAELRHVMTNLGEKLTDEEVDEMIREADIDGDGQVNYE |
| | EFVQMMTAKSG |
| 189 | EQIAEFKEAFSLFDKDGDGTITTKELGTVMRSLGQNPTEAELQDMINEVDADGNGTIDFPEFLTMMA |
| | RKMKDTDSEEEIREAFRVFDKDGNGYISAAELRHVMTNLGEKLTDEEVDEMIREADIDGDGQVNYEEF |
| | VQMMTAKSG |
| 190 | QIAEFKEAFSLFDKDGDGTITTKELGTVMRSLGQNPTEAELQDMINEVDADGNGTIDFPEFLTMMAR |
| | KMKDTDSEEEIREAFRVFDKDGNGYISAAELRHVMTNLGEKLTDEEVDEMIREADIDGDGQVNYEEFV |
| | QMMTAKSG |
| 191 | AEFKEAFSLFDKDGDGTITTKELGTVMRSLGQNPTEAELQDMINEVDADGNGTIDFPEFLTMMARK |
| | MKDTDSEEEIREAFRVFDKDGNGYISAAELRHVMTNLGEKLTDEEVDEMIREADIDGDGQVNYEEFV |
| | QMMTAKSG |
| 192 | ADQLTEEQIAEFKEAFSLFDKDGDGTITTKELGTVMRSLGQNPTEAELQDMINEVDADGNGTIDFPEF |
| | LTMMARKMKDTDSEEEIREAFRVFDKDGNGYISAAELRHVMTNLGEKLTDEEVDEMIREADIDGDG |
| | QVNYEEFVQMMTASG |
| 193 | DQLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQV |
| | NYEEFVQMMTASG |
| 194 | QLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQV |
| | NYEEFVQMMTASG |
| 195 | LTEEQIAEFKEAFSLFDKDGDGTITTKELGTVMRSLGQNPTEAELQDMINEVDADGNGTIDFPEFLTM |
| | MARKMKDTDSEEEIREAFRVFDKDGNGYISAAELRHVMTNLGEKLTDEEVDEMIREADIDGDGQVNY |
| | EEFVQMMTASG |
| 196 | TEEQIAEFKEAFSLFDKDGDGTITTKELGTVMRSLGQNPTEAELQDMINEVDADGNGTIDFPEFLTMM |
| | ARKMKDTDSEEEIREAFRVFDKDGNGYISAAELRHVMTNLGEKLTDEEVDEMIREADIDGDGQVNYE |
| | EFVQMMTASG |
| 197 | EEQIAEFKEAFSLFDKDGDGTITTKELGTVMRSLGQNPTEAELQDMINEVDADGNGTIDFPEFLTMM |
| | ARKMKDTDSEEEIREAFRVFDKDGNGYISAAELRHVMTNLGEKLTDEEVDEMIREADIDGDGQVNYE |
| | EFVQMMTASG |
| 198 | EQIAEFKEAFSLFDKDGDGTITTKELGTVMRSLGQNPTEAELQDMINEVDADGNGTIDFPEFLTMMA |
| | RKMKDTDSEEEIREAFRVFDKDGNGYISAAELRHVMTNLGEKLTDEEVDEMIREADIDGDGQVNYEEF |
| | VQMMTASG |
| 199 | QIAEFKEAFSLFDKDGDGTITTKELGTVMRSLGQNPTEAELQDMINEVDADGNGTIDFPEFLTMMAR |
| | KMKDTDSEEEIREAFRVFDKDGNGYISAAELRHVMTNLGEKLTDEEVDEMIREADIDGDGQVNYEEFV |
| | QMMTASG |
| 200 | AEFKEAFSLFDKDGDGTITTKELGTVMRSLGQNPTEAELQDMINEVDADGNGTIDFPEFLTMMARK |
| | MKDTDSEEEIREAFRVFDKDGNGYISAAELRHVMTNLGEKLTDEEVDEMIREADIDGDGQVNYEEFV |
| | QMMTASG |
| 201 | ADQLTEEQIAEFKEAFSLFDKDGDGTITTKELGTVMRSLGQNPTEAELQDMINEVDADGNGTIDFPEF |
| | LTMMARKMKDTDSEEEIREAFRVFDKDGNGYISAAELRHVMTNLGEKLTDEEVDEMIREADIDGDG |
| | QVNYEEFVQMMTSG |
| 202 | DQLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQV |
| | NYEEFVQMMTSG |
| 203 | QLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQV |
| | NYEEFVQMMTSG |
| 204 | LTEEQIAEFKEAFSLFDKDGDGTITTKELGTVMRSLGQNPTEAELQDMINEVDADGNGTIDFPEFLTM |
| | MARKMKDTDSEEEIREAFRVFDKDGNGYISAAELRHVMTNLGEKLTDEEVDEMIREADIDGDGQVNY |
| | EEFVQMMTSG |
| 205 | TEEQIAEFKEAFSLFDKDGDGTITTKELGTVMRSLGQNPTEAELQDMINEVDADGNGTIDFPEFLTMM |
| | ARKMKDTDSEEEIREAFRVFDKDGNGYISAAELRHVMTNLGEKLTDEEVDEMIREADIDGDGQVNYE |
| | EFVQMMTSG |
| 206 | EEQIAEFKEAFSLFDKDGDGTITTKELGTVMRSLGQNPTEAELQDMINEVDADGNGTIDFPEFLTMM |
| | ARKMKDTDSEEEIREAFRVFDKDGNGYISAAELRHVMTNLGEKLTDEEVDEMIREADIDGDGQVNYE |
| | EFVQMMTSG |
| 207 | EQIAEFKEAFSLFDKDGDGTITTKELGTVMRSLGQNPTEAELQDMINEVDADGNGTIDFPEFLTMMA |
| | RKMKDTDSEEEIREAFRVFDKDGNGYISAAELRHVMTNLGEKLTDEEVDEMIREADIDGDGQVNYEEF |
| | VQMMTSG |
| 208 | QIAEFKEAFSLFDKDGDGTITTKELGTVMRSLGQNPTEAELQDMINEVDADGNGTIDFPEFLTMMAR |
| | KMKDTDSEEEIREAFRVFDKDGNGYISAAELRHVMTNLGEKLTDEEVDEMIREADIDGDGQVNYEEFV |
| | QMMTSG |
| 209 | AEFKEAFSLFDKDGDGTITTKELGTVMRSLGQNPTEAELQDMINEVDADGNGTIDFPEFLTMMARK |
| | MKDTDSEEEIREAFRVFDKDGNGYISAAELRHVMTNLGEKLTDEEVDEMIREADIDGDGQVNYEEFV |
| | QMMTSG |
| 210 | ADQLTEEQIAEFKEAFSLFDKDGDGTITTKELGTVMRSLGQNPTEAELQDMINEVDADGNGTIDFPEF |
| | LTMMARKMKDTDSEEEIREAFRVFDKDGNGYISAAELRHVMTNLGEKLTDEEVDEMIREADIDGDG |
| | QVNYEEFVQMMSG |
| 211 | DQLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQV |
| | NYEEFVQMMSG |
| 212 | QLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQV |
| | NYEEFVQMMSG |
| 213 | LTEEQIAEFKEAFSLFDKDGDGTITTKELGTVMRSLGQNPTEAELQDMINEVDADGNGTIDFPEFLTM |
| | MARKMKDTDSEEEIREAFRVFDKDGNGYISAAELRHVMTNLGEKLTDEEVDEMIREADIDGDGQVNY |
| | EEFVQMMSG |
| 214 | TEEQIAEFKEAFSLFDKDGDGTITTKELGTVMRSLGQNPTEAELQDMINEVDADGNGTIDFPEFLTMM |
| | ARKMKDTDSEEEIREAFRVFDKDGNGYISAAELRHVMTNLGEKLTDEEVDEMIREADIDGDGQVNYE |
| | EFVQMMSG |
| 215 | EEQIAEFKEAFSLFDKDGDGTITTKELGTVMRSLGQNPTEAELQDMINEVDADGNGTIDFPEFLTMM |
| | ARKMKDTDSEEEIREAFRVFDKDGNGYISAAELRHVMTNLGEKLTDEEVDEMIREADIDGDGQVNYE |
| | EFVQMMSG |
| 216 | EQIAEFKEAFSLFDKDGDGTITTKELGTVMRSLGQNPTEAELQDMINEVDADGNGTIDFPEFLTMMA |
| | RKMKDTDSEEEIREAFRVFDKDGNGYISAAELRHVMTNLGEKLTDEEVDEMIREADIDGDGQVNYEEF |
| | VQMMSG |
| 217 | QIAEFKEAFSLFDKDGDGTITTKELGTVMRSLGQNPTEAELQDMINEVDADGNGTIDFPEFLTMMAR |
| | KMKDTDSEEEIREAFRVFDKDGNGYISAAELRHVMTNLGEKLTDEEVDEMIREADIDGDGQVNYEEFV |
| | QMMSG |
| 218 | AEFKEAFSLFDKDGDGTITTKELGTVMRSLGQNPTEAELQDMINEVDADGNGTIDFPEFLTMMARK |
| | MKDTDSEEEIREAFRVFDKDGNGYISAAELRHVMTNLGEKLTDEEVDEMIREADIDGDGQVNYEEFV |
| | QMMSG |
| 219 | ADQLTEEQIAEFKEAFSLFDKDGDGTITTKELGTVMRSLGQNPTEAELQDMINEVDADGNGTIDFPEF |
| | LTMMARKMKDTDSEEEIREAFRVFDKDGNGYISAAELRHVMTNLGEKLTDEEVDEMIREADIDGDG |
| | QVNYEEFVQMSG |
| 220 | DQLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQV |
| | NYEEFVQMSG |
| 221 | QLTEEQIAEFKEAFSLFDKDGDGTITTKELGTVMRSLGQNPTEAELQDMINEVDADGNGTIDFPEFLT |
| | MMARKMKDTDSEEEIREAFRVFDKDGNGYISAAELRHVMTNLGEKLTDEEVDEMIREADIDGDGQV |
| | NYEEFVQMSG |
| 222 | LTEEQIAEFKEAFSLFDKDGDGTITTKELGTVMRSLGQNPTEAELQDMINEVDADGNGTIDFPEFLTM |
| | MARKMKDTDSEEEIREAFRVFDKDGNGYISAAELRHVMTNLGEKLTDEEVDEMIREADIDGDGQVNY |
| | EEFVQMSG |
| 223 | TEEQIAEFKEAFSLFDKDGDGTITTKELGTVMRSLGQNPTEAELQDMINEVDADGNGTIDFPEFLTMM |
| | ARKMKDTDSEEEIREAFRVFDKDGNGYISAAELRHVMTNLGEKLTDEEVDEMIREADIDGDGQVNYE |
| | EFVQMSG |
| 224 | EEQIAEFKEAFSLFDKDGDGTITTKELGTVMRSLGQNPTEAELQDMINEVDADGNGTIDFPEFLTMM |
| | ARKMKDTDSEEEIREAFRVFDKDGNGYISAAELRHVMTNLGEKLTDEEVDEMIREADIDGDGQVNYE |
| | EFVQMSG |
| 225 | EQIAEFKEAFSLFDKDGDGTITTKELGTVMRSLGQNPTEAELQDMINEVDADGNGTIDFPEFLTMMA |
| | RKMKDTDSEEEIREAFRVFDKDGNGYISAAELRHVMTNLGEKLTDEEVDEMIREADIDGDGQVNYEEF |
| | VQMSG |
| 226 | QIAEFKEAFSLFDKDGDGTITTKELGTVMRSLGQNPTEAELQDMINEVDADGNGTIDFPEFLTMMAR |
| | KMKDTDSEEEIREAFRVFDKDGNGYISAAELRHVMTNLGEKLTDEEVDEMIREADIDGDGQVNYEEFV |
| | QMSG |
| 227 | AEFKEAFSLFDKDGDGTITTKELGTVMRSLGQNPTEAELQDMINEVDADGNGTIDFPEFLTMMARK |
| | MKDTDSEEEIREAFRVFDKDGNGYISAAELRHVMTNLGEKLTDEEVDEMIREADIDGDGQVNYEEFV |
| | QMSG |
| 228 | SEEEIREAFRVFDKDGNGYISAAELRHVMTNLGEKLTDEEVDEMIREADIDGDGQVNYEEFVQMMTA |
| | KGGSGADQLTEEQIAEFKEAFSLFDKDGDGTITTKELGTVMRSLGQNPTEAELQDMINEVDADGNGTI |
| | DFPEFLTMMARKMKDTDSG |
| 229 | EEEIREAFRVFDKDGNGYISAAELRHVMTNLGEKLTDEEVDEMIREADIDGDGQVNYEEFVQMMTAK |
| | GGSGADQLTEEQIAEFKEAFSLFDKDGDGTITTKELGTVMRSLGQNPTEAELQDMINEVDADGNGTI |
| | DFPEFLTMMARKMKDTDSG |
| 230 | EEIREAFRVFDKDGNGYISAAELRHVMTNLGEKLTDEEVDEMIREADIDGDGQVNYEEFVQMMTAK |
| | GGSGADQLTEEQIAEFKEAFSLFDKDGDGTITTKELGTVMRSLGQNPTEAELQDMINEVDADGNGTI |
| | DFPEFLTMMARKMKDTDSG |
| 231 | EIREAFRVFDKDGNGYISAAELRHVMTNLGEKLTDEEVDEMIREADIDGDGQVNYEEFVQMMTAKG |
| | GSGADQLTEEQIAEFKEAFSLFDKDGDGTITTKELGTVMRSLGQNPTEAELQDMINEVDADGNGTIDF |
| | PEFLTMMARKMKDTDSG |
| 232 | IREAFRVFDKDGNGYISAAELRHVMTNLGEKLTDEEVDEMIREADIDGDGQVNYEEFVQMMTAKGG |
| | SGADQLTEEQIAEFKEAFSLFDKDGDGTITTKELGTVMRSLGQNPTEAELQDMINEVDADGNGTIDFP |
| | EFLTMMARKMKDTDSG |
| 233 | REAFRVFDKDGNGYISAAELRHVMTNLGEKLTDEEVDEMIREADIDGDGQVNYEEFVQMMTAKGGS |
| | GADQLTEEQIAEFKEAFSLFDKDGDGTITTKELGTVMRSLGQNPTEAELQDMINEVDADGNGTIDFPE |
| | FLTMMARKMKDTDSG |
| 234 | EAFRVFDKDGNGYISAAELRHVMTNLGEKLTDEEVDEMIREADIDGDGQVNYEEFVQMMTAKGGSG |
| | ADQLTEEQIAEFKEAFSLFDKDGDGTITTKELGTVMRSLGQNPTEAELQDMINEVDADGNGTIDFPEF |
| | LTMMARKMKDTDSG |
| 235 | AFRVFDKDGNGYISAAELRHVMTNLGEKLTDEEVDEMIREADIDGDGQVNYEEFVQMMTAKGGSG |
| | ADQLTEEQIAEFKEAFSLFDKDGDGTITTKELGTVMRSLGQNPTEAELQDMINEVDADGNGTIDFPEF |
| | LTMMARKMKDTDSG |
| 236 | VFDKDGNGYISAAELRHVMTNLGEKLTDEEVDEMIREADIDGDGQVNYEEFVQMMTAKGGSGADQ |
| | LTEEQIAEFKEAFSLFDKDGDGTITTKELGTVMRSLGQNPTEAELQDMINEVDADGNGTIDFPEFLTM |
| | MARKMKDTDSG |
| 237 | GNGYISAAELRHVMTNLGEKLTDEEVDEMIREADIDGDGQVNYEEFVQMMTAKGGSGADQLTEEQI |
| | AEFKEAFSLFDKDGDGTITTKELGTVMRSLGQNPTEAELQDMINEVDADGNGTIDFPEFLTMMARK |
| | MKDTDSG |
| 238 | SEEEIREAFRVFDKDGNGYISAAELRHVMTNLGEKLTDEEVDEMIREADIDGDGQVNYEEFVQMMTA |
| | KGGSGADQLTEEQIAEFKEAFSLFDKDGDGTITTKELGTVMRSLGQNPTEAELQDMINEVDADGNGTI |
| | DFPEFLTMMARKMKDTSG |
| 239 | EEEIREAFRVFDKDGNGYISAAELRHVMTNLGEKLTDEEVDEMIREADIDGDGQVNYEEFVQMMTAK |
| | GGSGADQLTEEQIAEFKEAFSLFDKDGDGTITTKELGTVMRSLGQNPTEAELQDMINEVDADGNGTI |
| | DFPEFLTMMARKMKDTSG |
| 240 | EEIREAFRVFDKDGNGYISAAELRHVMTNLGEKLTDEEVDEMIREADIDGDGQVNYEEFVQMMTAK |
| | GGSGADQLTEEQIAEFKEAFSLFDKDGDGTITTKELGTVMRSLGQNPTEAELQDMINEVDADGNGTI |
| | DFPEFLTMMARKMKDTSG |
| 241 | EIREAFRVFDKDGNGYISAAELRHVMTNLGEKLTDEEVDEMIREADIDGDGQVNYEEFVQMMTAKG |
| | GSGADQLTEEQIAEFKEAFSLFDKDGDGTITTKELGTVMRSLGQNPTEAELQDMINEVDADGNGTIDF |
| | PEFLTMMARKMKDTSG |
| 242 | IREAFRVFDKDGNGYISAAELRHVMTNLGEKLTDEEVDEMIREADIDGDGQVNYEEFVQMMTAKGG |
| | SGADQLTEEQIAEFKEAFSLFDKDGDGTITTKELGTVMRSLGQNPTEAELQDMINEVDADGNGTIDFP |
| | EFLTMMARKMKDTSG |
| 243 | REAFRVFDKDGNGYISAAELRHVMTNLGEKLTDEEVDEMIREADIDGDGQVNYEEFVQMMTAKGGS |
| | GADQLTEEQIAEFKEAFSLFDKDGDGTITTKELGTVMRSLGQNPTEAELQDMINEVDADGNGTIDFPE |
| | FLTMMARKMKDTSG |
| 244 | EAFRVFDKDGNGYISAAELRHVMTNLGEKLTDEEVDEMIREADIDGDGQVNYEEFVQMMTAKGGSG |
| | ADQLTEEQIAEFKEAFSLFDKDGDGTITTKELGTVMRSLGQNPTEAELQDMINEVDADGNGTIDFPEF |
| | LTMMARKMKDTSG |
| 245 | AFRVFDKDGNGYISAAELRHVMTNLGEKLTDEEVDEMIREADIDGDGQVNYEEFVQMMTAKGGSG |
| | ADQLTEEQIAEFKEAFSLFDKDGDGTITTKELGTVMRSLGQNPTEAELQDMINEVDADGNGTIDFPEF |
| | LTMMARKMKDTSG |
| 246 | VFDKDGNGYISAAELRHVMTNLGEKLTDEEVDEMIREADIDGDGQVNYEEFVQMMTAKGGSGADQ |
| | LTEEQIAEFKEAFSLFDKDGDGTITTKELGTVMRSLGQNPTEAELQDMINEVDADGNGTIDFPEFLTM |
| | MARKMKDTSG |
| 247 | GNGYISAAELRHVMTNLGEKLTDEEVDEMIREADIDGDGQVNYEEFVQMMTAKGGSGADQLTEEQI |
| | AEFKEAFSLFDKDGDGTITTKELGTVMRSLGQNPTEAELQDMINEVDADGNGTIDFPEFLTMMARK |
| | MKDTSG |
| 248 | SEEEIREAFRVFDKDGNGYISAAELRHVMTNLGEKLTDEEVDEMIREADIDGDGQVNYEEFVQMMTA |
| | KGGSGADQLTEEQIAEFKEAFSLFDKDGDGTITTKELGTVMRSLGQNPTEAELQDMINEVDADGNGTI |
| | DFPEFLTMMARKMKDSG |
| 249 | EEEIREAFRVFDKDGNGYISAAELRHVMTNLGEKLTDEEVDEMIREADIDGDGQVNYEEFVQMMTAK |
| | GGSGADQLTEEQIAEFKEAFSLFDKDGDGTITTKELGTVMRSLGQNPTEAELQDMINEVDADGNGTI |
| | DFPEFLTMMARKMKDSG |
| 250 | EEIREAFRVFDKDGNGYISAAELRHVMTNLGEKLTDEEVDEMIREADIDGDGQVNYEEFVQMMTAK |
| | GGSGADQLTEEQIAEFKEAFSLFDKDGDGTITTKELGTVMRSLGQNPTEAELQDMINEVDADGNGTI |
| | DFPEFLTMMARKMKDSG |
| 251 | EIREAFRVFDKDGNGYISAAELRHVMTNLGEKLTDEEVDEMIREADIDGDGQVNYEEFVQMMTAKG |
| | GSGADQLTEEQIAEFKEAFSLFDKDGDGTITTKELGTVMRSLGQNPTEAELQDMINEVDADGNGTIDF |
| | PEFLTMMARKMKDSG |
| 252 | IREAFRVFDKDGNGYISAAELRHVMTNLGEKLTDEEVDEMIREADIDGDGQVNYEEFVQMMTAKGG |
| | SGADQLTEEQIAEFKEAFSLFDKDGDGTITTKELGTVMRSLGQNPTEAELQDMINEVDADGNGTIDFP |
| | EFLTMMARKMKDSG |
| 253 | REAFRVFDKDGNGYISAAELRHVMTNLGEKLTDEEVDEMIREADIDGDGQVNYEEFVQMMTAKGGS |
| | GADQLTEEQIAEFKEAFSLFDKDGDGTITTKELGTVMRSLGQNPTEAELQDMINEVDADGNGTIDFPE |
| | FLTMMARKMKDSG |
| 254 | EAFRVFDKDGNGYISAAELRHVMTNLGEKLTDEEVDEMIREADIDGDGQVNYEEFVQMMTAKGGSG |
| | ADQLTEEQIAEFKEAFSLFDKDGDGTITTKELGTVMRSLGQNPTEAELQDMINEVDADGNGTIDFPEF |
| | LTMMARKMKDSG |
| 255 | AFRVFDKDGNGYISAAELRHVMTNLGEKLTDEEVDEMIREADIDGDGQVNYEEFVQMMTAKGGSG |
| | ADQLTEEQIAEFKEAFSLFDKDGDGTITTKELGTVMRSLGQNPTEAELQDMINEVDADGNGTIDFPEF |
| | LTMMARKMKDSG |
| 256 | VFDKDGNGYISAAELRHVMTNLGEKLTDEEVDEMIREADIDGDGQVNYEEFVQMMTAKGGSGADQ |
| | LTEEQIAEFKEAFSLFDKDGDGTITTKELGTVMRSLGQNPTEAELQDMINEVDADGNGTIDFPEFLTM |
| | MARKMKDTG |
| 257 | GNGYISAAELRHVMTNLGEKLTDEEVDEMIREADIDGDGQVNYEEFVQMMTAKGGSGADQLTEEQI |
| | AEFKEAFSLFDKDGDGTITTKELGTVMRSLGQNPTEAELQDMINEVDADGNGTIDFPEFLTMMARK |
| | MKDSG |
| 258 | SEEEIREAFRVFDKDGNGYISAAELRHVMTNLGEKLTDEEVDEMIREADIDGDGQVNYEEFVQMMTA |
| | KGGSGADQLTEEQIAEFKEAFSLFDKDGDGTITTKELGTVMRSLGQNPTEAELQDMINEVDADGNGTI |
| | DFPEFLTMMARKMKSG |
| 259 | EEEIREAFRVFDKDGNGYISAAELRHVMTNLGEKLTDEEVDEMIREADIDGDGQVNYEEFVQMMTAK |
| | GGSGADQLTEEQIAEFKEAFSLFDKDGDGTITTKELGTVMRSLGQNPTEAELQDMINEVDADGNGTI |
| | DFPEFLTMMARKMKSG |
| 260 | EEIREAFRVFDKDGNGYISAAELRHVMTNLGEKLTDEEVDEMIREADIDGDGQVNYEEFVQMMTAK |
| | GGSGADQLTEEQIAEFKEAFSLFDKDGDGTITTKELGTVMRSLGQNPTEAELQDMINEVDADGNGTI |
| | DFPEFLTMMARKMKSG |
| 261 | EIREAFRVFDKDGNGYISAAELRHVMTNLGEKLTDEEVDEMIREADIDGDGQVNYEEFVQMMTAKG |
| | GSGADQLTEEQIAEFKEAFSLFDKDGDGTITTKELGTVMRSLGQNPTEAELQDMINEVDADGNGTIDF |
| | PEFLTMMARKMKSG |
| 262 | IREAFRVFDKDGNGYISAAELRHVMTNLGEKLTDEEVDEMIREADIDGDGQVNYEEFVQMMTAKGG |
| | SGADQLTEEQIAEFKEAFSLFDKDGDGTITTKELGTVMRSLGQNPTEAELQDMINEVDADGNGTIDFP |
| | EFLTMMARKMKSG |
| 263 | REAFRVFDKDGNGYISAAELRHVMTNLGEKLTDEEVDEMIREADIDGDGQVNYEEFVQMMTAKGGS |
| | GADQLTEEQIAEFKEAFSLFDKDGDGTITTKELGTVMRSLGQNPTEAELQDMINEVDADGNGTIDFPE |
| | FLTMMARKMKSG |
| 264 | EAFRVFDKDGNGYISAAELRHVMTNLGEKLTDEEVDEMIREADIDGDGQVNYEEFVQMMTAKGGSG |
| | ADQLTEEQIAEFKEAFSLFDKDGDGTITTKELGTVMRSLGQNPTEAELQDMINEVDADGNGTIDFPEF |
| | LTMMARKMKSG |
| 265 | AFRVFDKDGNGYISAAELRHVMTNLGEKLTDEEVDEMIREADIDGDGQVNYEEFVQMMTAKGGSG |
| | ADQLTEEQIAEFKEAFSLFDKDGDGTITTKELGTVMRSLGQNPTEAELQDMINEVDADGNGTIDFPEF |
| | LTMMARKMKSG |
| 266 | VFDKDGNGYISAAELRHVMTNLGEKLTDEEVDEMIREADIDGDGQVNYEEFVQMMTAKGGSGADQ |
| | LTEEQIAEFKEAFSLFDKDGDGTITTKELGTVMRSLGQNPTEAELQDMINEVDADGNGTIDFPEFLTM |
| | MARKMKTG |
| 267 | GNGYISAAELRHVMTNLGEKLTDEEVDEMIREADIDGDGQVNYEEFVQMMTAKGGSGADQLTEEQI |
| | AEFKEAFSLFDKDGDGTITTKELGTVMRSLGQNPTEAELQDMINEVDADGNGTIDFPEFLTMMARK |
| | MKSG |
| 268 | SEEEIREAFRVFDKDGNGYISAAELRHVMTNLGEKLTDEEVDEMIREADIDGDGQVNYEEFVQMMTA |
| | KGGSGADQLTEEQIAEFKEAFSLFDKDGDGTITTKELGTVMRSLGQNPTEAELQDMINEVDADGNGTI |
| | DFPEFLTMMARKMSG |
| 269 | EEEIREAFRVFDKDGNGYISAAELRHVMTNLGEKLTDEEVDEMIREADIDGDGQVNYEEFVQMMTAK |
| | GGSGADQLTEEQIAEFKEAFSLFDKDGDGTITTKELGTVMRSLGQNPTEAELQDMINEVDADGNGTI |
| | DFPEFLTMMARKMSG |
| 270 | EEIREAFRVFDKDGNGYISAAELRHVMTNLGEKLTDEEVDEMIREADIDGDGQVNYEEFVQMMTAK |
| | GGSGADQLTEEQIAEFKEAFSLFDKDGDGTITTKELGTVMRSLGQNPTEAELQDMINEVDADGNGTI |
| | DFPEFLTMMARKMSG |
| 271 | EIREAFRVFDKDGNGYISAAELRHVMTNLGEKLTDEEVDEMIREADIDGDGQVNYEEFVQMMTAKG |
| | GSGADQLTEEQIAEFKEAFSLFDKDGDGTITTKELGTVMRSLGQNPTEAELQDMINEVDADGNGTIDF |
| | PEFLTMMARKMSG |
| 272 | IREAFRVFDKDGNGYISAAELRHVMTNLGEKLTDEEVDEMIREADIDGDGQVNYEEFVQMMTAKGG |
| | SGADQLTEEQIAEFKEAFSLFDKDGDGTITTKELGTVMRSLGQNPTEAELQDMINEVDADGNGTIDFP |
| | EFLTMMARKMSG |
| 273 | REAFRVFDKDGNGYISAAELRHVMTNLGEKLTDEEVDEMIREADIDGDGQVNYEEFVQMMTAKGGS |
| | GADQLTEEQIAEFKEAFSLFDKDGDGTITTKELGTVMRSLGQNPTEAELQDMINEVDADGNGTIDFPE |
| | FLTMMARKMSG |
| 274 | EAFRVFDKDGNGYISAAELRHVMTNLGEKLTDEEVDEMIREADIDGDGQVNYEEFVQMMTAKGGSG |
| | ADQLTEEQIAEFKEAFSLFDKDGDGTITTKELGTVMRSLGQNPTEAELQDMINEVDADGNGTIDFPEF |
| | LTMMARKMSG |
| 275 | AFRVFDKDGNGYISAAELRHVMTNLGEKLTDEEVDEMIREADIDGDGQVNYEEFVQMMTAKGGSG |
| | ADQLTEEQIAEFKEAFSLFDKDGDGTITTKELGTVMRSLGQNPTEAELQDMINEVDADGNGTIDFPEF |
| | LTMMARKMSG |
| 276 | VFDKDGNGYISAAELRHVMTNLGEKLTDEEVDEMIREADIDGDGQVNYEEFVQMMTAKGGSGADQ |
| | LTEEQIAEFKEAFSLFDKDGDGTITTKELGTVMRSLGQNPTEAELQDMINEVDADGNGTIDFPEFLTM |
| | MARKMTG |
| 277 | GNGYISAAELRHVMTNLGEKLTDEEVDEMIREADIDGDGQVNYEEFVQMMTAKGGSGADQLTEEQI |
| | AEFKEAFSLFDKDGDGTITTKELGTVMRSLGQNPTEAELQDMINEVDADGNGTIDFPEFLTMMARK |
| | MSG |
| 278 | SDQLTEEQIAEFKEAFSLFDKDGDGTITTKELGTVMRSLGQNPTEAELQDMINEVDADGNDTIDFPEFL |
| | TMMARKMKDTDSEEEIREAFRVFDKDGNGYISAAELRHVMTNLGEKLTDEEVDEMIREADNDGDG |
| | QVNYEEFVQMMTAKGGSG |
| 279 | GTITTKELGTVMRSLGQNPTEAELQDMINEVDSDGNGTIDFPEFLTMMARKMKDTDSEEEIREAFRV |
| | FDKDGNGYISAAELRHVMTNLGEKLTDEEVDEMIREADIDGDGQVNYEEFVK |
| 280 | ADQLTEEQVAEFKEAFSLFDKDGDGTITTKELGTVMRSLGQNPTEAELQDMINEVDADGNGTIDFPEF |
| | LTMMARKMMDTDSEEEIREAFRVFDKDGNGYISAAELRHVMTNLGEKLTDEEVDEMIREADIDGDG |
| | QVNYEEFVQMMTAKGGSG |
| 281 | GTITTKELGTVMRSLGQNPTEAELQDMINEVDADGNGTIDFPEFLTMMARKMKDTDSGEEIREAFRV |
| | FDKDGNGYISAAELRHVMTNLGEKLTDEEVDEMIREADIDGDGQVNYEEFVK |
| 282 | GTITTKELGTVMRSLGQNPTEAELQDMINEVDADGNGTIDFPEFLTMMARKMKDTDSEEEIREAFRV |
| | FDKDGNGYISAAELRHVMTNLGEKLTDEEVDEMIREADIDDDGQVNYEEFVK |
| 283 | GTITTKELGTVMRSLGQNPTEAELQDMINEVDADGNGTIDFPEFLTMMARKMKDTDSEEEIREAFRV |
| | FDKDGNGYISAAELRHVMTNLSEKLTDEEVDEMIREADIDGDGQVNYEEFVK |
| 284 | GTITTKELGTVMRSLGQNPTEAELQDMIIEVDADGNGTIDFPEFLTMMARKMKDTDSEEEIREAFRVF |
| | DKDGNGYISAAELRHVMTNLGEKLTDEEVDEMIREADIDGDGQVNYEEFVK |
| 285 | GMITTKELGTVMRSLGQNPTEAELQDMINEVDADGNGTIDFPEFLTMMTRKMKDTDSEEEIREVFRV |
| | FDKDGNGYISAAELRHVMTNLGEKLTDEEVDEMIREADIGGDGQVNYEEFVK |
| 286 | GTITTKELGTVMRSLGQNPTEVELQDMINEVDADGNGTIDFPEFLTMMARKMKDTDSEEEIREAFRV |
| | FDKDGNGYISAAELRHVMTNLGEKLTDEEVDEMIREADIDGEGQVNYEEFVK |
| 287 | GTITTKELGTVMRSLGQNPTEAELQDMINEVDADGNDTIDFPEFLTMMARKMKDTDSEEEIREAFRV |
| | FDKDGNGYISAAELRHVMTNLGEKLTDEEVDEMIREADIDGDGQVNYEEFVK |

In some embodiments, the first binding domain of the provided bispecific molecule comprises a linker containing a calmodulin partial sequence that is homologous to a polypeptide sequence disclosed in any one of SEQ ID NOS:99-287. In some embodiments, the first binding domain comprises a linker containing a sequence that has at least 65%, 70%, 75%, 80%, 85%, 90%, 95%, 96%, 97%, 98% or 99% identity to the linker sequence disclosed in any one of SEQ ID NOS:99-287 the first binding domain comprises a linker containing a sequence that contains 1-25, 1-20, 1-15, 1-10, 1-5 or 5-20 amino acid additions, substitutions, and/or deletions compared to disclosed in any one of SEQ ID NOS:99-287.

In some embodiments, the provided bispecific molecule contains a switch linker sequence listed in U.S. Pat. No. 10,730,922, or a sequence derived from such linker that contains one, two, three, four or more amino acid additions, deletions, or substitutions that collectively result in an increased allosteric switch effect compared to such parental switch linker.

In some embodiments, bispecific constructs contain a switch linker containing a sequence that has at least to 65%, 70%, 75%, 80%, 85%, 90%, 95%, or more than 95% identity to a calmodulin linker sequence disclosed U.S. Pat. No. 10,730,922, the contents of which are herein incorporated by reference in its entirety.

### Polynucleotides, Host Cells and Methods of Production

The disclosure also provides polynucleotides encoding the bispecific molecules provided herein. In some embodiments, the polynucleotides are provided as DNA constructs. In other embodiments, the polynucleotides are provided as messenger RNA transcripts.

The polynucleotides can be constructed by known methods such as by combining the genes encoding the three binding domains either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. In the embodiments where the target antigen binding domain is a small molecule, the polynucleotides contain genes encoding the CD3 binding domain and the half-life extension domain. In the embodiments where the half-life extension domain is a small molecule, the polynucleotides contain genes encoding the domains that bind to CD3 and the target antigen. Depending on the vector system and host utilized, any number of suitable transcription and translation elements, including constitutive and inducible promoters, may be used. The promoter is selected such that it drives the expression of the polynucleotide in the respective host cell.

In some embodiments, the polynucleotide(s) is inserted into one or more vectors. In particular embodiments, the polynucleotide(s) is inserted into one or more expression vectors. The recombinant vector can be constructed according to known methods using known vectors. Vectors of particular interest include plasmids, phagemids, phage derivatives, virii (e.g., retroviruses, adenoviruses, adeno-associated viruses, herpes viruses, lentiviruses, and the like), and cosmids.

In some embodiments, the provided polynucleotides and/or vectors are contained in a host cell, optionally a host cell chosen for expression. The host cell may be of prokaryotic or eukaryotic origin. In some embodiments, the host cell is selected from COS-1, COS-7, HEK293, BHK2, CHO, ExpiCHO and CHO-K1, CHO-S, CHO-DG44, BSC-1, He G2, S2/0, HeLa, myeloma, lymphoma, yeast, insect or plant cells, or any derivative, immortalized or transformed cell thereof. In some embodiments, the host cell is selected from a species or organism incapable of glycosylating polypeptides, e.g., a prokaryotic cll′ or organism, such as BL21, BL21(DE3), BL21-GOLD(DE3), XL-Blue, JM109.HMS174, HMS114(DE3), and any of the natural or engineered E coli or Pseudomonas strains.

A variety of expression vector/host systems may be utilized to contain and express the polynucleotide(s) encoding the polypeptide(s) of the provided bispecific molecules. Examples of expression vectors for expression in mammalian cells, PICHIAPINK™ Yeast Expression Systems (Invitrogen), BACUVANCE™ Baculovirus Expression System (GenScript), GS expression vectors (Lonza), and the vectors used in the selection system of the SUREtechnology Platform™ (Selexis), Leap in transposase (ATUM), DirectedLuck transposase (Probiogen), and GPEx Lightning (Catalent) to name a few. Other host expression systems are known in the art and can routinely be adapted and used by one skilled in the art to express the provided multispecific molecules.

In some embodiments, a provided bispecific molecule is produced by introducing a vector or vectors encoding the bispecific molecule as described above into a host cell (e.g., by transformation or transfection) and culturing the host cell under conditions suitable for expressing the encoded protein. The expressed protein can then be isolated using techniques know in the art, and optionally, further purified using known techniques.

In some embodiments, the disclosure provides a host cell comprising a polynucleotide or polynucleotides encoding a provided bispecific molecule.

In some embodiments, a provided bispecific molecule is produced by culturing a host cell comprising polynucleotide(s) encoding the bispecific molecule under conditions suitable for expressing the encoded protein. In further embodiments, the expressed protein is isolated. In additional embodiments the expressed protein is further purified.

In some embodiments, the process comprises culturing a host transformed or transfected with a vector or vectors comprising a polynucleotide or polynucleotides encoding a provided bispecific molecule under conditions suitable for expressing the bispecific molecule. In further embodiments, the expressed protein is isolated. In additional embodiments the expressed protein is further purified.

### Switch Ligands

In one embodiment, the switch ligand used to regulate the affinity of the scFv carrying the switch linker are selected from the peptides listed in Table 3 (SEQ ID NO:1-SEQ ID NO:85). Other peptides which bind to calmodulin can be used as well.

TABLE 3
Switch ligands (peptides) that bind to the switch linker.

| | | |
|---|---|---|
| SEQ ID NO: 1 | skMLCK (M13 wt) | KRRWKKNFIAVSAANRFKKISSSGAL |
| SEQ ID NO: 2 | M13-Peptid S | KRRWKKNFIAVSAANRFKKISSSGA |
| SEQ ID NO: 3 | M13-Peptid XS | RWKKNFIAVSAANRFKKIS |
| SEQ ID NO: 4 | M13-Var2 | WKKNFIAVSAANRFKKIS |
| SEQ ID NO: 5 | M13-Var3 | RWKKNFIAVSAANRFKKI |
| SEQ ID NO: 6 | M13-Var4 | RWKKNFIAVSAANRFKK |
| SEQ ID NO: 7 | M13-Var5 | RWKKAFIAVSAANRFKKIS |
| SEQ ID NO: 8 | M13-Var6 | WKKAFIAVSAANRFKKIS |
| SEQ-ID NO: 9 | M13-Var7 | RWKKAFIAVSAANRFKKI |
| SEQ ID NO: 10 | M13-Var8 | RWKKAFIAVSAANRFKK |
| SEQ ID NO: 11 | M13-Var9 | RWKKNIIAVSAANRFKKIS |
| SEQ ID NO: 12 | M13-Var10 | RWKKNFIKVSAANRFKKIS |
| SEQ ID NO: 13 | M13-Var11 | RWKKNFIAVLAANRFKKIS |
| SEQ ID NO: 14 | M13-Var12 | RWKKNFIAVSAINRFKKIS |
| SEQ ID NO: 15 | M13-Var13 | RWKKAIIKVLAINRFKKIS |
| SEQ ID NO: 16 | M13-Var14 | RWKKAIIKVLAANRFKKIS |
| SEQ ID NO: 17 | M13-Var15 | RWKKAIIKVSAANRFKKIS |
| SEQ ID NO: 18 | M13-Var16 | RWKKAIIAVSAANRFKKIS |
| SEQ ID NO: 19 | M13-Var17 | RWKKAFIKVLAINRFKKIS |
| SEQ ID NO: 20 | M13-Var18 | RWKKAFIAVLAINRFKKIS |
| SEQ ID NO: 21 | M13-Var19 | RWKKAFIAVSAINRFKKIS |
| SEQ ID NO: 22 | M13-Var20 | RWKKNIIKVLAINRFKKIS |
| SEQ ID NO: 23 | M13-Var21 | RWKKNFIKVLAINRFKKIS |
| SEQ ID NO: 24 | M13-Var22 | RWKKNFIAVLAINRFKKIS |
| SEQ ID NO: 25 | M13-Var23 | RWKKAIIKVSAINRFKKIS |
| SEQ ID NO: 26 | M13-Var24 | RWKKAIIAVSAINRFKKIS |
| SEQ ID NO: 27 | M13-Var25 | RWKKAIIAVLAINRFKKIS |
| SEQ ID NO: 28 | M13-Var26 | RWKKAFIKVSAANRFKKIS |
| SEQ ID NO: 29 | M13-Var27 | RWKKAFIAVLAANRFKKIS |
| SEQ ID NO: 30 | M13-Var28 | RWKKAIIAVLAANRFKKIS |
| SEQ ID NO: 31 | M13-Var29 | RWKKAFIKVLAANRFKKIS |
| SEQ ID NO: 32 | M13-Var30 | RWKKAFIKVSAINRFKKIS |
| SEQ ID NO: 33 | M13-Var31 | RWKKNIIKVLAANRFKKIS |
| SEQ ID NO: 34 | M13-Var32 | RWKKNIIKVSAANRFKKIS |
| SEQ ID NO: 35 | M13-Var33 | RWKKNIIAVLAINRFKKIS |
| SEQ ID NO: 36 | M13-Var34 | RWKKNIIAVLAANRFKKIS |
| SEQ ID NO: 37 | M13-Var35 | RWKKNIIAVSAINRFKKIS |
| SEQ ID NO: 38 | M13-Var36 | RWKKNDIAVSAANRFKKIS |
| SEQ ID NO: 39 | M13-Var37 | RWKKNFIDVSAANRFKKIS |
| SEQ ID NO: 40 | M13-Var38 | RWKKNFIAVHAANRFKKIS |
| SEQ ID NO: 41 | M13-Var39 | RWKKNFIAVSARNRFKKIS |
| SEQ ID NO: 42 | CBP-Var2 | GVRNIKSMWEKGNVFSS |
| SEQ ID NO: 43 | CBP-Var3 | ARRKLKAAVKAVVASSRLGS |
| SEQ ID NO: 44 | CBP-Var4 | FMNNWEVYKLLAHIRPPAPKSGSYT |
| SEQ ID NO: 45 | CBP-Var5 | ARKEVIRNKIRAIGKMARVFSVLR |
| SEQ ID NO: 46 | CBP-Var6 | RGQILWFRGLNRIQTQIKVVNAFS |
| SEQ ID NO: 47 | CBP-Var7 | RRKHLQRPIFRLRCLVKOLEK PDE |
| SEQ ID NO: 48 | CBP-Var8 | KTASPWKSARLMVHTVATFNSIKE |
| SEQ ID NO: 49 | CBP-Var9 | KKKKKRFSFKKSFKLSGFSFKKSKK |
| SEQ ID NO: 50 | CBP-Var10 | KKKATFRAITSTLASSFKRRRSSK |
| SEQ ID NO: 51 | CBP-Var11 | KRRAIGFKKLAEAVKFSAKLMGQ |
| SEQ ID NO: 52 | CBP-Var12 | RKKTFKEVANAVKISASLMG |
| SEQ ID NO: 53 | CBP-Var13 | AAARKEVIRNKIRAIGKMARVFSVL |
| SEQ ID NO: 54 | CBP-Var14 | KIQASFRGHMARKKIKSGE |
| SEQ ID NO: 55 | CBP-Var15 | KAATKIQASFRGHITRKKLKGEKKG |
| SEQ ID NO: 56 | CBP-Var16 | LRRGQILWFRGLNRIQTQIK |
| SEQ ID NO: 57 | CBP-Var17 | SKYITTIAGVMTLSQVKGFVRKNGV |
| SEQ ID NO: 58 | CBP-Var18 | ATLDALLAALRRIQRAD |
| SEQ ID NO: 59 | CBP-Var19 | RAANLWPSPLMIKRSKKNS |
| SEQ ID NO: 60 | CBP-Var20 | KIYAAMMIMEYYRQSKAKKLQ |
| SEQ ID NO: 61 | CBP-Var21 | KIYAAMMIMDYYKQSKVKKQR |
| SEQ ID NO: 62 | CBP-Var22 | KFYATFLIQEHFRKFMKRQEE |
| SEQ ID NO: 63 | CBP-Var23 | GTGAALSWQAAIDAARQAKLMGSA |
| SEQ ID NO: 64 | CBP-Var24 | HMGKVYAALMIFDFYKQNKTSRD |
| SEQ ID NO: 65 | CBP-Var25 | GHMGKIYAAMMIMDYYKQSKVKK |
| SEQ ID NO: 66 | CBP-Var26 | HMGKIYAAMMIMEYYRQSKAKK |
| SEQ ID NO: 67 | CBP-Var27 | IKKNFAKSKWKQAFNATAVVRHMRK |
| SEQ ID NO: 68 | CBP-Var28 | LKKFNARRKLKGAILTTMLATRNFS |
| SEQ ID NO: 69 | CBP-Var29 | HSMQALSWRKLYLSRAKLKA |
| SEQ ID NO: 70 | CBP-Var30 | AKSKWKQAFNATAVVRHMRKLQ |
| SEQ ID NO: 71 | smMLCK | ARRKWQKTGHAVRAIGRLSS |
| SEQ ID NO: 72 | CaMKII | ARRKLKGAILTTMLATRNFS |
| SEQ ID NO: 73 | Caldesmon | GVRNIKSMWEKGNVFSS |
| SEQ ID NO: 74 | Calspermin | ARRKLKAAVKAVVASSRLGS |
| SEQ ID NO: 75 | Phosphofructokinase | FMNNWEVYKLLAHIRPPAPKSGSYTV |
| SEQ ID NO: 76 | Calcineurin | ARKEVIRNKIRAIGKMARVFSVLR |
| SEQ ID NO: 77 | Ca2+-ATPase | RGQILWFRGLNRIQTQIKVVNAFS |
| SEQ ID NO: 78 | PDE | RRKHLQRPIFRLRCLVKOLEK |
| SEQ ID NO: 78 | cNOS | KRRAIGFKKLAEAVKFSAKLMGQ |
| SEQ ID NO: 80 | Neuromodulin | KAHKAATKIQASFRGHITRKKLKGEKK |
| SEQ ID NO: 81 | a-Spektrin | KTASPWKSARLMVHTVATFNSIKE |
| SEQ ID NO: 82 | MARCKS | KKKKKRFSFKKSFKLSGFSFKKSKK |
| SEQ ID NO: 83 | Mastoparan | INLKALAALAKKIL |
| SEQ ID NO: 84 | Mellitin | GIGGAVLKVLTTGLPALISWIKRKR |
| SEQ ID NO: 85 | PM | VDWKKIGQHILSVL |

In some embodiments, peptides which can act as switch ligands can be obtained by screening of large repertoires of peptides using in vitro selection methods like phage display, yeast display, ribosomal display, cell display or other selection systems to identify binders from combinatorial libraries. In one embodiment, homologous peptides (e.g., polypeptides having at least 75%, 80%, 85%, 90% 95%, 96%, 97%, 98%, or 99% sequence identity with a reference polypeptide sequence) of one of the Sequences with SEQ ID NO:1-85 are generated by mutagenesis or combinatorial DNA synthesis of the DNA sequence encoding such peptides. The mutagenized or synthesized DNA is then inserted into a phage display vector and the resulting phage are used in an affinity panning on a calmodulin variant or a protein containing a switch linker using the method described in Chakravarty et al., FEBS Letters 476:296-300 (2000). After washing of unbound phage, the bound phage are eluted, allowing the encoded peptides to be selected for higher affinity to calmodulin or a protein containing a switch linker. Phage display panning methods known to those skilled in the art, for example as reviewed in Frenzel et al., (Transfus. Med. Hemother. 44:312-318 (2017); doi: 10.1159/000479633) can be employed, allowing to adapt the biochemical milieu during the panning step for efficient selection. In some embodiments, mutation libraries of peptides which can act as switch ligands can be generated by the peptide SPOT method on membranes, or peptides can be printed by laser printers or other array generation methods known as state of the art to solid supports and screened for better binding by adding enzymatically or fluorescently labelled calmodulin or calmodulin detected by other means for signal generation.

Peptides with an affinity to calmodulin of at least 2 fold, more than 3 fold, more than 5 fold or more than 10 fold better than the original peptide are preferably used as switch ligands.

In some embodiments, one of the peptides shown in Table 3 are administered to patients which have been administered a bispecific antibody containing a scFv carrying a switch linker for allosteric modulation.

In one embodiment, the switch ligand used to regulate the affinity of the scFv carrying the switch linker is selected from the list of substances shown in Table 4.

TABLE 4
Exemplary switch ligands

| | |
|---|---|
| Calmidazolium | (1-[bis(4-chlorophenyl)methyl]-3-[2-(2,4-dichlorophenyl)-2-[(2,4- |
| (CZ) | dichlorophenyl)methoxy]ethyl]imidazol-3-ium) |
| Trifluoperazine | 10-[3-(4-methylpiperazin-1-yl)propyl]-2-(trifluoromethyl) |
| | phenothiazine |
| Tamoxifen (TMX) | (Z)-2-[4-(1,2-Diphenylbut-1-enyl)phenoxy]-N,N-dimethyl-ethanamine |
| Metixene | 1-methyl-3-(9H-thioxanthen-9-ylmethyl)piperidine |
| Carbamazepine | benzo[b][1]benzazepine-11-carboxamide |
| Amitriptyline | N,N-dimethyl-3-(2-tricyclo[9.4.0.03,8]pentadeca-1(15),3,5,7,11,13- |
| | hexaenylidene)propan-1-amine |
| Amoxapine | 8-chloro-6-piperazin-1-ylbenzo[b][1,4]benzoxazepine |
| Clomipramine | 3-(2-chloro-5,6-dihydrobenzo[b][1]benzazepin-11-yl)-N,N- |
| | dimethylpropan-1-amine |
| Desipramine | 3-(5,6-dihydrobenzo[b][1]benzazepin-11-yl)-N-methylpropan-1- |
| | amine |
| Dibenzepin | 5-[2-(dimethylamino)ethyl]-11-methylbenzo[b][1,4] benzodiazepin-6- |
| | one |
| Lofepramine | 1-(4-chlorophenyl)-2-[3-(5,6-dihydrobenzo[b][1]benzazepin-11- |
| | yl)propyl-methylamino]ethanone |
| Maprotiline | N-methyl-3-(1-tetracyclo[6.6.2.02,7.09,14]hexadeca-2,4,6,9,11,13- |
| | hexaenyl)propan-1-amine |
| Mianserin | 5-methyl-2,5-diazatetracyclo[13.4.0.02,7.08,13]nonadeca- |
| | 1(19),8,10,12,15,17-hexaene |
| Nortriptyline | N-methyl-3-(2-tricyclo[9.4.0.03,8]pentadeca-1(15),3,5,7,11,13- |
| | hexaenylidene)propan-1-amine |
| Opipramol | 2-[4-(3-benzo[b][1]benzazepin-11-ylpropyl)piperazin-1-yl]ethanol |
| Paroxetine | (3S,4R)-3-(1,3-benzodioxol-5-yloxymethyl)-4-(4-fluorophenyl) |
| | piperidine |
| Protriptyline | N-methyl-3-(2-tricyclo[9.4.0.03,8]pentadeca-1(15),3,5,7,9,11,13- |
| | heptaenyl)propan-1-amine |
| Trimipramine | 3-(5,6-dihydrobenzo[b][1]benzazepin-11-yl)-N,N,2-trimethylpropan- |
| | 1-amine |
| Chlorprothixene | (3Z)-3-(2-chlorothioxanthen-9-ylidene)-N,N-dimethylpropan-1-amine |
| Chlorpromazine | 3-(2-chlorophenothiazin-10-yl)-N,N-dimethylpropan-1-amine |
| Clozapine | 3-chloro-6-(4-methylpiperazin-1-yl)-11H-benzo[b][1,4] |
| | benzodiazepine |
| Clozapine N-oxide | 3-chloro-6-(4-methyl-4-oxidopiperazin-4-ium-1-yl)-11H- |
| | benzo[b][1,4]benzodiazepine |
| N-Desmethyl- | 3-chloro-6-piperazin-1-yl-11H-benzo[b][1,4]benzodiazepine |
| clozapine | Fluphenazine |
| Haloperidol | 4-[4-(4-chlorophenyl)-4-hydroxypiperidin-1-yl]-1-(4- |
| | fluorophenyl)butan-1-one |
| Levomepromazine | (2R)-3-(2-methoxyphenothiazin-10-yl)-N,N,2-trimethylpropan-1- |
| | amine |
| Loxapine | 8-chloro-6-(4-methylpiperazin-1-yl)benzo[b][1,4] benzoxazepine |
| Mesoridazine | 10-[2-(1-methylpiperidin-2-yl)ethyl]-2-methylsulfinyl-phenothiazine |
| Olanzapine | 2-methyl-4-(4-methylpiperazin-1-yl)-10H-thieno[2,3- |
| | b][1,5]benzodiazepine |
| Penfluridol | 1-[4,4-bis(4-fluorophenyl)butyl]-4-[4-chloro-3-(trifluoro- |
| | methyl)phenyl]piperidin-4-ol |
| Perphenazine | 2-[4-[3-(2-chlorophenothiazin-10-yl)propyl]piperazin-1-yl]ethanol |
| Prochlorperazine | 2-chloro-10-[3-(4-methylpiperazin-1-yl)propyl]phenothiazine |
| Promethazine | N,N-dimethyl-1-phenothiazin-10-ylpropan-2-amine |
| Risperidone | 3-[2-[4-(6-fluoro-1,2-benzoxazol-3-yl)piperidin-1-yl]ethyl]-2-methyl- |
| | 6,7,8,9-tetrahydropyrido[1,2-a]pyrimidin-4-one |
| Sulforidazine | 10-[2-(1-methylpiperidin-2-yl)ethyl]-2-methylsulfonylphenothiazine |
| Thioridazine | 10-[2-(1-methylpiperidin-2-yl)ethyl]-2-methylsulfanyl-phenothiazine |
| Diltiazem | [(25,3S)-5-[2-(dimethylamino)ethyl]-2-(4-methoxyphenyl)-4-oxo-2,3- |
| | dihydro-1,5-benzothiazepin-3-yl] acetate |
| Felodipine | 5-O-ethyl 3-O-methyl 4-(2,3-dichlorophenyl)-2,6-dimethyl-1,4- |
| | dihydropyridine-3,5-dicarboxylate |
| Verapamil | 2-(3,4-dimethoxyphenyl)-5-[2-(3,4-dimethoxyphenyl)ethyl- |
| | methylamino]-2-propan-2-ylpentanenitrile |
| Alexidine | 1-[N′-[6-[[amino-[N′-(2-ethylhexyl)carbamimidoyl]- |
| | amino]methylidene]amino]hexyl]carbamimidoyl]-2-(2- |
| | ethylhexyl)guanidine |
| Benzethonium | benzyl-dimethyl-[2-[2-[4-(2,4,4-trimethylpentan-2- |
| | yl)phenoxy]ethoxy]ethyl]azanium |
| Otilonium | diethyl-methyl-[2-[4-[(2-octoxybenzoyl)amino]- |
| | benzoyl]oxyethyl]azanium |
| Thonzonium | hexadecyl-[2-[(4-methoxyphenyl)methyl-pyrimidin-2-ylamino]ethyl]- |
| | dimethylazanium |
| Cetrimonium | hexadecyl(trimethyl)azanium |
| Domiphen | dodecyl-dimethyl-(2-phenoxyethyl)azanium |
| W7 | N-(6-Aminohexyl)-5-chloro-1-naphthalenesulfonamide hydrochloride |
| W13 | N-(4-Aminobutyl)-5-chloro-2-naphthalenesulfonamide |
| A-7 hydrochloride | N-(10-aminodecyl)-5-chloronaphthalene-1-sulfonamide; |
| | hydrochloride |
| W5 | N-(6-Aminohexyl)-1-naphthalenesulfonamide Hydrochloride |
| CGS 9343B | 1,3-Dihydro-1-[1-[(4-methyl-4H,6H-pyrrolo[1,2-a][4,1]- |
| | benzoxazepin-4-yl)methyl]-4-piperidinyl]-2H-benzimidazol-2-one |
| | maleate |
| CAS 118896-95-0 | N-(5-Aminopentyl)-5-chloro-2-naphthalenesulfonamide |
| Ophiobolin A | (18R)-14,18-Epoxy-3-hydroxy-5-oxoophiobola-7,19-dien-25-al;(18R)- |
| | 3-Hydroxy-5-oxo-14,18-epoxyophiobola-7,19-diene-25-al or |
| | Cochliobolin A |
| Compound 48/80 | (Poly({2-methoxy-5-[2-(methylamino)ethyl]-1,3-phenylene} |
| | methylene)) |
| E6 Berbamine | 6,6′,7-Trimethoxy-2,2′-dimethylberbaman-12-yl acetate |
| Polistes | (3S)-4-[[(2S)-1-[[(2S)-6-amino-1-[[(2S)-6-amino-1-[[(25,3S)-1-[[2- |
| mastoparan | [[(2S)-5-amino-1-[[(2S)-1-[[(25,3S)-1-[[(2S)-1-[[(2S)-1-[[(2S)-1-[[(2S)-1- |
| | amino-4-methyl-1-oxopentan-2-yl]amino]-3-methyl-1-oxobutan-2- |
| | yl]amino]-3-hydroxy-1-oxopropan-2-yl]amino]-4-methyl-1- |
| | oxopentan-2-yl]amino]-3-methyl-1-oxopentan-2-yl]amino]-3-(1H- |
| | imidazol-5-yl)-1-oxopropan-2-yl]amino]-1,5-dioxopentan-2- |
| | yl]amino]-2-oxoethyl]amino]-3-methyl-1-oxopentan-2-yl]amino]-1- |
| | oxohexan-2-yl]amino]-1-oxohexan-2-yl]amino]-3-(1H-indol-3-yl)-1- |
| | oxopropan-2-yl]amino]-3-[[(2S)-2-amino-3-methylbutanoyl]amino]-4- |
| | oxobutanoic acid |

In one embodiment, the switch ligand used to regulate the affinity of the scFv carrying the switch linker is a Calmodulin antagonist, for example a Calmodulin antagonist that is approved for clinical use in other indications.

In some embodiments, one of the substances shown in Table 3 or 4 are administered to patients which have been treated with a bispecific antibody containing a scFv carrying a switch linker for allosteric modulation.

In some embodiments, small molecules which can act as switch ligands can be obtained by screening of compounds that are obtained from natural sources (e.g., animals, plants, and microorganisms), and their physiological processes as well as drug metabolites produced from exogenous drug applications of which prospective lead compounds can be further screened and optimized via observations of their binding affinity to calmodulin and/or their influence on calmodulin induced affinity changes of the antibody scFv for its cognate antigen. Methodologies for screening of lead compounds for desirable characteristics is well known to those skilled in the art examples of which are as reviewed in Ma et al., Am. J. Transl. Res. 13(3): 853-870 (2021).

In some embodiments, the bispecific molecule containing a switchable scFv which has reduced affinity for its target (e.g., CD3) in the absence of switch ligand has been engineered for a long pharmacokinetic half-life (e.g., by fusing to human serum albumin (HSA) or Fc such that when administered to the patient it is not rapidly cleared from the circulation but rather binds and accumulates at the site of the tumor without recruiting T cells until acted upon by the switch ligand that increases its affinity for the target (e.g., CD3) and thus recruits T-cells following administration of the switch ligand to the patient.

In one embodiment, the switch ligand has a short pharmacokinetic half-life such that when administered to the patient it is rapidly cleared from the body thus forming a means of rapid and temporal affinity increases or decreases of the switchable scFv component of the bispecific molecule and thus resulting in the control of the bispecific molecule when both are administered.

In some embodiments, the switch ligand is formulated for long duration controlled release using a biodegradable polymer or nanoparticle such as a hydrogel e.g., PEG-PLA.

In some embodiments, the bispecific molecule containing a switchable scFv which has reduced affinity for its target (e.g., CD3) in the presence of switch ligand has been engineered for a long pharmacokinetic half-life (e.g., by fusing to human serum albumin (HSA) or Fc such that when administered to the patient in combination with the switch ligand it is not rapidly cleared from the circulation but rather binds and accumulates at the site of the tumor without recruiting T cells until such time as when the switch ligand has pharmacokinetically cleared from the system thus increasing binding affinity for the target (e.g., CD3) and thereby recruiting and activating T-cells effector function.

### Methods of Use, Formulation, Administration, and Treatment

The bispecific molecules provided herein have use in redirecting immune effector cells to a target cell of interest and can be administered to a patient for treatment of a disease or condition. In various embodiments, the disclosure provides T-cell redirecting bispecific molecules. As a skilled artisan would appreciate, the provided bispecific molecules can be used to treat any disease or condition that may be treated by redirecting an immune effector cell response, such as a T-cell mediated immune response to a target cell containing a targetable cell surface antigen. Exemplary conditions that can be treated with the provided bispecific molecules include, but are not limited to, cancer, hyperplasia, neurodegenerative disease, Alzheimer's disease, cardiovascular disease, metabolic disease, vasculitis, viral infection, fungal infection, bacterial infection, diabetic retinopathy, macular degeneration, autoimmune disease, edema, pulmonary hypertension, sepsis, myocardial angiogenesis, plaque neovascularization, restenosis, neointima formation after vascular trauma, telangiectasia, hemophiliac joints, angiofibroma, fibrosis associated with chronic inflammation, lung fibrosis, deep venous thrombosis and wound granulation.

In some embodiments, a provided bispecific molecule is used to treat cancer, an inflammatory disease, an autoimmune disease or an infectious disease.

In some embodiments, the present disclosure is directed to antibody-based therapies which involve administering a bispecific molecule of the disclosure to a patient such as an animal, a mammal, and a human, for treating one or more of the disorders or conditions described herein. Therapeutic compounds of the disclosure include, but are not limited to, bispecific molecules (including variants and derivatives thereof as described herein), nucleic acids or polynucleotides encoding these bispecific molecules (including variants and derivatives thereof), and pharmaceutical compositions containing the provided bispecific molecules and nucleic acids.

In some embodiments, a provided bispecific molecule is used to treat, inhibit or prevent a disease, disorder or condition associated with increased cell survival, or the inhibition of apoptosis, for example cancer, autoimmune disorders, pathogen associated infections (e.g., viral infections such as herpes viruses, pox viruses and adenoviruses), an inflammatory condition, graft vs. host disease (acute and/or chronic), acute graft rejection, and chronic graft rejection.

Provided bispecific molecules have use in inhibiting growth, progression, and/or metastasis of cancer. In some embodiments, the provided bispecific molecules have use in treating cancer. In some embodiments, the disclosure provides a method of treating cancer that comprises administering an effective amount of a bispecific molecule provided herein to a patient in need thereof. In some embodiments, the cancer treated according to the provided method is a hematological cancer or a solid tumor.

The term “effective amount” used herein refers to an amount of a compound or composition sufficient to treat a specified disorder, condition or disease such as to ameliorate, palliate, lessen, and/or delay one or more of its symptoms. In reference to cancer, an effective amount comprises an amount sufficient to kill a tumor cell or to cause a tumor to shrink and/or to decrease the growth rate of the tumor (such as to suppress tumor growth) or to prevent or delay other unwanted cancer cell proliferation In some embodiments, an effective amount is an amount sufficient to delay development of a disease or condition. In some embodiments, an effective amount is an amount sufficient to prevent or delay recurrence of a disease or condition. An effective amount can be administered in one or more administrations.

In some embodiments, the cancer treated according to the provided method is a hematological cancer. In some embodiments, the hematological cancer treated according to the provided methods is selected from: Non-Hodgkin Lymphoma, multiple myeloma, acute myeloid leukemia, Hodgkin's lymphoma, acute lymphoblastic leukemia, chronic lymphocytic leukemia, myelodysplastic syndromes, follicular lymphoma, chronic myelogenous leukemia, diffuse large B-cell lymphoma, cutaneous T-cell lymphoma, acute eosinophilic leukemia, anaplastic large cell lymphoma, Burkitt's lymphoma, and angioimmunoblastic T-cell lymphoma.

In some embodiments, the cancer treated according to the provided method is a solid tumor. In some embodiments, the solid tumor treated according to the provided methods is selected from: prostate cancer, breast cancer, lung cancer (non-small cell and small cell), colon cancer, rectal cancer, bladder cancer, pancreatic cancer, stomach (gastric) cancer, liver (hepatocellular) cancer, endometrial cancer, ovarian cancer, cervical cancer, esophageal cancer, head and neck cancer, oral cancer, melanoma, thyroid cancer, kidney (renal cell) cancer, testicular cancer, penile cancer, Anal cancer, retinoblastoma, uveal melanoma, merkel cell carcinoma, chondrosarcoma, Ewing's sarcoma, osteosarcoma, astrocytoma, glioblastoma, neuroblastoma, and mesothelioma.

In some embodiments, the cancer treated according to the provided methods is selected from: acute lymphoblastic leukemia, acute myelogenous leukemia, Hodgkin's lymphoma, non-Hodgkin's lymphoma, lymphocytic leukemia, chronic myelogenous leukemia, multiple myeloma, Kaposi's sarcoma, biliary cancer, cervical cancer, breast cancer, chronic colorectal cancer, endometrial cancer, colon cancer, esophageal cancer, gastric cancer, intestinal cancer, head and neck cancer, lung cancer, medullary thyroid cancer, retinoblastoma, renal cancer, pancreatic cancer, glioma, melanoma, osteoblastoma, osteoclastoma, glioblastoma, osteosarcoma, liver cancer, prostate cancer, ovarian cancer, testicular cancer, a cardiac tumor, stomach cancer, neuroblastoma, myxoma, myoma, endothelioma, chondrosarcoma, adenoma, and urinary bladder cancer. However, the skilled artisan will realize that tumor-associated antigens are known for virtually any type of cancer.

In some embodiments, the cancer treated according to the provided methods is selected from: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, an Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer

In some embodiments the provided bispecific molecules have use in treating a disorder of the immune system such as an inflammatory disease or an autoimmune disease. In some embodiments, the disclosure provides a method of treating a disorder of the immune system that comprises administering an effective amount of a bispecific molecule provided herein to a patient in need thereof. In some embodiments the provided bispecific molecules have use in treating an autoimmune disease. In some embodiments, the disclosure provides a method of treating an autoimmune disease that comprises administering an effective amount of a bispecific molecule provided herein to a patient in need thereof. In some embodiments, the autoimmune disease treated according to the methods provided herein is selected from rheumatoid arthritis, acute idiopathic thrombocytopenic purpura, chronic idiopathic thrombocytopenic purpura, dermatomyositis, Grave's disease, Behcet's disease, Crohn's disease, autoimmune gastritis, Sydenham's chorea, myasthenia gravis, systemic lupus erythematosus, lupus nephritis, rheumatic fever, polyglandular syndromes, bullous pemphigoid, type 1 diabetes, type 2 diabetes, Henoch-Schonlein purpura, post-streptococcalnephritis, erythema nodosum, Takayasu's arteritis, Addison's disease, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, immune-related glomerulonephritis, psoriasis and fibrosing alveolitis.

In some embodiments, the provided bispecific molecules have use in treating a disorder or condition associated with the infection of a pathogenic organism. The bispecific molecules can also be used to treat an infectious disease caused by a pathogenic organism, or to kill the organism, by targeting the pathogenic organism and an immune cell to effect elimination of the organism.

In some embodiments, the disclosure provides a method of treating a disorder or condition associated with the infection of a pathogenic organism that comprises administering an effective amount of a bispecific molecule provided herein to a patient in need thereof. In some embodiments, treated disorder or condition is associated with a pathogenic agent selected from an RNA virus, a DNA virus, a Gram positive bacterium, a Gram negative bacterium, a protozoa or a fungus.

In other embodiments, the subject bsAbs may be of use to treat subjects infected with a pathogenic organism, such as bacteria, viruses or fungi. Exemplary fungi that may be treated include Microsporum, Trichophyton, Epidermophyton, Sporothrix schenckii, Cryptococcus neoformans, Coccidioides immitis, Histoplasma capsulatum, Blastomyces dermatitidis or Candida albican. Exemplary viruses include human immunodeficiency virus (HIV), herpes virus, cytomegalovirus, rabies virus, influenza virus, human papilloma virus, hepatitis B virus, hepatitis C virus, Sendai virus, feline leukemia virus, Reo virus, polio virus, human serum parvo-like virus, simian virus 40, respiratory syncytial virus, mouse mammary tumor virus, Varicella-Zoster virus, dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus or blue tongue virus. Exemplary bacteria include Bacillus anthracis, Streptococcus agalactiae, Legionella pneumophilia, Streptococcus pyogenes, Escherichia coli, Neisseria gonorrhoeae, Neisseria meningitidis, Pneumococcus spp., Hemophilis influenzae B, Treponema pallidum, Lyme disease spirochetes, Pseudomonas aeruginosa, Mycobacterium leprae, Brucella abortus, Mycobacterium tuberculosis or a Mycoplasma.

In other embodiments, provide bispecific molecules have use in treating subjects infected with a pathogenic organism, characterized by the presence of at least one of: Epstein Barr virus, Hepatitis A Virus (HAV); Hepatitis B Virus (HBV); Hepatitis C Virus (HCV); SARS-CoV-2, herpes viruses (e.g., HSV-1, HSV-2, HHV-6, CMV), Human Immunodeficiency Virus (HIV), Vesicular Stomatitis Virus (VSV), Bacilli, Citrobacter, Cholera, Diphtheria, Enterobacter, Gonococci, Helicobacter pylori, Klebsiella, Legionella, Meningococci, mycobacteria, Pseudomonas, Pneumonococci, rickettsia bacteria, Salmonella, Serratia, Staphylococci, Streptococci, Tetanus, Aspergillus (A. fumigatus, A. niger, etc.), Blastomyces dermatitidis, Candida (C. albicans, C. krusei, C. glabrata, C. tropicalis, etc.), Cryptococcus neoformans, Genus Mucorales (mucor, absidia, rhizopus), Sporothrix schenkii, Paracoccidioides brasiliensis, Coccidioides immitis, Histoplasma capsulatum, Leptospirosis, Borrelia burgdorferi, helminth parasite (hookworm, tapeworms, flukes, flatworms (e.g., Schistosomia), Giardia lambia, trichinella, Dientamoeba Fragilis, Trypanosoma brucei, Trypanosoma cruzi, or Leishmania donovani.

Methods of administration of the antigen-binding polypeptides, variants or include but are not limited to intradermal, intramuscular, intraperitoneal, intravenous, subcutaneous, intranasal, epidural, and oral routes.

In some embodiments, the bispecific molecule containing switchable scFv has been engineered for long pharmacokinetic half-life and the switch ligand has been formulated for long duration controlled release.

In some embodiments, the bispecific molecule containing switchable scFv has been engineered for long pharmacokinetic half-life and the switch ligand has not been formulated for long duration controlled release.

In some embodiments, the bispecific molecule containing switchable scFv has been engineered for short pharmacokinetic half-life and the switch ligand has been formulated for long duration controlled release.

In some embodiments, the bispecific molecule containing switchable scFv has been engineered for short pharmacokinetic half-life and the switch ligand has not been formulated for long duration controlled release.

In some embodiments the bispecific molecule containing switchable scFv is administered as a DNA or RNA based gene therapy and its biological effect is regulated by administration of a switch ligand.

In some embodiments the bispecific molecule containing switchable scFv is engineered to be secreted by an adoptive cell therapy and its biological effect is regulated by administration of a switch ligand.

In some embodiments the bispecific molecule is engineered to be delivered as part of an oncolytic virus and its biological effect is regulated by administration of a switch ligand.

In some embodiments, the switch ligand is formulated for oral administration.

Articles of manufacture, including kits containing the bispecific molecules and/or calmodulin binding ligand compositions, are provided herein. The article of manufacture may comprise a container and a label or package insert on or associated with the container. Suitable containers include, for example, bottles, vials or syringes. The containers may be formed from a variety of materials such as glass or plastic. The container holds one or more bispecific molecules and/or calmodulin binding ligand, and/or vectors or host cells of the present disclosure. The label or package insert may include directions for administering the bispecific molecules and/or calmodulin binding ligand compositions to a patient. Such kits have uses including, but not limited to, therapeutic applications of the bispecific molecules and/or calmodulin binding ligand compositions.

All publications, patents, patent applications, internet sites, and accession numbers/database sequences (including both polynucleotide and polypeptide sequences) cited are herein incorporated by reference in their entirety for all purposes to the same extent as if each individual publication, patent, patent application, internet site, or accession number/database sequence were specifically and individually indicated to be so incorporated by reference.

Various embodiments of the invention will now be illustrated through the description of experiments conducted in accordance therewith. The examples that follow are provided to facilitate the practice of the disclosed embodiments, and are not to be construed as limiting in any way the remainder of the disclosure. In the examples, reference is made to the appended figures.

### EXAMPLES

### Example 1: Description of Bi-Specific Anti-CD33-CD3 Antigen-Binding Constructs

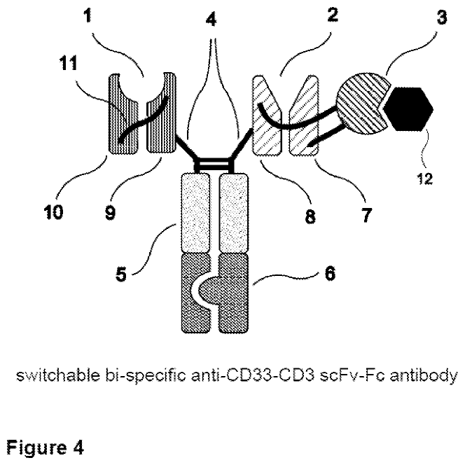

A number of exemplary bi-specific anti-CD33-CD3 antigen-binding constructs are designed as described below. An exemplary schematic representation of this type of construct is shown in FIG. 4. All depicted formats are based on a heterodimeric “knob into hole” Fc that is constructed by mutations in the CH3 domain (see, e.g., John et al., r, ‘Knobs-into-holes’ engineering of antibody CH3 domains for heavy chain heterodimerization, Protein Engineering, Design and Selection 9 (7):617-622 (1996); doi.org/10.1093/protein/9.7.617, Von Kreudenstein et al, MAbs. 2013 5(5):646-54). This technology allows to generate bispecific molecules that contain the heterodimeric Fc with an anti-CD33 scFv and anti-CD3 scFv (FIG. 4) or respective Fab/scFc Fc molecules contain the heterodimeric Fc with an anti-CD33 Fab and anti-CD3 Fab (FIG. 2).

### Example 2: Generation of DNA Encoding a Switchable Bispecific Antibody

The anti-CD33 switchable T cell engager is generated by fusing the OKT3 (Orthoclone OKT3, muronomab) variable heavy chain sequence (SEQ ID NO:86) to the OKT3 variable light chain sequences (SEQ ID NO:87) with a switchable linker (one of the SEQ ID NO: 92 to EQ ID NO: 278) connecting them. The cloning is performed as described (Kellmann S J, Dubel S, Thie H. A strategy to identify linker-based modules for the allosteric regulation of antibody-antigen binding affinities of different scFvs. MAbs. 2017 9(3):404-418. doi: 10.1080/19420862.2016.) An anti-CD33 scFv is constructed from the variable heavy chain sequence of gemtuzumab (SEQ ID NO:351) to the gemtuzumab variable light chain sequence (SEQ ID NO:352) with a (GGGGS)3 linker (SEQ ID NO:365) between the light and heavy chain. The DNA encoding the switchable anti-CD3 scFv as well as the anti CD33scFv is then fused to the hinge region of two different DNA expression vectors, respectively, that together can form an heterologous Fc part, the two chains of which have been modified for the preferred formation of bispecific antibodies by “knob-into-hole” mutations within the CH3 domain as described in U.S. Pat. No. 8,216,805B2. This method involves introducing a protuberance at the interface of one of the Fc parts and a corresponding cavity in the interface of the second Fc parts, such that the protuberance can be positioned in the cavity so as to promote heterodimer formation and hinder homodimer formation of the CH3 chains.

Specifically, for the production of product MS42-13, the mutation T366Y is introduced into the CH3 region of the anti-CD3 Fc fragment whereas the anti-CD33 Fc fragment fused to carried the mutations Y407T in the CH3 region. The complete expression cassettes encoding each scFv-Fc fusion DNA are cloned into expression vector pCSE2.5 for transient expression in mammalian cells (Schirrmann et al., MAbs 2:73-6 (2010); doi.org/10.4161/mabs.2.1.10784).

Vector constructs encoding the anti-CD3 scFv-Fc-Fusion are derived from OKT3 with different switch linkers from the list of SEQ ID NO:92-SEQ ID NO:287 are co-transfected into EXPI293 cells together with a vector construct comprising the insert encoding the anti CD33 scFv-Fc-Fusion derived from gemtuzumab as described in SEQ ID NO:366 in different ratios of the DNA of the two combined vectors. Vector ratios of the vector encoding the anti-CD33 construct versus the vector encoding the CD3 construct of 3.4:1, 1:1, 1:2, 1:3.4 and 1:5.2 are examined for maximal yield of protein per cell/day as determined after protein A affinity purification.

```
SEQ ID NO: 366:
DNA insert encoding an anti CD33 scFv-Fc-Fusion for bispecific antibody
generation
    1-57 Signal Sequence
  58-405 VH of Gemtuzumab
406-450 GlySer-Linker
451-783 VL of Gemtuzumab
784-819 Hinge
820-1479 CH2/CH3 ″hole″
The mature product after cleaving off of the signal peptide is encoded by
nucleotides 58-1479 of SEQ ID NO: 366.
(SEQ ID NO: 366)
ATGACCCGGCTGACAGTCCTGGCCCTGCTGGCTGGTCTGCTGGCGTCCTCGAGGGCCGAGGTGCAGCTGG
TGCAGAGCGGCGCCGAGGTGAAGAAGCCCGGCAGCAGCGTGAAGGTGAGCTGCAAGGCCAGCGGCTACA
CCATCACCGACAGCAACATCCACTGGGTGCGCCAGGCCCCCGGCCAGAGCCTGGAGTGGATCGGCTACATC
TACCCCTACAACGGCGGCACCGACTACAACCAGAAGTTCAAGAACCGCGCCACCCTGACCGTGGACAACCC
CACCAACACCGCCTACATGGAGCTGAGCAGCCTGCGCAGCGAGGACACCGCCTTCTACTACTGCGTGAACG
GCAACCCCTGGCTGGCCTACTGGGGCCAGGGCACCCTGGTGACCGTGAGCAGCGGTGGAGGCGGTTCAG
GCGGAGGTGGCAGCGGCGGTGGCGGGTCGGACATCCAGCTGACCCAGAGCCCCAGCACCCTGAGCGCCA
GCGTGGGCGACCGCGTGACCATCACCTGCCGCGCCAGCGAGAGCCTGGACAACTACGGCATCCGCTTCCT
GACCTGGTTCCAGCAGAAGCCCGGCAAGGCCCCCAAGCTGCTGATGTACGCCGCCAGCAACCAGGGCAGC
GGCGTGCCCAGCCGCTTCAGCGGCAGCGGCAGCGGCACCGAGTTCACCCTGACCATCAGCAGCCTGCAGC
CCGACGACTTCGCCACCTACTACTGCCAGCAGACCAAGGAGGTGCCCTGGAGCTTCGGCCAGGGCACCAA
GGTGGAGGTGAAGGAGCCCAAGAGCTGCGACAAGACCCACACCTGCCCCCCCTGCCCCGCCCCCGAGCTG
CTGGGCGGCCCCAGCGTGTTCCTGTTCCCCCCCAAGCCCAAGGACACCCTGATGATCAGCCGCACCCCCGA
GGTGACCTGCGTGGTGGTGGACGTGAGCCACGAGGACCCCGAGGTGAAGTTCAACTGGTACGTGGACGG
CGTGGAGGTGCACAACGCCAAGACCAAGCCCCGCGAGGAGCAGTACAACAGCACCTACCGCGTGGTGAGC
GTGCTGACCGTGCTGCACCAGGACTGGCTGAACGGCAAGGAGTACAAGTGCAAGGTGAGCAACAAGGCC
CTGCCCGCCCCCATCGAGAAGACCATCAGCAAGGCCAAGGGCCAGCCCCGCGAGCCCCAGGTGTACACCCT
GCCCCCCAGCCGCGACGAGCTGACCAAGAACCAGGTGAGCCTGACCTGCCTGGTGAAGGGCTTCTACCCCA
GCGACATCGCCGTGGAGTGGGAGAGCAACGGCCAGCCCGAGAACAACTACAAGACCACCCCCCCCGTGCT
GGACAGCGACGGCAGCTTCTTCCTGACCAGCAAGCTGACCGTGGACAAGAGCCGCTGGCAGCAGGGCAAC
GTGTTCAGCTGCAGCGTGATGCACGAGGCCCTGCACAACCACTACACCCAGAAGAGCCTGAGCCTGAGCCC
CGGCAAG
SEQ ID NO: 367:
DNA insert encoding an anti CD3 scFv-Fc-Fusion with switch linker SEQ ID
NO: 119
      1-57 Signal Sequence,
    58-426 OKT 3 VH region
  427-828 switch linker
  829-1155 OKT 3 VL region
1156-1191 Hinge
1192-1851 CH2/CH3 ″knob″
The mature product after cleaving off of the signal peptide is encoded by
nucleotides 58-1851 of SEQ ID NO: 367.
(SEQ ID NO: 367)
ATGACCCGGCTGACAGTCCTGGCCCTGCTGGCTGGTCTGCTGGCGTCCTCGAGGGCCGAGGTGCAG
CTGGTGGAGAGCGGCGGCGGCCTGGTGCAGCCCGGCCGCAGCCTGCGCCTGAGCTGCGCCGCCAGC
GGCTTCACCTTCGACGACTACAGCATGCACTGGGTGCGCCAGGCCCCCGGCAAGGGCCTGGAGTGG
GIGAGCGGCATCAGCTGGAACAGCGGCAGCATCGGCTACGCCGACAGCGTGAAGGGCCGCTTCACC
ATCAGCCGCGACAACGCCAAGAACAGCCTGTACCTGCAGATGAACAGCCTGCGCGCCGAGGACACC
GCCCTGTACTACTGCGCCAAGTACGGCAGCGGCTACGGCAAGTTCTACTACTACGGCATGGACGTG
TGGGGCCAGGGCACCACCGTGACCGTGAGCGCCGAGTTCAAGGAGGCCTTCAGCCTGTTCGACAAG
GACGGCGACGGCACCATCACCACCAAGGAGCTGGGCACCGTGATGCGCAGCCTGGGCCAGAACCCC
ACCGAGGCCGAGCTGCAGGACATGATCAACGAGGTGGACGCCGACGGCAACGGCACCATCGACTTC
CCCGAGTTCCTGACCATGATGGCCCGCAAGATGAAGGACACCGACAGCGAGGAGGAGATCCGCGAG
GCCTTCCGCGTGTTCGACAAGGACGGCAACGGCTACATCAGCGCCGCCGAGCTGCGCCACGTGATG
ACCAACCTGGGCGAGAAGCTGACCGACGAGGAGGTGGACGAGATGATCCGCGAGGCCGACATCGAC
GGCGACGGCCAGGTGAACTACGAGGAGTTCGTGAAGCAGATCGTGCTGACCCAGAGCCCCGCCATC
ATGAGCGCCAGCCCCGGCGAGAAGGIGACCATGACCTGCAGCGCCAGCAGCAGCGTGAGCTACATG
AACTGGTACCAGCAGAAGAGCGGCACCAGCCCCAAGCGCTGGATCTACGACACCAGCAAGCTGGCC
AGCGGCGTGCCCGCCCACTTCCGCGGCAGCGGCAGCGGCACCAGCTACAGCCTGACCATCAGCGGC
ATGGAGGCCGAGGACGCCGCCACCTACTACTGCCAGCAGTGGAGCAGCAACCCCTTCACCTTCGGC
AGCGGCACCAAGCTGGAGATCAACCGCGCCGACGAGCCCAAGAGCTGCGACAAGACCCACACCIGC
CCCCCCTGCCCCGCCCCCGAGCTGCTGGGCGGCCCCAGCGTGTTCCTGTTCCCCCCCAAGCCCAAG
GACACCCTGATGATCAGCCGCACCCCCGAGGIGACCTGCGTGGTGGTGGACGTGAGCCACGAGGAC
CCCGAGGIGAAGTTCAACTGGIACGTGGACGGCGTGGAGGTGCACAACGCCAAGACCAAGCCCCGC
GAGGAGCAGTACAACAGCACCTACCGCGTGGTGAGCGTGCTGACCGTGCTGCACCAGGACTGGCTG
AACGGCAAGGAGTACAAGTGCAAGGTGAGCAACAAGGCCCTGCCCGCCCCCATCGAGAAGACCATC
AGCAAGGCCAAGGGCCAGCCCCGCGAGCCCCAGGIGTACACCCTGCCCCCCAGCCGCGACGAGCTG
ACCAAGAACCAGGTGAGCCTGTACTGCCTGGTGAAGGGCTTCTACCCCAGCGACATCGCCGIGGAG
TGGGAGAGCAACGGCCAGCCCGAGAACAACTACAAGACCACCCCCCCCGTGCTGGACAGCGACGGC
AGCTTCTTCCTGTACAGCAAGCTGACCGTGGACAAGAGCCGCTGGCAGCAGGGCAACGIGTTCAGC
TGCAGCGTGATGCACGAGGCCCTGCACAACCACTACACCCAGAAGAGCCTGAGCCTGAGCCCCGGC
AAG
SEQ ID NO: 368:
DNA insert encoding an anti CD3 scFv-Fc-Fusion with switch linker SEQ ID NO:
110.The mature product after cleaving off of the signal peptide is encoded by nucleotides
58-1851 of SEQ ID NO: 368.
(SEQ ID NO: 368)
ATGACCCGGCTGACAGTCCTGGCCCTGCTGGCTGGTCTGCTGGCGTCCTCGAGGGCCGAGGTGCAG
CTGGTGGAGAGCGGCGGCGGCCTGGTGCAGCCCGGCCGCAGCCTGCGCCTGAGCTGCGCCGCCAGC
GGCTTCACCITCGACGACTACAGCATGCACTGGGTGCGCCAGGCCCCCGGCAAGGGCCTGGAGTGG
GTGAGCGGCATCAGCTGGAACAGCGGCAGCATCGGCTACGCCGACAGCGTGAAGGGCCGCTTCACC
ATCAGCCGCGACAACGCCAAGAACAGCCTGTACCTGCAGATGAACAGCCTGCGCGCCGAGGACACC
GCCCTGTACTACTGCGCCAAGTACGGCAGCGGCTACGGCAAGTTCTACTACTACGGCATGGACGTG
TGGGGCCAGGGCACCACCGTGACCGTGAGCGCCGAGTTCAAGGAGGCCTTCAGCCTGTTCGACAAG
GACGGCGACGGCACCATCACCACCAAGGAGCTGGGCACCGTGATGCGCAGCCTGGGCCAGAACCCC
ACCGAGGCCGAGCTGCAGGACATGATCAACGAGGTGGACGCCGACGGCAACGGCACCATCGACTTC
CCCGAGTTCCTGACCATGATGGCCCGCAAGATGAAGGACACCGACAGCGAGGAGGAGATCCGCGAG
GCCTTCCGCGTGTTCGACAAGGACGGCAACGGCTACATCAGCGCCGCCGAGCTGCGCCACGTGATG
ACCAACCTGGGCGAGAAGCTGACCGACGAGGAGGTGGACGAGATGATCCGCGAGGCCGACATCGAC
GGCGACGGCCAGGTGAACTACGAGGAGTTCGTGCAGCAGATCGTGCTGACCCAGAGCCCCGCCATC
ATGAGCGCCAGCCCCGGCGAGAAGGTGACCATGACCTGCAGCGCCAGCAGCAGCGTGAGCTACATG
AACTGGTACCAGCAGAAGAGCGGCACCAGCCCCAAGCGCTGGATCTACGACACCAGCAAGCTGGCC
AGCGGCGTGCCCGCCCACTTCCGCGGCAGCGGCAGCGGCACCAGCTACAGCCTGACCATCAGCGGC
ATGGAGGCCGAGGACGCCGCCACCTACTACTGCCAGCAGTGGAGCAGCAACCCCTTCACCTTCGGC
AGCGGCACCAAGCTGGAGATCAACCGCGCCGACGAGCCCAAGAGCTGCGACAAGACCCACACCTGC
CCCCCCTGCCCCGCCCCCGAGCTGCTGGGCGGCCCCAGCGTGTTCCTGTTCCCCCCCAAGCCCAAG
GACACCCTGATGATCAGCCGCACCCCCGAGGTGACCTGCGTGGTGGTGGACGTGAGCCACGAGGAC
CCCGAGGTGAAGTTCAACTGGTACGTGGACGGCGTGGAGGTGCACAACGCCAAGACCAAGCCCCGC
GAGGAGCAGTACAACAGCACCTACCGCGTGGTGAGCGTGCTGACCGTGCTGCACCAGGACTGGCTG
AACGGCAAGGAGTACAAGTGCAAGGTGAGCAACAAGGCCCTGCCCGCCCCCATCGAGAAGACCATC
AGCAAGGCCAAGGGCCAGCCCCGCGAGCCCCAGGTGTACACCCTGCCCCCCAGCCGCGACGAGCTG
ACCAAGAACCAGGTGAGCCTGTACTGCCTGGTGAAGGGCTTCTACCCCAGCGACATCGCCGTGGAG
TGGGAGAGCAACGGCCAGCCCGAGAACAACTACAAGACCACCCCCCCCGTGCTGGACAGCGACGGC
AGCTTCTTCCTGTACAGCAAGCTGACCGTGGACAAGAGCCGCTGGCAGCAGGGCAACGTGTTCAGC
TGCAGCGTGATGCACGAGGCCCTGCACAACCACTACACCCAGAAGAGCCTGAGCCTGAGCCCCGGC
AAG
SEQ ID NO: 369:
DNA insert encoding an anti CD3 scFv-Fc-Fusion with switch linker SEQ ID NO:
121. The mature product after cleaving off of the signal peptide is encoded by nucleotides
58-1821 of SEQ ID NO: 369.
(SEQ ID NO: 369)
ATGACCCGGCTGACAGTCCTGGCCCTGCTGGCTGGTCTGCTGGCGTCCTCGAGGGCCGAGGTGCAG
CTGGTGGAGAGCGGCGGCGGCCTGGTGCAGCCCGGCCGCAGCCTGCGCCTGAGCTGCGCCGCCAGC
GGCTTCACCTTCGACGACTACAGCATGCACTGGGTGCGCCAGGCCCCCGGCAAGGGCCTGGAGTGG
GTGAGCGGCATCAGCTGGAACAGCGGCAGCATCGGCTACGCCGACAGCGTGAAGGGCCGCTTCACC
ATCAGCCGCGACAACGCCAAGAACAGCCTGTACCTGCAGATGAACAGCCTGCGCGCCGAGGACACC
GCCCTGTACTACTGCGCCAAGTACGGCAGCGGCTACGGCAAGTICTACTACTACGGCATGGACGTG
TGGGGCCAGGGCACCACCGTGACCGTGAGCGACAAGGACGGCGACGGCACCATCACCACCAAGGAG
CTGGGCACCGTGATGCGCAGCCTGGGCCAGAACCCCACCGAGGCCGAGCTGCAGGACATGATCAAC
GAGGTGGACGCCGACGGCAACGGCACCATCGACTTCCCCGAGTTCCTGACCATGATGGCCCGCAAG
ATGAAGGACACCGACAGCGAGGAGGAGATCCGCGAGGCCTTCCGCGTGTTCGACAAGGACGGCAAC
GGCTACATCAGCGCCGCCGAGCTGCGCCACGTGATGACCAACCTGGGCGAGAAGCTGACCGACGAG
GAGGTGGACGAGATGATCCGCGAGGCCGACATCGACGGCGACGGCCAGGTGAACTACGAGGAGTTC
GTGAAGCAGATCGTGCTGACCCAGAGCCCCGCCATCATGAGCGCCAGCCCCGGCGAGAAGGTGACC
ATGACCTGCAGCGCCAGCAGCAGCGTGAGCTACATGAACTGGTACCAGCAGAAGAGCGGCACCAGC
CCCAAGCGCTGGATCTACGACACCAGCAAGCTGGCCAGCGGCGTGCCCGCCCACTTCCGCGGCAGC
GGCAGCGGCACCAGCTACAGCCTGACCATCAGCGGCATGGAGGCCGAGGACGCCGCCACCTACTAC
TGCCAGCAGTGGAGCAGCAACCCCTTCACCTTCGGCAGCGGCACCAAGCTGGAGATCAACCGCGCC
GACGAGCCCAAGAGCTGCGACAAGACCCACACCTGCCCCCCCTGCCCCGCCCCCGAGCTGCTGGGC
GGCCCCAGCGTGTTCCTGTTCCCCCCCAAGCCCAAGGACACCCTGATGATCAGCCGCACCCCCGAG
GTGACCTGCGTGGTGGTGGACGTGAGCCACGAGGACCCCGAGGTGAAGTTCAACTGGIACGTGGAC
GGCGTGGAGGIGCACAACGCCAAGACCAAGCCCCGCGAGGAGCAGTACAACAGCACCTACCGCGTG
GTGAGCGTGCTGACCGTGCTGCACCAGGACTGGCTGAACGGCAAGGAGTACAAGTGCAAGGTGAGC
AACAAGGCCCTGCCCGCCCCCATCGAGAAGACCATCAGCAAGGCCAAGGGCCAGCCCCGCGAGCCC
CAGGTGTACACCCTGCCCCCCAGCCGCGACGAGCTGACCAAGAACCAGGTGAGCCTGTACTGCCTG
GTGAAGGGCTTCTACCCCAGCGACATCGCCGTGGAGTGGGAGAGCAACGGCCAGCCCGAGAACAAC
TACAAGACCACCCCCCCCGTGCTGGACAGCGACGGCAGCTTCTTCCTGTACAGCAAGCTGACCGTG
GACAAGAGCCGCTGGCAGCAGGGCAACGTGTTCAGCTGCAGCGTGATGCACGAGGCCCTGCACAAC
CACTACACCCAGAAGAGCCTGAGCCTGAGCCCCGGCAAG
SEQ ID NO: 370:
DNA insert encoding an anti CD3 scFv-Fc-Fusion with switch linker SEQ ID NO:
227. The mature product after cleaving off of the signal peptide is encoded by nucleotides
58-1860 of SEQ ID NO: 370.
(SEQ ID NO: 370)
ATGACCCGGCTGACAGTCCTGGCCCTGCTGGCTGGTCTGCTGGCGTCCTCGAGGGCCGAGGTGCAG
CTGGTGGAGAGCGGCGGCGGCCTGGTGCAGCCCGGCCGCAGCCTGCGCCTGAGCTGCGCCGCCAGC
GGCTTCACCTTCGACGACTACAGCATGCACTGGGTGCGCCAGGCCCCCGGCAAGGGCCTGGAGTGG
GTGAGCGGCATCAGCTGGAACAGCGGCAGCATCGGCTACGCCGACAGCGTGAAGGGCCGCTTCACC
ATCAGCCGCGACAACGCCAAGAACAGCCTGTACCTGCAGATGAACAGCCTGCGCGCCGAGGACACC
GCCCTGTACTACTGCGCCAAGTACGGCAGCGGCTACGGCAAGTTCTACTACTACGGCATGGACGTG
TGGGGCCAGGGCACCACCGTGACCGTGAGCGCCGAGTTCAAGGAGGCCTTCAGCCTGTTCGACAAG
GACGGCGACGGCACCATCACCACCAAGGAGCTGGGCACCGIGATGCGCAGCCTGGGCCAGAACCCC
ACCGAGGCCGAGCTGCAGGACATGATCAACGAGGTGGACGCCGACGGCAACGGCACCATCGACTTC
CCCGAGTTCCTGACCATGATGGCCCGCAAGATGAAGGACACCGACAGCGAGGAGGAGATCCGCGAG
GCCTTCCGCGTGTTCGACAAGGACGGCAACGGCTACATCAGCGCCGCCGAGCTGCGCCACGTGATG
ACCAACCTGGGCGAGAAGCTGACCGACGAGGAGGTGGACGAGATGATCCGCGAGGCCGACATCGAC
GGCGACGGCCAGGIGAACTACGAGGAGTTCGTGCAGATGAGCGGCCAGATCGIGCTGACCCAGAGC
CCCGCCATCATGAGCGCCAGCCCCGGCGAGAAGGTGACCATGACCTGCAGCGCCAGCAGCAGCGTG
AGCTACATGAACTGGTACCAGCAGAAGAGCGGCACCAGCCCCAAGCGCTGGATCTACGACACCAGC
AAGCTGGCCAGCGGCGTGCCCGCCCACTTCCGCGGCAGCGGCAGCGGCACCAGCTACAGCCTGACC
ATCAGCGGCATGGAGGCCGAGGACGCCGCCACCTACTACTGCCAGCAGTGGAGCAGCAACCCCTTC
ACCTTCGGCAGCGGCACCAAGCTGGAGATCAACCGCGCCGACGAGCCCAAGAGCTGCGACAAGACC
CACACCTGCCCCCCCTGCCCCGCCCCCGAGCTGCTGGGCGGCCCCAGCGTGTTCCTGTTCCCCCCC
AAGCCCAAGGACACCCTGATGATCAGCCGCACCCCCGAGGTGACCTGCGIGGIGGTGGACGTGAGC
CACGAGGACCCCGAGGTGAAGTTCAACTGGTACGTGGACGGCGTGGAGGTGCACAACGCCAAGACC
AAGCCCCGCGAGGAGCAGTACAACAGCACCTACCGCGIGGIGAGCGIGCTGACCGIGCTGCACCAG
GACTGGCTGAACGGCAAGGAGTACAAGTGCAAGGTGAGCAACAAGGCCCTGCCCGCCCCCATCGAG
AAGACCATCAGCAAGGCCAAGGGCCAGCCCCGCGAGCCCCAGGTGTACACCCTGCCCCCCAGCCGC
GACGAGCTGACCAAGAACCAGGTGAGCCTGTACTGCCTGGTGAAGGGCTTCTACCCCAGCGACATC
GCCGTGGAGTGGGAGAGCAACGGCCAGCCCGAGAACAACTACAAGACCACCCCCCCCGTGCTGGAC
AGCGACGGCAGCTTCTTCCTGTACAGCAAGCTGACCGIGGACAAGAGCCGCTGGCAGCAGGGCAAC
GTGTTCAGCTGCAGCGTGATGCACGAGGCCCTGCACAACCACTACACCCAGAAGAGCCTGAGCCTG
AGCCCCGGCAAG
```

The switchable M42-13 product is produced by co-transfecting vectors comprising the DNA inserts described in SEQ ID NO:366 and SEQ ID NO:367. For transfection, the vectors containing the respective DNA inserts are preferentially mixed in a ratio of DNA amount of the vector encoding the anti-CD33 construct versus the vector encoding the CD3 construct of 1:3.4.

### Example 3; Production and Purification of a Switchable Bispecific Antibody MS42-13

Antibodies are produced by transfecting a total of 6×10⁶ EXPI293F cells (Thermo Fisher Scientific) per transfection with a mixture of various amounts of the anti-CD3 and anti-CD33-Fc vectors (Jager et al., BMC Biotechnol. 13: 52 (2013)), as previously described. Transient production of antibody fragments is performed in EXPI293F cells. At the day of transfection, cell density is between 1.9-2.4×10⁶ cells/mL and viability >90%. Cells are transfected using polyethylenimine (PEI) and cultured at 37° C., 110 rpm and 5% C02 in serum-free Gibco FreeStyle F17 expression media supplemented with 8 mM Glutamine and 0.1% Pluronic F68. For the formation of DNA:PEI complexes, 1 μg DNA/mL transfection volume and 5 μg of 40 kDa PEI are first diluted separately in 5% transfection volume in supplemented F17 media. DNA and PEI is then mixed and incubated for 25 min at room temperature before adding the mixture to the cells. After 48 hours the culture volumes are doubled with HEKIONE T medium supplemented with 0.5% tryptone N1. Five days later, the cultures are harvested by centrifugation at for 5 mir at 290×g and subsequent centrifugation of the cell supernatant for 10 min at 3184×g. 10% (v/v) 5 M NaCl is then added to the supernatant prior to 0.2 μM filtration.

The supernatant of the cell cultivation is harvested as described and products are affinity purified using Staphylococcal protein A on Protein A HiTrap® FF columns and the Akta Prime fast performance liquid chromatography (FPLC) device (GE Healthcare, Munich, Germany) according to the manufacturer's protocols (binding buffer: 20 mM Na2HPO4×2H2O, pH 7.0; washing buffer: 100 mM citric acid, pH 5.0; elution buffer: 100 mM citric acid, pH 2.5). Elution fractions are immediately neutralized with the adequate amount of 2 M TrisHCl (Tris-(hydroxymethyl)-aminomethan-HCl, pH 9.0). The product is quantified by a human IgG/Fc capture ELISA in both purified and non-purified samples in comparison to the N protein SL human serum protein standard (DadeBehring, now Siemens Healthcare, Erlangen, Germany). Size Exclusion Chromatography (SEC) profiles will show a major single peak, indicating a content of heterodimeric products above 92%.

### Example 4. Bispecific Functionality

Bispecific functionality is tested in an ELISA using soluble Human CD3 epsilon&CD3 gamma Heterodimer Protein (Biorbyt Ltd, Cambridge, United Kingdom) that is captured to the ELISA plate surface and an Fc-fusion of CD33 that carried an AviTag™ (biotinylated) (BPS Bioscience, Inc., San Diego, USA) detected by HRP conjugated streptavidin (BPS Bioscience, Inc., San Diego, USA). Dilution curves of the protein A purified bispecific scFv-Fc constructs are compared. For control, a bispecific scFv-Fc construct wherein the linker between the OKT3 variable heavy chain sequence to the OKT3 variable light chain sequence is a non-switchable variant (GGGGSGGGGSGG GGS (SEQ ID NO: 365)) is constructed, produced and purified as described above.

Several constructs with lower affinity to the CD3 antigen are identified, with product MS42-13 showing a drop of 24.6 fold in EC50 compared to the construct with the non-switchable wild type Gly-Ser linker.

### Example 5: Detection of Switch Activity on Living Cells

The bispecific switchable anti-CD3/antiCD33 Fc fusion protein product MS42-13 is further tested for switch activity in the presence and absence of switch linkers on living cells using flow cytometry. For the cytofluorometry analysis, CD3 positive Jurkat cells (ATCC, Clone E6-1), cultivated in suspension culture as described by the provider, are seeded in FACS tubes and washed with 3 mL PBS supplemented with 2 mM EDTA and 2% (v/v) FCS (centrifugation for 5 min at 500×g and 4-C). Cells are incubated with 100 L of various dilutions of scFv-Fc bispecific antibody constructs (between 10 ng/mL and 3 g/mL) for 1 h on ice. Cells are washed two times with 3 mL PBS (centrifugation for 5 min at 500×g and 4-C) and are incubated with 100 μL Fc-fusion of CD33 that carried an AviTag™ (biotinylated) (BPS Bioscience, Inc., San Diego, USA) (Fc-specific) for 1 h on ice in the dark in either the presence or absence of the switch peptides (1 μM or 10 μM). Cells are washed two times with 3 mL PBS supplemented with switch peptide in case of the switch peptide samples, (centrifugation for 5 min at 500×g and 4-C) in and are then incubated with 100 μL Avidin FITC-conjugated (BioLegend, San Diego USA) for 30 min at 4-C in the presence of the same concentration of switch peptide. Cells are washed two times (centrifugation for 5 min at 500×g and 4-C) and resuspended in 500 μL PBS/2% (v/v), containing the same concentration of switch peptide and 2% (v/v) FCS. Measurement is performed with a Cytomics FC 500 (Beckman Coulter) with excitation at 488 nm and emission at 525 nm (FL1). A minimum of 10⁴ events are analyzed per sample. Analysis is performed with CXP analysis 2.2 software (Beckman Coulter, Fullerton, CA) and Mean Fluorescence Intensities (MFI) are determined using Flowjo™ software (Treestar).

The product MS42-13 is expected to show a maximum of 21.3 fold increase of half-maximal binding compared to the scFv construct which carries an anti-CD3 scFv with the non-switchable linker (SEQ ID NO:365) when incubated with the switch peptide variant as described in SEQ ID NO:2.

1. A bispecific molecule, comprising:
a) a first binding domain comprising a variable heavy chain region (VH) and a variable light chain region (VL) covalently connected by an allosteric switch linker comprising a calmodulin polypeptide sequence, and
b) a second binding domain that specifically binds an epitope different from that bound by the first binding domain;
wherein the first binding domain specifically binds an antigen on the surface of an immune effector cell in the presence of Ca+ and a calmodulin binding ligand.

2. The bispecific molecule according to claim 1, wherein the immune effector cell is a T cell, a natural killer (NK) cell, a neutrophil, or a macrophage.

3. The bispecific molecule according to claim 1, wherein the immune effector cell is a cytotoxic T lymphocyte.

4. The bispecific molecule according to claim 1, wherein the first binding domain specifically binds an antigen selected from CD3, CD28, CD137 (4-1BB), CD134 (0×40), CD27, ICOS the Vy9 chain of the γδ T cell receptor, CD16A, NKG2C, NKG2D, NKp30a, NKp30b, NKp44, NKp46, 2DS1, 3DS1, 3DS2, 3DS4, 2DL5a, DNAM1, CD161, and CD47.

5. The bispecific molecule according to claim 1, wherein the first binding domain binds CD3, e.g., CD3epsilon (CD3ε).

6. The bispecific molecule according to claim 1, wherein the first and second binding domains are connected by one or more covalent bonds such as a peptide bond or a disulfide bond.

7. The bispecific molecule according to claim 1, wherein the bispecific molecule is a TriFab, Fab-Fv, Fab-dsFv. MAb-Fv, IgG-Fv, trivalent IgG-Fv (mAb-Fv) fusion protein, ScFab-Fc-scFv2, scFab-Fc-scFv, scFv-Fab IgG/XmAb, tandem scFv, diabody, (scFv)2-Fab, Fab-Fc, ScFv-Fc, DVD-Ig, IgG(H)-scFv or scFv-(H)IgG, IgG(L)-scFv or scFv-(L)IgG, 2scFv-IgG or IgG-2scFv. BITE, BITE-Fc, DART, DART-Fc Tetravalent DART, TandAb, scFv-scFv-scFv or an scFv-HSA-scFv.

8. The bispecific molecule according to claim 1, wherein the first binding domain comprises a switch linker and a VH and VL comprising:
a) an HCDR1 having the amino acid sequence of SEQ ID NO:301, an HCDR2 having the amino acid sequence of SEQ ID NO:302, an HCDR3 having the amino acid sequence of SEQ ID NO:303, an LCDR1 having the having the amino acid sequence of SEQ ID NO:304, an LCDR2 having the amino acid sequence of SEQ ID NO:305, and an LCDR3 having the amino acid sequence of SEQ ID NO:306;
b) an HCDR1 having the amino acid sequence of SEQ ID NO:307, an HCDR2 having the amino acid sequence of SEQ ID NO:308, an HCDR3 having the amino acid sequence of SEQ ID NO:309, an LCDR1 having the having the amino acid sequence of SEQ ID NO:310, an LCDR2 having the amino acid sequence of SEQ ID NO:311, and an LCDR3 having the amino acid sequence of SEQ ID NO:312;
c) an HCDR1 having the amino acid sequence of SEQ ID NO:313, an HCDR2 having the amino acid sequence of SEQ ID NO:314, an HCDR3 having the amino acid sequence of SEQ ID NO:315, an LCDR1 having the having the amino acid sequence of SEQ ID NO:316, an LCDR2 having the amino acid sequence of SEQ ID NO:317, and an LCDR3 having the amino acid sequence of SEQ ID NO:318;
d) an HCDR1 having the amino acid sequence of SEQ ID NO:319, an HCDR2 having the amino acid sequence of SEQ ID NO:320, an HCDR3 having the amino acid sequence of SEQ ID NO:321, an LCDR1 having the having the amino acid sequence of SEQ ID NO:322, an LCDR2 having the amino acid sequence of SEQ ID NO:323, and an LCDR3 having the amino acid sequence of SEQ ID NO:324;
e) an HCDR1 having the amino acid sequence of SEQ ID NO:325, an HCDR2 having the amino acid sequence of SEQ ID NO:326, an HCDR3 having the amino acid sequence of SEQ ID NO:327, an LCDR1 having the having the amino acid sequence of SEQ ID NO:328, an LCDR2 having the amino acid sequence of SEQ ID NO:329, and an LCDR3 having the amino acid sequence of SEQ ID NO:330; or
f) an HCDR1 having-the amino acid sequence of SEQ ID NO:331, an HCDR2 having the amino acid sequence of SEQ ID NO:332, an HCDR3 having the amino acid sequence of SEQ ID NO:333, an LCDR1 having the having the amino acid sequence of SEQ ID NO:334, an LCDR2 having the amino acid sequence of SEQ ID NO:335, and an LCDR3 having the amino acid sequence of SEQ ID NO:336;
and the first binding domain specifically binds CD3 on the surface of an immune effector cell in the presence of Ca+ and a calmodulin binding ligand.

9. The bispecific molecule according to claim 1, wherein the first binding domain comprises a switch linker and a VH and VL comprising:
a) a VH having the amino acid sequence of SEQ ID NO:86 and a VL having the amino acid sequence of SEQ ID NO:87, or a humanized version thereof,
b) a VH having the amino acid sequence of SEQ ID NO:88 and a VL having the amino acid sequence of SEQ ID NO:89, or a humanized version thereof, and
c) a VH having the amino acid sequence of SEQ ID NO:90 and a VL having the amino acid sequence of SEQ ID NO:91, or a humanized version thereof,
d) a VH having the amino acid sequence of SEQ ID NO:288 and a VL having the amino acid sequence of SEQ ID NO:289, or a humanized version thereof,
e) a VH having the amino acid sequence of SEQ ID NO:290 and a VL having the amino acid sequence of SEQ ID NO:291, or a humanized version thereof,
f) a VH having the amino acid sequence of SEQ ID NO:292 and a VL having the amino acid sequence of SEQ ID NO:293, or a humanized version thereof,
g) a VH having the amino acid sequence of SEQ ID NO:294 and a VL having the amino acid sequence of SEQ ID NO:295, or a humanized version thereof, or
h) a VH having the amino acid sequence of SEQ ID NO:296 or 297 and a VL having the amino acid sequence of SEQ ID NO:298, 299, or 300;
and the first binding domain specifically binds CD3 on the surface of an immune effector cell in the presence of Ca+ and a calmodulin binding ligand.

10. (canceled)

11. (canceled)

12. (canceled)

13. (canceled)

14. (canceled)

15. The bispecific molecule according to claim 1, wherein the second binding domain comprises a VH domain of an immunoglobulin and a VL domain of an immunoglobulin that is also covalently connected by an allosteric switch linker containing a calmodulin polypeptide sequence that specifically binds the epitope in the presence of Ca-i- and a calmodulin binding ligand.

16. The bispecific molecule according to claim 1, wherein the second binding domain specifically binds a cell surface antigen selected from CD 19, CD20, CD33, CD123, FLT3, CLL1, WT1, BCMA, GPRC5D, FcRH5, PSMA, DLL3, MUC1, MUC16, MUC17, EGFRviii, Mesothelin, STEAP-1, SSTR2, HER2, GPC3, GD2, TRAIL-R2, EPCAM, and CLDN18.2.

17. The bispecific molecule according to claim 1, wherein the second binding domain comprises a VH and a VL that is also covalently connected by an allosteric switch linker containing a calmodulin polypeptide sequence that specifically binds the different epitope in the presence of Ca-i- and a calmodulin binding ligand.

18. The bispecific molecule according to claim 1, wherein the allosteric switch linker comprises:
a) any one of the sequences of SEQ ID NO:92 to SEQ ID NO:287;
b) a fragment, variant, or acyclic mutant of any one of the sequences of SEQ ID NO:92 to SEQ ID NO:287;
c) a sequence having at least 75%, 80%, 85%, 90% 95%, 96%, 97%, 98%, or 99% sequence identity with at least one of the sequences of SEQ ID 92-287; or
d) a sequence that contains 1-25, 1-20, 1-15, 1-10, 1-5 or 5-20 amino acid additions, substitutions, and/or deletions compared to one at least one of the sequences of SEQ ID 92-287.

19. The bispecific molecule according to claim 1, wherein the allosteric switch linker comprises any one of the sequences of SEQ ID NO:99 to SEQ ID NO:287.

20. The bispecific molecule according to claim 1, which further comprises a third binding domain, wherein the third binding domain specifically binds
a) an antigen conferring extended half-life to the bispecific molecule, such as human serum albumin or FcRn; or
b) a cell surface antigen expressed on the surface of a cancer cell, an immune cell, a diseased cell, an infected cell, or a pathogen.

21. (canceled)

22. The bispecific molecule according to claim 1, wherein the calmodulin binding ligand is selected from a member listed in Table 4 or a polypeptide comprising a sequence selected from SEQ ID NO: 1 to SEQ ID NO:85.

23. (canceled)

24. A pharmaceutical composition comprising the bispecific molecule according to claim 1, a calmodulin binding ligand and a pharmaceutically acceptable carrier.

25. An isolated nucleic acid or nucleic acids encoding the bispecific molecule of claim 1.

26. A vector or vectors comprising the nucleic acid or nucleic acids of claim 25.

27. (canceled)

28. (canceled)

29. (canceled)

30. (canceled)

31. A method of treating cancer comprising administering an effective amount of the bispecific molecule according to claim 1 to an individual in need thereof.

32-39. (canceled)
